(19) United States
(12) Patent Application Publication
(10) Pub. No.: US 2010/0104518 A1
(43) Pub. Date: Apr. 29, 2010

(54) CHEWING GUM, CONFECTION, AND OTHER ORAL DELIVERY VEHICLES CONTAINING A TRADITIONAL CHINESE MEDICINE OR EXTRACT THEREOF

(76) Inventors: Jianwei J. Cai, Elmhurst, IL (US); Minmin Tian, Naperville, IL (US); Michael J. Greenberg, Northbrook, IL (US); Scott W. Marske, LaGrange, IL (US); Biao Che, Guangzhou (CN); Albert H. Chapdelaine, Naperville, IL (US); Zheng Xia Han, Guangzhou (CN); Wen Xiong Huang, Guangzhou (CN)

Correspondence Address: WRIGLEY & DREYFUS 28455;BRINKS HOFER GILSON & LIONE, P.O. BOX 10395, CHICAGO, IL, 60610, US

(21) Appl. No.: 12/444,680

(22) PCT Filed: Jan. 16, 2007
(86) PCT No.: PCT/US2007/060583

§ 371 Date: May 28, 2009

(30) Foreign Application Priority Data
Oct. 11, 2006 (US) PCT US2006039687

(51) Int. Cl. A61K 9/68

(52) U.S. Cl. 424/48

(57) ABSTRACT

A method for producing a chewing gum with a controlled release traditional Chinese medicine active agent, as well as the chewing gum so produced, is obtained by physically modifying the release properties of the traditional Chinese medicine active agent by coating and drying. The traditional Chinese medicine active agent is coated by encapsulation, partially coated by agglomeration, entrapped by absorption, or treated by multiple steps of encapsulation, agglomeration, and absorption. The coated traditional Chinese medicine active agent is preferably then co-dried and particle sized to produce a release-modified traditional Chinese medicine active agent for use in chewing gum. The traditional Chinese medicine active agent may also be used in a coating on a chewing gum product, as part of a rolling compound applied to the chewing gum product, or as a part of the liquid in a liquid-center chewing gum product.

### REFERENCE TO RELATED APPLICATION

The present application claims the benefit of the filing date of PCT Application Serial No. PCT/US2006/039687, filed Oct. 11, 2006, designating the United States, which is hereby incorporated by reference.

### BACKGROUND OF THE INVENTION

The present invention relates to methods for producing chewing gum, confection, and other oral delivery vehicles. More particularly, the invention relates to producing chewing gum, other confections, or other oral delivery vehicles such as sprays, foams, pearls, drops, and films, containing an effective amount of traditional Chinese medicine (“TMC”) or a hydrophilic or hydrophobic solvent or supercritical fluid extract of traditional Chinese medicines. Preferably, the active medicament that is added to the chewing gum has been treated to control its rate of release from chewing gum, or the chewing gum formulation has been modified to control the release of medicament for maximum effectiveness. Similarly, the active medicament is formulated in a confection such as a compressed mint, chewy candy, or lozenge or other oral delivery vehicle to maximize effectiveness and good taste.

In recent years, efforts have been devoted to controlling release characteristics of various ingredients in chewing gum. Most notably, attempts have been made to delay the release of sweeteners and flavors in various chewing gum formulations to thereby lengthen the satisfactory chewing time of the gum. Delaying the release of sweeteners and flavors can also avoid an undesirable overpowering burst of sweetness or flavor during the initial chewing period. On the other hand, some ingredients have been treated so as to increase their rate of release in chewing gum.

Besides sweeteners, other ingredients may require a controlled release from chewing gum. In certain embodiments, it is contemplated that the active medicament that is added to the gum is not generally released very readily. An active medicament may be encapsulated in a water soluble matrix such that, during the chewing period, the medicament may be released quickly, resulting in a fast release. This would allow chewing gum to be a carrier for an active medicament with these fast release characteristics.

In some instances, serious taste problems may arise because of the bitter nature of many active medicaments. A prolonged or delayed release of active medicaments would allow for the use of the active medicaments in gum, but the low level of release of such medicaments may keep the level of that agent below the taste threshold of the active medicaments and not give chewing gum a bitter taste quality. In addition, active medicaments may also have other unpleasant tastes that may be overcome by reducing the release rate of active medicaments from a chewing gum.

Another aspect of the present invention contemplates the use of encapsulation techniques. For example, it may be that active medicaments may also be unstable in a chewing gum environment. In such cases, various methods of encapsulation may be needed to improve stability of the active medicament. In other circumstances, active medicaments may not be readily released from the chewing gum matrix and their effect may be considerably reduced. In such a situation, a fast release encapsulation may be needed to release active medicament from the gum matrix.

Other methods contemplated are methods of controlling release of active medicament from gum. These methods would be useful in not releasing the active medicament in the oral cavity during gum chewing, but allowing the active medicament to be ingested during chewing. This will keep the active medicament from becoming effective until after it enters the digestive track.

It is of course known to provide active medicaments to individuals for various purposes. These medicaments can be used to treat diseases and as such are typically referred to as drugs or medicaments. Likewise, the drugs or medicaments can be used for preventive purposes. Still, it is known to provide medicaments to an individual for a variety of non-medical purposes including enhancing performance, improving oral care, freshening breath or maintaining general health.

There are a great variety of traditional Chinese medicines and their extracts. These medicaments run the gamut from stimulants, digestion aids, throat soothing, beauty enhancement, oral care, breath freshening, and even anti-aging. Some such medicaments are taken on an “as-needed” basis while other medicaments must be taken at regular intervals by the individual.

Typically, traditional Chinese medicines are administered in food, drugs or as medicaments administered topically, parenterally or enterally. Of course, parenteral administration is the administration of the drug intravenously directly into the blood stream. Enteral refers to the administration of the drug into the gastrointestinal tract. In either case, the goal of the drug administration is to move the drug from the site of administration towards the systemic circulation.

Oral administration of drugs such as traditional Chinese medicine and/or their extracts is by far the most common method of moving drugs towards systemic circulation. The majority of traditional Chinese medicines are formulated in food, beverages such as teas, powders, tablets or pills. Addition to foods, beverages, or teas during cooking can cause degradation of through heat, reactions with other food components, or degradation due to improper pH or water level (hydrolysis) upon storage. Formulation into chewing gum and confections can eliminate or minimize most if not all of these problems due to controlled process temperatures, encapsulation technology, and formulations with low water or adjusted pH.

When administered orally, drug absorption usually occurs due to the transport of cells across the membranes of the epithelial cells within the gastrointestinal tract. Absorption after oral administration is confounded by numerous factors. These factors include differences down the alimentary cannel in: the luminal pH; surface area per luminal volume; perfusion of tissue, bile, and mucus flow; and the epithelial membranes. See Merck Manual at page 2599.

A further issue affecting the absorption or orally administered drugs is the form of the drug. Most orally administered drugs are in the form of tablets or capsules. This is primarily for convenience, economy, stability, and patient acceptance. Accordingly, these capsules or tablets must be disintegrated or dissolved before absorption can occur. There are a variety of factors capable of varying or retarding disintegration of solid dosage forms. Further, there are a variety of factors that affect the dissolution rate and therefore determine the availability of the drug for absorption. See Merck Manual at page 2600. A factor for effecting absorption and speed of absorption is the administration of a drug with food. It has been determined by Chow et. al. that green tea catechins are poorly absorbed upon consumption with a meal vs. an empty stomach. It is believed that the catechins bind with food proteins in the stomach which greatly reduce their bioavailability.

When a drug rapidly dissolves from a drug product and readily passes across membranes, absorption from most site administration tends to be complete. This is not always the case for drugs given orally. Before reaching the vena cava, the drug must move down the alimentary canal and pass through the gut wall and liver, which are common sites of drug metabolism. Thus, the drug may be metabolized before it can be measured in the general circulation. This cause of a decrease in drug input is called the first pass effect. A large number of drugs show low bioavailability owning to an extensive first pass metabolism. The two other most frequent causes of low bioavailability are insufficient time in the GI tract and the presence of competing reactions. See Merck Manual at page 2602.

Bioavailability considerations are most often encountered for orally administered drugs. Differences in bioavailability can have profound clinical significance.

Although parenteral administration does provide a method for eliminating a number of the variables that are present with oral administration, parenteral administration is not a preferable route. Typically parenteral administration requires the use of medical personnel and is just not warranted nor practical for the administration of most agents and drugs, e.g., analgesics. Even when required, parenteral administration is not preferred due to patient concerns including comfort, infection, etc., as well as the equipment and costs involved.

There is therefore a need for an improved method of delivering drugs and agents to an individual.

### SUMMARY OF THE INVENTION

The present invention provides improved methods for delivering a traditional Chinese medicine or its extract to an individual. To this end, chewing gum is provided including a medicament or active agent. The medicament or active agent is present within the chewing gum composition (the water soluble portion and/or insoluble base portion). It has been found that by chewing the gum, the medicament or active agent is released from the chewing gum into saliva. Possibly, saliva coats the oral tissues under the tongue (sublingual) and the sides of the mouth where the drug may partition from the saliva into the oral mucosa. Continuing to chew the chewing gum creates a pressure within the buccal cavity and may force the active agent or medicament directly into the systemic system of the individual through the oral mucosa contained in the buccal cavity. This greatly enhances the absorption of the drug into the systemic system, as well as the bioavailability of the drug within the system.

Improved chewing gum formulations including medicaments and active agents are also provided by the present invention.

To this end, the present invention provides a method of active delivery comprising the steps of: providing a chewing gum that includes a medicament in the chewing gum composition; chewing the chewing gum to cause the medicament to be released from the chewing gum composition into the buccal cavity of the chewer.

The present invention also provides active delivery comprising the steps of providing a confection that includes the active n the confection composition, sucking or chewing the confection to cause the medicament to be released into the buccal cavity of the consumer. In addition, the present invention also provides active delivery comprising the steps of providing a spray, foam, pearls, or films that includes the active medicament in the formulation of these products and spraying, chewing or sucking the product releases the active into the [symbol] oral cavity of the consumer. Absorption can take place sublingually, bucally or through the gastrointestinal system.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The active medicament used in the present invention may be any agent that is traditionally used as a health aid, or medicament and lends itself to being administered through the oral cavity. The term “traditional Chinese medicine active agent” as used herein and in the claims includes traditional Chinese medicines, or hydrophilic or hydrophobic solvent extracts thereof. Such active agents may be:

Fructus Amomi seed ( [symbol] Sa-ren)

### Fructus Citri Sarcodactylis (Fu-shou)

### Ginger (Shen-jian)

Cardamom ( [symbol] Bai-dou kou)
Iron/black sesame ( [symbol] Hei-zi-ma)
Blueberry, or raspberry, or pomegranate extract
Bloat fruit ( [symbol] Pang-da-hai)

### Fructus Phyllanthi Emblicae (Yu-ganizi)

Arctium lappa L. ( [symbol] Niu-bang)

### Dandyline (Pu-gong-yin)

Chinese white olive and salt ( [symbol] Qing-guo)
Vitamin C and Green Tea extract

### Probiotics

Common Thistle, field thistle ( [symbol] Xiao-ji)
Indian madder root ( [symbol] Xi-cao)

### Clove (Ding-xiang)

Catechu/Sweet Osmanthus flower ( [symbol] Er-cha/Gui-hua)

### Herba Agastaches Rugosae (Huo-xiang)

### Anise (Ba-jiao-hui)

White Mulberry root-bark ( [symbol] Shang-bai-pi)

### Astragalus (Huang-qi)

### American Ginseng (Xi-yang-shen)

Spine date seed (Ziziphus jujube) ( [symbol] Suan-zao-ren)
Poria Cum radix Pini (Indian Bread with pine root) ( [symbol] Fu-shen)
Rose flower ( [symbol] Mei-gui-hua)

### Flos Citri Daidai (Dai-dai-hua)

### Prunus Mume (Bai-mei-hua)

### Aloes (Lu-hui)

### Barbary Woltberry Fruit

### Chrysanthemum Flower (Ju-hua)

### Fritillariae Bulb (Bei-mu)

### Honeysuckle Flower (Jing-yin-hua)

### Longan (Long-yan)

### Loquat Leaf (Pi-pa-ye)

Siraitia grosvenori ( [symbol] Luo-han-guo)
Aloes: 1, Aloe vera L.; 2. Aloe ferox Mill.
Barbary Wolfberry Fruit: Lycium barbarum L.
Bloat—fruited Sterculia Seed:

### Semen Sterculiae Lychnopherae

### Chinese White Olive:

### Canarium Album

### Chrysanthemum Flower

Chrysanthemum morifolium Ramat.

### Fritillariae Bulb

### Bulbus Fritillariae Unibracteatae

### Honeysuckle Flower Fols Lonicerae

Longan: Euphoria longana (Lour.)

### Loquat Leaf Folium Eriobotryae

### Luo Han Guo: Momordica Grosvenori

### Shinyleaf Pricklyash (Lian-mian-zhen)

Chrysanthemum morifolium ( [symbol] Jin-ju-hua)
Eapatorium japonicum ( [symbol] Pei-lan)
Coptis chinensis ( [symbol] Chuan-lian)
Paris polyphylla ( [symbol] Zao-xiu)

### Herba Patriniae (Bai-jiang-cao)

Artenzisia capillaris ( [symbol] Yin-chen)
Viola yedoensis ( [symbol] Zi-hua-di-ding)
Oidenlandia diffusa ( [symbol] She-she-cao)
Acanthopanax gracilistylus ( [symbol] Wu-jia-pi)
Agriunzonia pilosa ( [symbol] Xian-he-cao)
Ampelopsis japonica ( [symbol] Bai-lian)
Angelica dahurica ( [symbol] EBai-zhi)
Baphicacanthus cusia ( [symbol] Qin-dai)
Carthamus tinclorius ( [symbol] Hong-hua)
Chrysanthemum moriflolium ( [symbol] Bai-ju-hua)
Cnidium monnieri ( [symbol] She-chuang-zi)
Dryobalanops aromatica ( [symbol] Bing-pian)
Euphorbia pekinesis ( [symbol] Da-ji)
Houttuymia cordata ( [symbol] Yu-xin-cao)
Isatis tinctoria ( [symbol] Ban-lan-geng)
Isodon striatus ( [symbol] Xi-huang-cao)
Jasminum nudiflorum ( [symbol] Ying-chun-hua)
Juncus effuses ( [symbol] Deng-xin-hua)
Lasiosphaera fenzlii ( [symbol] Ma-bo)
Ligusticum chuanxiong ( [symbol] Chuan-xiong)
Lithospermum erythrorhizon ( [symbol] Zi-cao)
Lonicera japonica ( [symbol] Jin-ying-hua)
Morus alba ( [symbol] Sang-bai-pi)
Nelumbo nucifera ( [symbol] Lian-zi-xin)
Ophiopogon japonicus ( [symbol] Mai-dong)
Paeonia lactiflora ( [symbol] Bai-shao)
Paeonia suffruticosa ( [symbol] Mu-dan-pi)
Phellodendron amurense ( [symbol] Huang-bai)
Polygonum cuspidatum ( [symbol] Hu-zhang)
Prunella vulgaris ( [symbol] Xia-ku-cao)
Prunus mume ( [symbol] Wu-mei-gan)
Rhus chinensis ( [symbol] Wu-bei-zi)
Sargassum fusiforme ( [symbol] Hai-zhao)
Stemon japonica ( [symbol] Bai-bu)
Sophora flavescens ( [symbol] Ku-shen)

### Clove (Ding-xian)

Taraxacum mongolicum ( [symbol] Pu-gong-ying)
Zanthoxylum bungeanum ( [symbol] Hua-jiao)
Ligusticum chuanxiong, extract ( [symbol] Chuan-xiong-qin)
Cnidium monnieri, extract ( [symbol] She-chuang-zi shu)
Alpinia japonica, extract ( [symbol] Shang-jian-su)
Rheum palmatum extract ( [symbol] Da-huang-su)
Ginko extract ( [symbol] Ying-xin Extract)
Puerarin extract ( [symbol] Ge-geng-su)
Forsythia suspensa, extract ( [symbol] Lian-qiao-gan)
Scutellaria baicalensis ( [symbol] Huang-qi-gan)

Accordingly, an advantage of the present invention is to provide new methods for delivering medicaments or active agents to an individual.

Still further, an advantage of the present invention is to provide a method of delivering medicaments to an individual that provides for improved stability of active medicaments allowing a known dose to be delivered orally, and increase absorption and bioavailability as compared to medicaments that are formulated in foods and beverages and are designed to be absorbed in the GI tract.

Further, an advantage of the present invention is to provide a method of administering a medicament or agent to an individual at a lower level than is typically administered orally while still achieving the same effect.

Furthermore, an advantage of the present invention is to provide a method for administering medicament actives to an individual that heretofore were administered parenterally.

Additionally, an advantage of the present invention is to provide a method of administering drugs that is more palatable than current methods.

Moreover, an advantage of the present invention is to provide an improved delivery method for traditional Chinese medicine or there extracts.

The present invention also provides a method of producing chewing gum with physically modified active medicaments to control their release. Such active medicaments are added to a gum coating to deliver the active medicaments systemically without unpleasant tastes. The present invention also relates to the chewing gum so produced. Physically modified active medicaments may be added to sucrose-type gum formulations and sucrose-type coatings. The formulation may be a low or high moisture formulation containing low or high amounts of moisture containing syrup. Physically modified active medicaments may also be used in low or non-sugar gum formulations and coatings that use sorbitol, mannitol, other polyols or carbohydrates. Non-sugar formulations may include low or high moisture sugar-free chewing gums.

Active medicaments described herein may be combined or co-dried with bulk sweeteners typically used in chewing gum before the active medicaments are physically modified. Such bulk sweeteners are sucrose, dextrose, fructose and maltodextrins, as well as sugar alcohols such as sorbitol, mannitol, xylitol, maltitol, lactitol, hydrogenated isomaltulose and hydrogenated starch hydrolyzates.

The modified release rate noted above may be a fast release or a delayed release. The modified release of active medicaments may be obtained by encapsulation, partial encapsulation or partial coating, entrapment or absorption with high or low water soluble materials or water insoluble materials. The procedures for modifying the active medicaments include spray drying, spray chilling, fluid bed coating, coacervation, extrusion and other agglomerating and standard encapsulating techniques. The active medicaments also may be absorbed onto an inert or water-insoluble material. Active medicaments may be modified in a multiple step process comprising any of the processes, or a combination of the processes noted. Prior to encapsulation, active medicaments may also be combined with bulk sweeteners including sucrose, dextrose, fructose, maltodextrin or other bulk sweeteners, as well as sugar alcohols such as sorbitol, mannitol, xylitol, maltitol, lactitol, hydrogenated isomaltulose and hydrogenated starch hydrolyzates.

Prior to encapsulation, active medicaments may be combined with high-intensity sweeteners, including but not limited to thaumatin, aspartame, alitame, acesulfame K, saccharin acid and its salts, glycyrrhizin, cyclamate and its salts, stevioside and dihydrochalcones. Co-encapsulation of active medicaments along with a high-intensity sweetener may reduce the poor taste qualities of active medicaments and control the sweetener release with active medicaments. This can improve the quality of the gum product and increase consumer acceptability.

The preparation of confectionery formulations is historically well known and has changed little through the years. Confectionery items have been classified as either “hard” confectionery or “soft” confectionery. The traditional Chinese medicines of the present invention may be incorporated into confectionery compositions by admixing the inventive composition into conventional hard and soft confections.

As used herein, the term confectionery material means a product containing a bulking agent selected from a wide variety of materials such as sugar, corn syrup, and in the case of sugarless bulking agents, sugar alcohols such as sorbitol, xylitol and mannitol and mixtures thereof. Confectionery material may include such exemplary substances as lozenges, tablets, toffee, nougat, suspensions, chewy candy, chewing gum and the like. The bulking agent is present in a quantity sufficient to bring the total amount of composition to 100%. In general, the bulking agent will be present in amounts up to about 99.98%, preferably in amounts up to about 99.9%, and more preferably in amounts up to about 99%, by weight of the traditional Chinese medicines composition.

Lozenges are flavored dosage forms intended to be sucked and held in the mouth. Lozenges may be in the form of various shapes such as flat, circular, octagonal and biconvex forms. The lozenge bases are generally in two forms: hard boiled candy lozenges and compressed tablet lozenges.

Hard boiled candy lozenges may be processed and formulated by conventional means. In general, a hard boiled candy lozenge has a base composed of a mixture of sugar and other carbohydrate bulking agents kept in an amorphous or glassy condition. This amorphous or glassy form is considered a solid syrup of sugars generally having from about 0.5% to about 1.5% moisture. Such materials normally contain up to about 92% corn syrup, up to about 55% sugar and from about 0.1% to about 5% water, by weight of the final composition. The syrup component is generally prepared from corn syrups high in fructose, but may include other materials. Further ingredients such as flavoring agents, sweetening agents, acidulants, coloring agents and the like may also be added.

Boiled candy lozenges may also be prepared from non-fermentable sugars such as sorbitol, mannitol, and hydrogenated corn syrup. Typical hydrogenated corn syrups are Lycasin, a commercially available product manufactured by Roquette Corporation, and Hystar, a commercially available product manufactured by Lonza, Inc. The candy lozenges may contain up to about 95% sorbitol, a mixture of sorbitol and mannitol in a ratio from about 9.5:0.5 up to about 7.5:2.5, and hydrogenated corn syrup up to about 55%, by weight of the solid syrup component.

Boiled candy lozenges may be routinely prepared by conventional methods such as those involving fire cookers, vacuum cookers, and scraped-surface cookers also referred to as high speed atmospheric cookers.

Fire cookers involve the traditional method of making a boiled candy lozenge base. In this method, the desired quantity of carbohydrate bulking agent is dissolved in water by heating the agent in a kettle until the bulking agent dissolves. Additional bulking agent may then be added and cooking continued until a final temperature of 145° C. to 156° C. is achieved. The batch is then cooled and worked as a plastic-like mass to incorporate additives such as flavors, colorants and the like.

A high-speed atmospheric cooker uses a heat-exchanger surface which involves spreading a film of candy on a heat exchange surface, the candy is heated to 165° C. to 170° C. in a few minutes. The candy is then rapidly cooled to 100° C. to 120° C. and worked as a plastic-like mass enabling incorporation of the additives, such as flavors, colorants and the like.

In vacuum cookers, the carbohydrate bulking agent is boiled to 125° C. to 132° C., vacuum is applied and additional water is boiled off without extra heating. When cooking is complete, the mass is a semi-solid and has a plastic-like consistency. At this point, flavors, colorants, and other additives are admixed in the mass by routine mechanical mixing operations.

The optimum mixing required to uniformly mix the flavoring agents, coloring agents and other additives during conventional manufacturing of boiled candy lozenges is determined by the time needed to obtain a uniform distribution of the materials. Normally, mixing times of from 4 to 10 minutes have been found to be acceptable.

Once the boiled candy lozenge has been properly tempered, it may be cut into workable portions or formed into desired shapes. A variety of forming techniques may be utilized depending upon the shape and size of the final product desired. A general discussion of the composition and preparation of hard confections may be found in H. A. Lieberman, Pharmaceutical Dosage Forms: Tablets, Volume I (1980), Marcel Dekker, Inc., New York, N.Y. at pages 339 to 469, which disclosure is incorporated herein by reference.

The apparatus useful in accordance with the present invention comprises cooking and mixing apparatus well known in the confectionery manufacturing arts, and therefore the selection of the specific apparatus will be apparent to the artisan.

In contrast, compressed tablet confections contain particulate materials and are formed into structures under pressure. These confections generally contain sugars in amounts up to about 95%, by weight of the composition, and typical tablet excipients such as binders and lubricants as well as flavoring agents, coloring agents and the like. The pressed tablet into which the traditional Chinese medicines are incorporated may be prepared by wet granulation, dry granulation, and direct compression methods. These methods involve conventional procedures well known to the ordinary skilled artisan. In general, wet granulation involves mixing milled powders, preparing a wet mass by blending the milled powders with a binder solution, coarse screening the wet mass and drying the moist granules, screening the granules through a 14 to 20 mesh screen, mixing the screened granules with lubricants and disintegrate agents and finally tablet compressing the mass. In contrast, dry granulation generally involves milling of powders, compression into large hard tablets to make slugs, screening of slugs, mixing with lubricants and disintegrating agents and finally tablet compression. In the direct compression method, the milled ingredients are mixed and then merely tableted by compression.

In addition to hard confectionery materials, the lozenges of the present invention may be made of soft confectionery materials such as those contained in nougat. The preparation of soft confections, such as nougat, involves conventional methods, such as the combination of two primary components, namely (1) a high boiling syrup such as a corn syrup, hydrogenated starch hydrolysate or the like, and (2) a relatively light textured frappe, generally prepared from egg albumin, gelatin, vegetable proteins, such as soy derived compounds, sugarless milk derived compounds such as milk proteins, and mixtures thereof. The frappe is generally relatively light, and may, for example, range in density from about 0.5 to about 0.7 grams/cc.

The high boiling syrup, or “bob syrup” of the soft confectionery is relatively viscous and has a higher density than the frappe component, and frequently contains a substantial amount of carbohydrate bulking agent such as a hydrogenated starch hydrolysate. Conventionally, the final nougat composition is prepared by the addition of the “bob syrup” to the frappe under agitation, to form the basic nougat mixture. Further ingredients such as flavoring agents, additional carbohydrate bulking agent, coloring agents, preservatives, medicaments, mixtures thereof and the like may be added thereafter also under agitation. A general discussion of the composition and preparation of nougat confections may be found in B. W. Minifie, Chocolate, Cocoa and Confectionery: Science and Technology, 2nd edition, AVI Publishing Co., Inc., Westport, Conn. (1980), at pages 424-425, which disclosure is incorporated herein by reference.

The procedure for preparing the soft confectionery involves known procedures. In general, the frappe component is prepared first and thereafter the syrup component is slowly added under agitation at a temperature of at least about 65° C., and preferably at least about 100° C. The mixture of components is continued to be mixed to form a uniform mixture, after which the mixture is cooled to a temperature below 80° C., at which point, the flavoring agent may be added. The mixture is further mixed for an additional period until it is ready to be removed and formed into suitable confectionery shapes.

Chewable traditional Chinese medicine candy is prepared by procedures similar to those used to make soft confectionery. In a typical procedure, a boiled sugar-corn syrup blend is formed to which is added a frappe mixture. The boiled sugar-corn syrup blend may be prepared from sugar and corn syrup blended in parts by weight ratio of about 90:10 to about 10:90. The sugar-corn syrup blend is heated to temperatures above about 120° C. to remove water and to form a molten mass. The frappe is generally prepared from gelatin, egg albumin, milk proteins such as casein, and vegetable proteins such as soy protein, and the like, which is added to a gelatin solution and rapidly mixed at ambient temperature to form an aerated sponge like mass. The frappe is then added to the molten candy mass and mixed until homogeneous at temperatures between about 65° C. and about 120° C.

The ingestible traditional Chinese medicine composition of the invention can then be added to the homogeneous mixture as the temperature is lowered to about 65° C.-95° C. whereupon additional ingredients can then be added such as flavoring agents and coloring agents. The formulation is further cooled and formed into pieces of desired dimensions.

The present invention provides improved methods for delivering traditional Chinese medicines or their hydrophilic or hydrophobic solvent extracts, medicaments and other active agents to an individual, as well as improved formulations including such medicaments and agents. Pursuant to the present invention, a physically modified medicament or active is contained in a chewing gum formulation. In contrast to some prior such formulations, the medicament or agent is contained directly in the chewing gum composition.

Accordingly, as the chewing gum is chewed, the physically modified active is released into the saliva. During continual chewing, the medicament or active in the saliva may be then forced due to the pressure created by the chewing gum through the oral mucosa in the buccal cavity. The oral mucosa favors drug absorption. In contrast to a typically oral ingested drug, wherein the solution is in contact too briefly for absorption to be appreciable through the oral mucosa, it is believed that during the chewing, the physically modified active agent and/or medicament remains in the buccal cavity and may be forced or partitioned through the oral mucosa. An increase in the absorption of the drug may be achieved as well as an increase in the bioavailability of the drug as compared to typical oral administration. The drug or active agent may be absorbed much quicker than if it was swallowed as in a typical oral administration. Indeed, the absorption approaches that of a parental administration and bioavailability may be also much greater than oral administration.

It is also possible that less physically modified medicament or active agent can be placed in the chewing gum than is typically orally administered to an individual to achieve an effect and the same bioequivalence can be achieved. In some instances, for certain drugs and agents, the administration of the medicament or agent using chewing gum through the buccal activity may provide an increase in therapeutic effect even as compared to parenteral administration.

For example, caffeine is commonly used as a stimulant to alleviate the effects of sleep deprivation. It is almost completely metabolized in the liver and therefore classified as a low clearance, flow independent drug. This means its rate of inactivation is unaffected by delivery to the liver and can only be modified by a change in the hepatic enzyme activity.

Data set forth in detail in U.S. patent application Ser. No. 09/386,818 herein incorporated by reference, suggests that the absorption rate constant (Ka) is significantly increased when caffeine is administrated through chewing gum versus a pill. This means that the caffeine is moving into the systemic circulation at a significantly faster rate. A similar change in the onset of dynamic response has also been noted, e.g., alertness and performance.

When caffeine is added to stick chewing gum at a level of about 0.2% to about 5%, caffeine imparts an intense bitterness to the chewing gum that lasts throughout the chewing period. The higher the level used, the stronger the bitterness. At about 0.2%, which is about 5 mg per 2.7 gram stick, the bitterness is below the threshold limit and is not readily discernible. Taste limits in stick chewing gum are generally about 0.4% (10 mg) to about 4% (100 mg) of caffeine in a stick of gum. The 60-80 mg level of caffeine is about the level of caffeine found in a conventional cup of coffee. The target level of caffeine in stick gum is about 40 mg per stick, with a range of about 25-60 mg, so that a five stick package of gum would contain about 200 mg of caffeine, or the equivalent of caffeine in two strong cups of coffee. However, at this level caffeine bitterness overwhelms the flavor initially and lasts throughout the chewing period.

For coated pellet gum, piece weight is generally about 1.5 grams per piece. However, one coated piece of gum is about equal to ½ piece of stick gum. Two pellets are equivalent to a stick of gum, and together weigh about 3 grams. The above-noted target level of 40 mg per stick is equivalent to 20 mg per coated piece, or a range of about 12 to 30 mg caffeine per piece. This is about 0.8% to about 2% caffeine in a piece of coated gum, or a target level of 1.3%.

Caffeine is a slightly water soluble substance and, therefore, has a moderately slow release from stick chewing gum. Caffeine is 2.1% soluble in water at room temperature, 15% soluble in water at 80° C. and 40% soluble in boiling water. This gives caffeine a moderately slow release as shown below:

| | |
|---|---|
| Chewing Time | % Caffeine Release |

| | | |
|---|---|---|
| 0 | min | — |
| 5 | min | 56 |
| 10 | min | 73 |
| 20 | min | 88 |
| 40 | min | 97 |

Generally, highly water soluble ingredients such as sugars in stick gum are about 80-90% released after only five minutes of chewing. For caffeine, only about 50% is released, while the other 50% remains in the gum after five minutes of chewing. After 20 minutes almost 90% of caffeine is released.

Even if caffeine is dissolved in hot water and mixed in the stick gum, when the gum is cooled or kept at room temperature, caffeine may return to its normal crystalline state and release at a rate similar to that shown above.

When a physically modified active such as caffeine is added to a gum stick, the active agent will have an increased water solubility, and release quickly into the mouth from the gum. Depending on the active agent, which may generally be non water soluble, physically modifying the active agent by various forms at encapsulation will increase the release of the active agent from chewing gum. Most water soluble active agents can be modified by encapsulation to give a more uniform release from chewing gum. Depending on the active agent and the type of encapsulation used, the level released from the gum into the mouth can be adjusted for maximum effectiveness.

Other agents or medicaments may be included in the present invention. The traditional Chinese medicine or its hydrophilic or hydrophobic solvent extract will normally have a desired therapeutic or physiological effect once ingested and/or metabolized. The therapeutic effect may be one which decreases the growth of a xenobiotic or other gut flora or fauna, alters the activity of an enzyme, provides the physical relief from a malady (e.g., diminishes pain, acid reflux or other discomfort), soothes the throat, reduces internal heat, prevents oxication of LDL leading to arterial plaque build up, antibacterial activity in the oral cavity, or has an effect on the brain chemistry of molecules that determine mood and behavior. Of course these are just examples of what is intended by therapeutic effect. Those of skill in the art will readily recognize that a particular agent has or is associated with a given therapeutic effect.

The active agent may be any agent that is traditionally used as a medicament and lends itself to being administered through the oral cavity, such as:

| | |
|---|---|
| [symbol] Fructus Amomi seed | [symbol] |
| | Good for stomach, remove accumulated food, increase |
| | appetite |
| [symbol] Fructus Citri Sarcodactylis (also can | [symbol] |
| be used for soothing throat) | Good for stomach, cleanse internal circulation, remove |
| | indigestion discomfort |
| [symbol] Ginger | [symbol] |
| | Good for stomach, help digestion, stop vomiting |
| [symbol] Cardamom (also in breath | [symbol] |
| freshening) | Good for stomach, remove accumulated food, improve |
| | digestion function |
| Iron/black sesame | [symbol] |
| | Blood replenishment, improve blood nutrition |
| Blueberry, or raspberry, or pomegranate | Natural food high in antioxidants |
| extract | |
| [symbol] Chinese white olive | [symbol] |
| | Cooling and soothing throat |
| [symbol] Bloat fruit | [symbol] |
| | Soothe throat and reduce inflammation |
| [symbol] Fructus Phyllanthi Emblicae (also | [symbol] |
| can be used as digestion aid) | Soothe throat, reduce internal heat, increase saliva, stop |
| | coughing, |
| [symbol] Arctium lappa L. | [symbol] |
| | Good for lung, soothe throat, cooling internal heat, stop |
| | coughing |
| [symbol] Dandyline | [symbol] |
| | Reduce internal heat, detoxification |
| [symbol] Chinese white olive + salt | [symbol] |
| | Reduce internal heat, detoxification, reduce inflammation |
| Vitamin C + Green Tea extract | Reduce inflammation |
| Probiotics | Good bacteria kill bad bacteria in mouth |
| [symbol] Common Thistle, field thistle | [symbol] |
| | Cool blood, stop bleeding, suitable for fresh wound |
| [symbol] Indian madder root | [symbol] [symbol] |
| | In crease blood circulation, stop bleeding, cleanse blood |
| | clotting, suitable for chronic gum bleeding and bruises |
| [symbol] Clove | [symbol] |
| | Antibacterial, pungent, mild |
| [symbol] Cardamom | [symbol] |
| | Good for spleen, remove bad odor, aromatic, cleanse |
| | internal odor |
| [symbol] Catechu/Sweet Osmanthus | [symbol] |
| flower | Reduce internal heat, remove bad odor, aromatic, cleanse |
| | internal odor |
| [symbol] Herba Agastaches Rugosae | [symbol] |
| | Aromatic, reduce heat stroke, stop vomiting |
| [symbol] Anise (can be used in | Aromatic, breath freshening |
| combination with licorice) | |
| [symbol] White Mulberry root-bark | [symbol] |
| | Cleanse lung and remove sputum |
| [symbol] Huang Qi (Astragalus) | [symbol] |
| | Warm energy, strengthen body, suitable for people with |
| | physical weakness |
| [symbol] American Ginseng | [symbol] [symbol] |
| | Cool energy, replenish body and restore energy, suitable fro |
| | people with fatigue due to stress |
| [symbol] Spine date seed (Ziziphus jujube) | [symbol] |
| | Calming and relax mind |
| [symbol] Poria Cum radix Pini (Indian | [symbol] |
| Bread with pine root) | Good for spleen and calming |
| [symbol] Rose | [symbol] |
| | Reduce stress or depression and calming |
| [symbol] ( [symbol] ) Prunus Mume | [symbol] |
| | Aromatic, cleanse internal body odor, calming and reduce |
| | depression |

| Common Name | Botanical name | Pharmacological properties | TCM use and dose |
|---|---|---|---|
| Aloes | 1, Aloe vera L.; 2. Aloe | Laxative effect, wound | Cools internal heat |
| | ferox Mill. | healing, antiviral or | and moves stool; can |
| | | antibacterial effect, anti- | be used for chronic |
| | | inflammatory effects | conditions. |
| | | | Strengthens |
| | | | Stomach; used for |
| | | | digestive impairment |
| | | | in small children. |
| | | | Clears liver |
| Barbary Wolfberry Fruit | Lycium barbarum L. | Effects on treating fatty liver in | Nourish yin, enrich |
| (Dried fruits of Lycium | | animals due to betaine | blood, benefit |
| barbarum L. plants) | | | essence and improve |
| | | | visual acuity: For |
| | | | deficiency of liver- |
| | | | yin and kidney-yin |
| | | | and insufficiency of |
| | | | essence and blood |
| | | | manifested as |
| | | | dizziness, blurring of |
| | | | vision, hypopsia, |
| | | | tinnitus, emission |
| | | | and soreness of the |
| | | | loin and extremities; |
| | | | also for diabetes. |
| Bloat-fruited Sterculia | Semen Sterculiae | Laxative effect in animals | Relieve coughing, |
| Seed | Lychnopherae | Blood pressure reduction effect | cool internal heat; |
| (Dried seeds of a Sterculia | | in animals | Transform hot |
| Scaphigera tree) | | | phlegm; Clears colon |
| | | | and moves stool. |
| | | | Expels phlegm and |
| | | | clears lung heat. |
| | | | Used for sore throat, |
| | | | chronic throat |
| | | | inflammation, and |
| | | | constipation. Use 1.5- |
| | | | 3 g. |
| Chinese White Olive | Canarium Album | Effects on treating acute | Clears lung, moistens |
| (Dried Chinese olive) | | diarrhea, skin infection | throat, clears throat, |
| | | | generates saliva, |
| | | | reduces internal |
| | | | poison |
| Chrysanthemum Flower | Chrysanthemum | Anti-bacteria effects | Relieve internal heat, |
| (dried flower) | morifolium Ramat. | Anti-inflammatory effects | Regulate Qi; |
| | | | Extinguish wind heat |
| | | | with fever and |
| | | | headache. Clears |
| | | | liver and brightens |
| | | | eyes. Pacifies liver |
| | | | and extinguishes |
| | | | internal wind with |
| | | | dizziness headache. |
| | | | Dose 4.5-9 g |
| Fritillariae Bulb | Bulbus Fritillariae | Fritimine: has sustained blood | Cooling, reduce |
| (Dried bulb of a Lily | Unibracteatae | pressure reduction effect in | internal heat, moisten |
| family flower) | | animals | lung, reduce phlegm, |
| | | Sipeimine: has sustained blood | stop coughing, |
| | | pressure reduction effect in | normally use 2-3 g. |
| | | animals | |
| Honeysuckle Flower | Fols Lonicerae | Anti-bacteria effects | Clears internal heat |
| (Dried flower) | | | and detoxifies Fire |
| | | | Poison; especially of |
| | | | breast, throat, eyes, |
| | | | or intestines. Dose is |
| | | | 6-15 g. used for sore |
| | | | throat, pneumonia, |
| | | | external infection, |
| | | | eye infection |
| Longan | Euphoria longana | Has in vitro anti-bacteria | Tonify blood, heart |
| (dried fruit) | (Lour.) | properties | and spleen, calms |
| | | | spirit. |
| | | | Good for fatigue, |
| | | | feeling weak, sleep |
| | | | disorders. Every |
| | | | time use 3-9 g. |
| Loquat Leaf | Folium Eriobotryae | No anti-bacteria effect | Relieve coughing, |
| (Dried leaf of loquat tree, | | | cool internal heat; |
| Loquat is a fruit) | | | Transform hot |
| | | | phlegm; Expels |
| | | | phlegm and clears |
| | | | lung. Harmonizes |
| | | | stomach and clear |
| | | | stomach heat, |
| Luo Han Guo | Momordica Grosvenori | | Expels phlegm and |
| (Dried fruit of | | | clears lung heat, |
| Momordica Grosvenori | | | Clears colon and |
| plant) | | | moves stool. Good |
| | | | for coughing, sore |
| | | | throat, and |
| | | | constipation |

| No. | English/Botanic Name | Chinese Name |
|---|---|---|
| 1 | Shinyleaf Pricklyash | [symbol] |
| 2 | Chrysanthemum morifolium | [symbol] |
| 3 | Eapatorium japonicum | [symbol] |
| 4 | Coptis chinensis | [symbol] |
| 5 | Paris polyphylla | [symbol] |
| 6 | Herba Patriniae | [symbol] |
| 7 | Artemisia capillaries | [symbol] |
| 8 | Viola yedoensis | [symbol] |
| 9 | Oidenlandia diffusa | [symbol] |
| 10 | Acanthopanax gracilistylus | [symbol] |
| 11 | Agriumonia pilosa | [symbol] |
| 12 | Ampelopsis japonica | [symbol] |
| 13 | Angelica dahurica | [symbol] |
| 14 | Baphicacanthus cusia | [symbol] |
| 15 | Carthamus tinclorius | [symbol] |
| 16 | Chrysanthemum moriflolium | [symbol] |
| 17 | Cnidium monnieri | [symbol] |
| 18 | Dryobalanops aromatica | [symbol] |
| 19 | Euphorbia pekinesis | [symbol] |
| 20 | Houttuymia cordata | [symbol] |
| 21 | Isatis tinctoria | [symbol] |
| 22 | Isodon striatus | [symbol] |
| 23 | Jasminum nudiflorum | [symbol] |
| 24 | Juncus effuses | [symbol] |
| 25 | Lasiosphaera fenzlii | [symbol] |
| 26 | Ligusticum chuanxiong | [symbol] |
| 27 | Lithospermum erythrorhizon | [symbol] |
| 28 | Lonicera japonica | [symbol] |
| 29 | Morus alba | [symbol] |
| 30 | Nelumbo nucifera | [symbol] |
| 31 | Ophiopogon japonicus | [symbol] |
| 32 | Paeonia lactiflora | [symbol] |
| 33 | Paeonia suffruticosa | [symbol] |
| 34 | Phellodendron amurense | [symbol] |
| 35 | Polygonum cuspidatum | [symbol] |
| 36 | Prunella vulgaris | [symbol] |
| 37 | Prunus mume | [symbol] |
| 38 | Rhus chinensis | [symbol] |
| 39 | Sargassum fusiforme | [symbol] |
| 40 | Stemon japonica | [symbol] |
| 41 | Sophora flavescens | [symbol] |
| 42 | Syzygium aromaticum | [symbol] |
| 43 | Taraxacum mongolicum | [symbol] |
| 44 | Zanthoxylum bungeanum | [symbol] |
| 45 | Ligusticum chuanxiong, extract | [symbol] |
| 46 | Cnidium monnieri, extract | [symbol] |
| 47 | Alpinia japonica, extract | [symbol] |
| 48 | Rheum palmatum extract | [symbol] |
| 49 | Ginko extract | [symbol] |
| 50 | Puerarin extract | [symbol] |
| 51 | Forsythia suspensa, extract | [symbol] |
| 52 | Scutellaria baicalensis | [symbol] |

### Test of TCM Against Oral Bacteria Responsible for Stale Breath

Chewing gum and compressed mint are often used by consumers as portable oral care and breath freshening products against oral malodor. Oral malodor, often referred to as morning breath or stale breath, is a major social and psychological problem that affects 50% general population. Volatile sulfur compounds (VSC) including hydrogen sulfide, methyl mercaptan and dimethyl sulfide are the principal materials that impart malodor. The malodorous volatile sulfuric compounds are generated through the metabolic activities of oral microorganisms on proteinaceous materials from food or saliva. Gram negative bacteria predominantly at the dorsum of the tongue are considered to be the most important group of microorganisms in the production of oral malodor. Treatments of oral malodor are often based on the following three principles: (1) masking malodor by flavors; (2) complex the VSC to form non-volatile substances; (3) kill the germs that cause bad breath. Among three approaches, control of salivary bacteria is considered to be the best. Many TCMs can be effectively used for controlling oral bacteria.

To evaluate the germ-kill efficacy, the dried crude TCM bark, stem, leaf or root was ground to powder. 10-50 grams of TCM powder was mixed with 100 ml of ethanol. The mixture was super-sonicated for 10-15 minutes to extract the TCM actives. The mixture was further transferred into a Mixxor solid-liquid phase extractor for extracting about 50 strokes. The solution was filtered by a 0.45 um Millipore filter. For determination of the TCM extract concentration, slight amount of TCM extract sample was accurately weighed on a pre-weighed aluminum weighing dish. It was dried in a 45° C. oven for 2 hours to evaporate ethanol. The sample dish was weighed again, and the concentration of TCM extract was determined as: conc.=(Dry weight−dish weight)/(wet weight−dish weight)×100%.

The germ-kill efficacy was determined by a Minimum Inhibitive Concentration test (MIC) and a Minimum Bactericidal Concentration (MBC) test. In the MIC test, the TCM extract was added to a Schaedler broth supplemented by 1 ppm of Vitamin K and 10 ppm Hemin. The solution was then serially diluted two-fold so that each subsequent dilution contained 50% of the compound concentration of the previous dilution while maintaining a constant level of nutrients for each dilution. These dilutions were inoculated with representative oral microorganisms, and incubated for 24 hrs at 37° C. For each TCM compound, the lowest dilution that was not turbid was registered as the MIC. The MBC was determined by transferring 10 um of liquid from no-turbid tubes to fresh growth media and incubated for 48 hrs. For each TCM extract, the lowest dilution that did not demonstrate growth was considered as the MBC. Table A & B list the MIC and MBC data on P. gingivalis, F. nucleatum and S. nutans.

TABLE A
MIC of TCMs on Oral Bacteria

| TCM extract | P. gingivalis μg/ml | F. nucleatum μg/ml | S. mutans μg/ml |
|---|---|---|---|
| Shinyleaf Pricklyash | 5 | 2.5 | 2.5 |
| Chrysanthemum morifolium | 5 | 1.25 | 5 |
| Eapatorium japonicum | 1.25 | 5 | 5 |
| Coptis chinensis (Chuang Lian) | 0.052 | 0.106 | 0.3125 |
| Paris polyphylla | 5 | 5 | 2.5 |
| Herba Patriniae | 1.25 | 2.5 | 2.5 |
| Artemisia capillaries | 2.5 | 1.25 | 5 |
| Viola yedoensis | 2.5 | 2.5 | 10 |
| Oidenlandia diffusa | 0.625 | 5 | 10 |
| Acanthopanax gracilistylus | 0.625 | 2.5 | 10 |
| Agriumonia pilosa | 1.25 | 1.25 | 5 |
| Ampelopsis japonica | 5 | 5 | 5 |
| Angelica dahurica | 5 | 5 | 5 |
| Baphicacanthus cusia | 0.3125 | 10 | 10 |
| Carthamus tinclorius | 5 | 5 | 5 |
| Chrysanthemum moriflolium | 0.625 | 1.25 | 0.625 |
| Cnidium monnieri | 0.106 | 2.5 | 1.25 |
| Dryobalanops aromatica | 10 | 10 | 10 |
| Euphorbia pekinesis | 2.5 | 5 | 5 |
| Houttuymia cordata | 2.5 | 5 | 2.5 |
| Isatis tinctoria | 5 | 10 | 5 |
| Isodon striatus | 1.25 | 0.3125 | 1.25 |
| Jasminum nudiflorum | 5 | >20 | >20 |
| Juncus effuses | 2.5 | 2.5 | 1.25 |
| Lasiosphaera fenzlii | 5 | 5 | >25 |
| Ligusticum chuanxiong | 2.5 | 5 | 5 |
| Lithospermum erythrorhizon | 0.106 | 0.3125 | 0.053 |
| Lonicera japonica | 2.5 | 2.5 | 2.5 |
| Morus alba | 0.3125 | 2.5 | 1.25 |
| Nelumbo nucifera | 2.5 | 10 | >20 |
| Ophiopogon japonicus | 2.5 | 10 | 5 |
| Paeonia lactiflora | 2.5 | 5 | 1.25 |
| Paeonia suffruticosa | 2.5 | 5 | 5 |
| Phellodendron amurense | 1.25 | 2.5 | 2.5 |
| Polygonum cuspidatum | 5 | 1.25 | 1.25 |
| Prunella vulgaris | 1.25 | 0.625 | 5 |
| Prunus mume | 0.625 | 2.5 | 5 |
| Rhus chinensis | 0.625 | 0.625 | 0.625 |
| Sargassum fusiforme | 0.625 | 2.5 | 2.5 |
| Stemon japonica | 5 | 10 | 10 |
| Sophora flavescens | 10 | 10 | 5 |
| Syzygium aromaticum | 2.5 | 0.625 | 2.5 |
| Taraxacum mongolicum | 5 | 5 | 10 |
| Zanthoxylum bungeanum | 2.5 | 5 | 5 |
| Ligusticum chuanxiong, extract | 1.25 | 0.3125 | 2.5 |
| Cnidium monnieri, extract | 5 | 5 | 5 |
| Alpinia japonica, extract | >10 | 1.25 | 2.5 |
| Rheum palmatum extract | 0.3125 | 5 | 2.5 |
| Ginko extract | 2.5 | 1.25 | 1.25 |
| Puerarin extract | >10 | 2.5 | >10 |
| Forsythia suspense, extract | >10 | >10 | >10 |
| Scutellaria baicalensis | 1.25 | 2.5 | 5 |
| Magnolia Bark Extract | 0.625 | 0.106 | 0.3125 |
| Chlorohexdine Gluconate* | 0.0025 | 0.005 | 0.005 |

*used as a positive control

TABLE B
MBC of TCMs on Oral Bacteria

| TCM Extract | P. gingivalis μg/ml | F. nucleatum μg/ml | S. mutans μg/ml |
|---|---|---|---|
| Shinyleaf Pricklyash | 10 | 5 | 5 |
| Chrysanthemum morifolium | 10 | 2.5 | 10 |
| Eapatorium japonicum | 2.5 | 10 | 10 |
| Coptis chinensis (Chuang Lian) | 0.106 | 0.3125 | 0.625 |
| Paris polyphylla | 10 | 10 | 5 |
| Herba Patriniae | 2.5 | 5 | 5 |
| Artemisia capillaries | 5 | 2.5 | 10 |
| Viola yedoensis | 5 | 5 | 20 |
| Oidenlandia diffusa | 1.25 | 10 | 20 |
| Acanthopanax gracilistylus | 1.25 | 5 | 20 |
| Agriumonia pilosa | 2.5 | 2.5 | 10 |
| Ampelopsis japonica | 10 | 10 | 10 |
| Angelica dahurica | 10 | 10 | 10 |
| Baphicacanthus cusia | 0.625 | 20 | 20 |
| Carthamus tinclorius | 10 | 10 | 10 |
| Chrysanthemum moriflolium | 1.25 | 2.5 | 1.25 |
| Cnidium monnieri | 0.3125 | 5 | 2.5 |
| Dryobalanops aromatica | 20 | 20 | 20 |
| Euphorbia pekinesis | 5 | 10 | 10 |
| Houttuymia cordata | 5 | 10 | 5 |
| Isatis tinctoria | 10 | 20 | 10 |
| Isodon striatus | 2.5 | 0.625 | 2.5 |
| Jasminum nudiflorum | 10 | >20 | >20 |
| Juncus effusus | 5 | 5 | 2.5 |
| Lasiosphaera fenzlii | 10 | 10 | >20 |
| Ligusticum chuanxiong | 2.5 | 5 | 5 |
| Lithospermum erythrorhizon | 0.3125 | 0.625 | 0.106 |
| Lonicera japonica | 5 | 5 | 5 |
| Morus alba | 0.625 | 5 | 2.5 |
| Nelumbo nucifera | 5 | 20 | >20 |
| Ophiopogon japonicus | 5 | 20 | 10 |
| Paeonia lactiflora | 5 | 10 | 2.5 |
| Paeonia suffruticosa | 5 | 10 | 10 |
| Phellodendron amurense | 2.5 | 5 | 5 |
| Polygonum cuspidatum | 10 | 2.5 | 2.5 |
| Prunella vulgaris | 2.5 | 1.25 | 10 |
| Prunus mume | 1.25 | 5 | 10 |
| Rhus chinensis | 1.25 | 1.25 | 1.25 |
| Sargassum fusiforme | 1.25 | 5 | 5 |
| Stemon japonica | 10 | 20 | 20 |
| Sophora flavescens | 20 | 20 | 10 |
| Syzygium aromaticum | 5 | 1.25 | 5 |
| Taraxacum mongolicum | 10 | 10 | 20 |
| Zanthoxylum bungeanum | 5 | 10 | 10 |
| Ligusticum chuanxiong, extract | 2.5 | 0.625 | 5 |
| Cnidium monnieri, extract | 10 | 10 | 10 |
| Alpinia japonica, extract | >10 | 2.5 | 5 |
| Rheum palmatum extract | 0.625 | 10 | 5 |
| Ginko extract | 5 | 2.5 | 2.5 |
| Puerarin extract | >10 | 5 | >10 |
| Forsythia suspensa, extract | >10 | >10 | >10 |
| Scutellaria baicalensis | 2.5 | 5 | 10 |
| Magnolia Bark Extract | 1.25 | 0.312 | 0.625 |
| Chlorohexdine Gluconate* | 0.005 | 0.01 | 0.01 |

The level of medicament or agent in the chewing gum formulation is selected so as to create, when the gum is chewed, a sufficiently high concentration of the medicament or agent in the saliva.

For comparison, when the agent is a stimulant such as nicotine or caffeine, the level of the stimulant in the chewing gum should be such that it creates a saliva content of stimulant of approximately 15 to 440 ppm when the chewing gum is chewed for 2 minutes. At this level, a sufficient amount of stimulant will be delivered to the chewer to create the effects set forth in the application. If a medicament is used such as a medicinal agent (e.g., analgesics), sufficient medicinal agent should be present in the chewing gum to create a salvia content of approximately 1700 to approximately 4400 ppm after the chewing gum has been chewed for 2 minutes. For botanical agents (e.g., chamomile, kava, kola, nut, ginseng, and Echinacea), the agent should be present in a sufficient amount to create a saliva content of approximately 85 to 1100 ppm when the chewing gum is chewed for 2 minutes. For a metabolizer, for example, chromium picolinate and hydroxi chitic acid, the agents should be present in an amount to create a saliva content of approximately 0.5 to about 900 ppm when chewed for at least two minutes. If the agent is a vitamin or mineral (e.g., phosphatidy serine, vitamin C, and zinc), the agent should be present in the amount to create a saliva content of the vitamin or mineral of approximately 10 to about 250 ppm when chewed for 2 minutes.

Pursuant to the present invention, depending on the agent or medicament, the dosing regiment will change. For example, if the medicament is an analgesic, the chewing gum would be taken on an as needed basis. Of course, similar to the oral administration of an analgesic, there would be restrictions on the number of pieces of chewing gum chewed, for example, not more often than one stick every four hours and not more often than four to five times a day. If the agent is a stimulant, comparable to caffeine to be used to enhance performance, then the chewing gum would be chewed, in a preferred embodiment ten minutes or less before the performance.

The medicament or agent can be contained in a variety of different chewing gum compositions. Referring now to the chewing gum, pursuant to the present invention, the chewing gum including the medicament or agent may be based on a variety of different chewing gums that are known. For example, the chewing gums can be low or high moisture, sugar or sugarless, wax containing or wax free, low calorie (via high base or low calorie bulking agents), and/or may contain dental agents.

Physical modifications of the active agent encapsulation with a highly water soluble substrate will increase its release in stick chewing gum as well as from the gum coating by increasing the solubility or dissolution rate. However, the active agent may also be encapsulated or entrapped to give a delayed release from stick chewing gum and from a gum coating. Any standard technique which gives partial or full encapsulation of the active agent can be used. These techniques include, but are not limited to, spray drying, spray chilling, fluid bed coating and coacervation. These encapsulation techniques may be used individually in a single step process or in any combination in a multiple step process.

Active agents may be encapsulated with sweeteners, more specifically high intensity sweeteners such as thaumatin, dihydrochalcones, acesulfame K, aspartame, N substituted APM derivatives such as neotame, sucralose, alitame, saccharin and cyclamates. These can also have the effect of reducing unpleasant tastes such as bitterness. Additional bitterness inhibitors or taste maskers can also be combined with active agents and sweeteners to give a reduced unpleasant taste such as bitterness with delayed release active agent(s).

The encapsulation techniques described herein are standard coating techniques and generally give varying degrees of coating from partial to full coating, depending on the coating composition used in the process. Generally, compositions that have high organic solubility, good film forming properties and low water solubility give better delayed release of active agents such as caffeine, while compositions that have high water solubility give better fast release. Such low water solubility compositions include acrylic polymers and copolymers, carboxyvinyl polymer, polyamides, polystyrene, polyvinyl acetate, polyvinyl acetate phthalate, polyvinylpyrrolidone and waxes. Although all of these materials are possible for encapsulation of active agents such as caffeine, only food grade materials should be considered. Two standard food grade coating materials that are good film formers but not water soluble are shellac and Zein. Others which are more water soluble, but good film formers, are materials like agar, alginates, a wide range of cellulose derivatives like ethyl cellulose, methyl cellulose, sodium hydroxymethyl cellulose, and hydroxypropylmethyl cellulose, dextrin, gelatin, and modified starches. These ingredients, which are generally approved for food use, may give a fast release when used as an encapsulant. Other encapsulants like acacia or maltodextrin can also encapsulate active agent(s) and give a fast release rate in gum.

The amount of coating or encapsulating material on the active agent also may control the length of time for its release from chewing gum. Generally, the higher the level of coating and the lower the amount of active agent, the slower the release during mastication with low water soluble compositions. The release rate is generally not instantaneous, but gradual over an extended period of time for stick gum. Delayed release allows the active agent to be masked in the mouth before being ingested, thus reducing bitterness or other unpleasant tastes. To obtain the delayed release, the encapsulant should be a minimum of about 20% of the coated active. Preferably, the encapsulant should be a minimum of about 30% of the coated active, and most preferably should be a minimum of about 40% of the coated active. Generally, water soluble encapsulating agents will increase the release rate of water insoluble active agents.

Another method of giving a modified release of active agent and the other agents described herein is agglomeration with an agglomerating agent which partially coats the active agents. This method includes the step of mixing active agents and an agglomerating agent with a small amount of water or solvent. The mixture is prepared in such a way as to have individual wet particles in contact with each other so that a partial coating can be applied. After the water or other solvent is removed, the mixture is ground and used as a powdered active agent.

Materials that can be used as the agglomerating agent are the same as those used in encapsulation mentioned previously. Some of the better agglomerating agents for delayed release are the organic polymers like acrylic polymers and copolymers, polyvinyl acetate, polyvinylpyrrolidone, waxes, shellac and Zein. Other agglomerating agents are not as effective in giving a delayed release as are the polymers, waxes, shellac and Zein, but can be used to give some delayed release. Other agglomerating agents include, but are not limited to, agar, alginates, a wide range of water soluble cellulose derivatives like ethyl cellulose, methyl cellulose, sodium hydroxymethyl cellulose, hydroxypropylmethyl cellulose, dextrin, gelatin, modified starches, and vegetable gums like guar gum, locust bean gum and carrageenan. Even though the agglomerated active, agent is only partially coated, when the quantity of coating is increased compared to the quantity of the active agent, the release can also be modified. The level of coating used in the agglomerated product is a minimum of about 5%. Preferably, the coating level is a minimum of about 15% and more preferably about 20%. Depending on the agglomerating agent, a higher or lower amount of agent may be needed to give the desired release of the active agent. Generally, water soluble agglomerants will increase the rate of release of water insoluble active agents.

Active agents may be coated in a two step process or a multiple step process. Active agents may be encapsulated with any of the materials as described previously and then the encapsulated caffeine or other active agents can be agglomerated as previously described to obtain an encapsulated/agglomerated active agent product that could be used in chewing gum to give a delayed release of the active agent.

In another embodiment of this invention, active agent may be absorbed onto another component which is porous and becomes entrapped in the matrix of the porous component. Common materials used for absorbing active agents include, but are not limited to, silicas, silicates, pharmasorb clay, sponge like beads or microbeads, amorphous carbonates and hydroxides, including aluminum and calcium lakes, all of which result in a delayed release of caffeine or other active agent.

Depending on the type of absorbent materials and how it is prepared, the amount of active agent that can be loaded onto the absorbent will vary. Generally materials like polymers or sponge like beads or microbeads, amorphous sugars and alditols and amorphous carbonates and hydroxides absorb about 10% to about 40% of the weight of the absorbent. Other materials like silicas and pharmasorb clays may be able to absorb about 20% to about 80% of the weight of the absorbent. Generally, water soluble absorbents will increase the release rate of water insoluble active agents.

The general procedure for absorbing active agent onto the absorbent is as follows. An absorbent like fumed silica powder can be mixed in a powder blender and a solution of active agent can be sprayed onto the powder as mixing continues. The aqueous solution can be about 1 to 2% solids, and higher solid levels to 15 30% may be used if temperatures up to 90° C. are used. Generally water is the solvent, but other solvents like alcohol could also be used if approved. As the powder mixes, the liquid is sprayed onto the powder. Spraying is stopped before the mix becomes damp. The still free flowing powder is removed from the mixer and dried to remove the water or other solvent, and is then ground to a specific particle size.

After the active agent is absorbed or fixed onto an absorbent, the fixative/active agent can be coated by encapsulation. Either full or partial encapsulation may be used, depending on the coating composition used in the process. Full encapsulation may be obtained by coating with a polymer as in spray drying, spray chilling, fluid bed coating, coapervation, or any other standard technique. A partial encapsulation or coating can be obtained by agglomeration of the fixative/active agent mixture using any of the materials discussed above.

Another form of encapsulation is by entrapment of an ingredient by fiber extrusion or fiber spinning into a polymer. Polymers that can be used for extrusion are PVAC, hydroxypropyl cellulose, polyethylene and other types of plastic polymers. A process of encapsulation by fiber extrusion is disclosed in U.S. Pat. No. 4,978,537, which is hereby incorporated by reference. The water insoluble polymer may be preblended with caffeine or other active agents prior to fiber extrusion, or may be added after the polymer is melted. As the extrudate is extruded, it results in small fibers that are cooled and ground. This type of encapsulation/entrapment generally gives a very long, delayed release of an active ingredient.

The four primary methods to obtain a treated active agent are: (1) encapsulation by spray drying, fluid bed coating, spray chilling and coacervation to give full or partial encapsulation, (2) agglomeration to give partial encapsulation, (3) fixation or absorption which also gives partial encapsulation, and (4) entrapment into an extruded compound. These four methods, combined in any usable manner which physically modifies active agents dissolvability or modifies the release of active agents, are included in this invention.

Medicament actives may be combined in a chewing gum. In a stick gum, two, three, or more actives may be added to a single piece. One active could be encapsulated for fast release, another active for moderate release, and another active for slow release. In addition, a single medicament active could be encapsulated and entrapped to release at various times as the gum is being chewed. This type of gum formulation could be effective for time release medication.

Medicament actives may also be combined in a coated chewing gum product. A single active may be added to a gum coating for fast release and also added to the gum center with or without encapsulation for slow release. If the active has an affinity for the gum base it may naturally give a slow release without encapsulation. If the active is fast release it would have to be encapsulated or entrapped for the desired time release.

In many instances a medicament may have a bitter taste. If the medicament were added to a coating at a very low level, it would still have the effect of fast release initially. In this case, the active agent may be added to the gum coating at a very low level beneath its taste threshold. Additional active agent that is encapsulated and entrapped may then be added to the gum center for slow release. This bitter active agent can then be kept below its taste threshold level and release slowly as the gum is being chewed, but the active agent would continue to be released to give its effective dosage.

In many instances, active medicaments may have a low quality off-taste or bitterness, especially if added to a chewing gum coating. In most cases, this off taste may be masked with high intensity sweeteners, but in other instances, a bitterness inhibitor may be needed to reduce a bitter taste of a medicament.

There are a wide variety of bitterness inhibitors that can be used in food products as well as with active agents. Some of the preferred bitterness inhibitors are the sodium salts which are discussed in the article Suppression of Bitterness by Sodium: Variations Among Bitter Taste Stimuli, by R. A. S. Breslin and G. K. Beceuchenp from Monell Chemical Senses Center, Philadelphia, Pa. Sodium salts discussed are sodium acetate and sodium gluconate. Other sodium salts that may also be effective are sodium glycinate, sodium ascorbate and sodium glycerophosphate. Among these, the most preferred is sodium gluconate and sodium glycinate since they have a low salty taste and are most effective to reduce bitterness of most active medicaments.

Most of the sodium salts are very water soluble and are readily released from chewing gum to function as bitterness inhibitors. In most instances, the sodium salts which release readily from chewing gum may be modified by encapsulation to give an even faster release from chewing gum. However, in some instances the sodium salts would be encapsulated or entrapped to give a delayed release from gum. Generally, the bitterness inhibitor should release with the active medicament for maximum effectiveness.

In addition to physically modifying the active medicament for fast or delayed release, medicaments may be dissolved in solvents, flavors, or other transdermal vehicles used as absorption enhancing agents and added to gum or to a gum coating. The absorption enhancing agents may also be added to the gum or gum coating separately from the active ingredient. Their presence may help volatilize medicaments or allow increased buccal/lingual absorption of the active agent through the nasal mucosal or the lungs. These solvents, flavors, or transdermal vehicles may transport medicaments faster through the oral mucosa.

Faster absorption may be affected by increasing flavor levels as well as the addition of other flavor components, such as menthol and menthol derivatives, limonene, carvone, isomenthol, eucalyptol, menthone, pinene, camphor and camphor derivatives, as well as monoterpene natural products, monoterpene derivatives, and sesquiterpenes, including caryophyllene and copaene. Other vehicles that may be used to increase transdermal absorption are: ethanol, polyethylene glycol, 2-pyrrolidones, myristic acid, Brij-35 (surfactant), p-phenyl phenol, nitrobenzene, stearyl alcohol, cetyl alcohol, croton oil, liquid paraffin, dimethyl sulfoxide (DMSO), non-ionic surfactants, liposomes, lecithin fractions, and long chain amphipathic molecules (molecules with polar or non-ionized groups on one end and non-polar groups at the other end).

In addition, some polysaccharides such as cellulose gums, natural gums like guar gum, gum arabic, and others may be mixed with active medicaments or mixed in the gum formulation with the medicament. This may allow the medicaments to stick to the surface of the oral mucosa during chewing and increase oral absorption. Bioadhesives may act in a similar manner to achieve increased absorption of the active medicament.

In some instances the gum formulation may have an effect on release rate of the medicament. Water miscible medicaments may be released more slowly when using a highly hydrophilic gum base and more quickly from a lipophilic gum base. On the other hand, oil miscible medicaments may release more quickly when using a highly hydrophilic gum base and more slowly from a lipophilic gum base. Also medicaments may release more quickly by using high HLB solubilizers in the gum formulation. Medicaments may also be emulsified together with water soluble bulking agents to increase release of the medicaments.

Other gum formula modifications may also affect the release rate of medicaments. Texture modifiers to soften base may give faster release where hard bases may give slower release. Addition of alkaline materials such as sodium bicarbonate or sodium hydroxide may make the saliva slightly alkaline, which may increase buccal/lingual absorption of the medicament into the bloodstream. Use of a buffer in the gum formula may affect release rate or absorption or shelf life of certain medicaments or supplements. Gum base made with talc may offer unique release and shelf life improvements. Other additives, such as astringents may give the sensation of dry mouth, which may improve medicament absorption. Also, some types of hot, spicy flavors such as ginger or hot pepper may give the impression of high activity of the medicament.

Medicaments may be added to chewing gum via special carriers which may affect the release rate and its absorption. Some carriers that may be used are activated charcoal, molecular sieves, corn starch granules, microsponges, or liposomes. The medicament may be sugar or polyol candy coated, or entrapped in cyclodextrin for fast release to dissolve quickly in the mouth during chewing.

Release of the medicament from gum may also be effected by particle size of the coated medicament. Small particles release more quickly whereas large particles more slowly. Fast release can also be accomplished by dissolving medicament in a liquid and used in a liquid center gum. Some medicaments may be advantageous to use in both slow and fast release. Quick release may give good oral absorption, then slow release may result by swallowing the cud. This may be particularly effective if a biodegradable gum base is used. On the other hand, some medicaments may have an advantage with a slow initial release, but increases later. This can reduce side effects of the medicament and improve adaptation to the medicament. Slow release may also be accomplished by attaching a medicament to a polymer used in the chewing gum.

Release of a medicament or active agent may also be effected by the shape and size of the chewing gum product. Flat stick pieces of gum with large surface area may release actives faster into saliva from gum when chewed, whereas round or cube pieces may release medicaments and actives more slowly. Gum formulations, especially those that are anhydrous or have no gum softening agents may be ground to a powder. This powder may be dusted onto the surface of another gum formulation or coated onto a ball or pillow shape gum product. This powder may also be tableted in a tablet press to give a unique form to be chewed for release of its active agent. Other forms of gum to be used are rolled sticks, or soft squeezable gum from a tube.

Active medicaments can also be added to chewing gum formulations that are made into tablets. Tableting of chewing gum is disclosed in U.K. Patent Publication No. 1,489,832; U.S. Pat. No. 4,753,805; EP Patent Publication No. 0 221 850; and Italy Patent Publication No. 1,273,487. These patents disclose active agents added to chewing gum which is then tableted. As an embodiment of this invention, active agents may be encapsulated or entrapped and added to a chewing gum formulation which is then tableted. In addition, a formed chewing gum tablet may also be used as a core for a coated chewing gum pellet that is coated with a sugar, polyol or film. The chewing gum core may contain one active agent or multiple active medicaments and the coating may contain one or more active medicaments. This form will yield unique chewing gum products.

The previously described encapsulated, agglomerated or absorbed active agent may readily be added to a chewing gum composition. The remainder of the chewing gum ingredients are well known to those of skill in the art and are not intended to be limiting to the present invention. That is, the treated particles of active agent can be added to conventional chewing gum formulations in a conventional manner. Treated active agent may be added to a sugar chewing gum or a sugarless chewing gum.

In general, a chewing gum composition typically comprises a water soluble bulk portion, a water insoluble chewable grams base portion and typically water insoluble flavoring agents. The water soluble portion dissipates with a portion of the flavoring agent over a period of time during chewing. The gum base portion is retained in the mouth throughout the chew.

The insoluble gum base generally comprises elastomers, resins, fats and oils, softeners and inorganic fillers. The gum base may or may not include wax. The insoluble gum base can constitute approximately 5% to about 95% by weight of the chewing gum, more commonly the gum base comprises 10% to about 50% of the gum, and in some preferred embodiments approximately 25% to about 35% by weight, of the chewing gum.

In a particular embodiment, the chewing gum base of the present invention contains about 20% to about 60% by weight synthetic elastomer, about 0% to about 30% by weight natural elastomer, about 5% to about 55% by weight elastomer plasticizer, about 4% to about 35% by weight filler, about 5% to about 35% by weight softener, and optional minor amounts (about 1% or less by weight) of miscellaneous ingredients such as colorants, antioxidants, etc.

Synthetic elastomers may include, but are not limited to, polyisobutylene with GPC weight average molecular weight of about 10,000 to about 95,000, isobutylene-isoprene copolymer (butyl elastomer), styrene butadiene, copolymers having styrene-butadiene ratios of about 1:3 to about 3:1, polyvinyl acetate having GPC weight average molecular weight of about 2,000 to about 90,000, polyisoprene, polyethylene, vinyl acetate vinyl laurate copolymer having vinyl laurate content of about 5% to about 50% by weight of the copolymer, and combinations thereof.

Preferred ranges for polyisobutylene are 50,000 to 80,000 GPC weight average molecular weight and for styrene butadiene are 1:1 to 1:3 bound styrene butadiene, for polyvinyl acetate are 10,000 to 65,000 GBC weight average molecular weight with the higher molecular weight polyvinyl acetates typically used in bubble gum base, and for vinyl acetate vinyl laurate, vinyl laurate content of 10 45%.

Natural elastomers may include natural rubber such as smoked or liquid latex and guayule as well as natural gums such as jelutong, lechi caspi, perillo, sorva, massaranduba balata, massaranduba chocolate, nispero, rosindinha, chicle, gutta hang kang, and combinations thereof. The preferred synthetic elastomer and natural elastomer concentrations vary depending on whether the chewing gum in which the base is used is adhesive or conventional, bubble gum or regular gum, as discussed below. Preferred natural elastomers include jelutong, chicle, sorva and massaranduba balata.

Elastomer plasticizers may include, but are not limited to, natural rosin esters such as glycerol esters or partially hydrogenated rosin, glycerol esters of polymerized rosin, glycerol esters of partially dimerized rosin, glycerol esters of rosin, pentaerythritol esters of partially hydrogenated rosin, methyl and partially hydrogenated methyl esters of rosin, pentaerythritol esters of rosin; synthetics such as terpene resins derived from alpha pinene, beta pinene, and/or d limonene; and any suitable combinations of the foregoing. The preferred elastomer plasticizers will also vary depending on the specific application, and on the type of elastomer which is used.

Fillers/texturizers may include magnesium and calcium carbonate, ground limestone, silicate types such as magnesium and aluminum silicate, clay, alumina, talc, titanium oxide, mono, di- and tri-calcium phosphate, cellulose polymers, such as wood, and combinations thereof.

Softeners/emulsifiers may include tallow, hydrogenated tallow, hydrogenated and partially hydrogenated vegetable oils, cocoa butter, glycerol monostearate, glycerol triacetate, lecithin, mono, di- and triglycerides, acetylated monoglycerides, fatty acids (e.g. stearic, palmitic, oleic and linoleic acids), and combinations thereof.

Colorants and whiteners may include FD&C-type dyes and lakes, fruit and vegetable extracts, titanium dioxide, and combinations thereof.

The base may or may not include wax. An example of a wax free gum base is disclosed in U.S. Pat. No. 5,286,500, the disclosure of which is incorporated herein by reference.

In addition to a water insoluble gum base portion, a typical chewing gum composition includes a water soluble bulk portion and one or more flavoring agents. The water soluble portion can include bulk sweeteners, high intensity sweeteners, flavoring agents, softeners, emulsifiers, colors, acidulants, fillers, antioxidants, and other components that provide desired attributes.

Softeners are added to the chewing gum in order to optimize the chewability and mouth feel of the gum. The softeners, which are also known as plasticizers and plasticizing agents, generally constitute between approximately 0.5% to about 15% by weight of the chewing gum. The softeners may include glycerin, lecithin, and combinations thereof. Aqueous sweetener solutions such as those containing sorbitol, hydrogenated starch hydrolysates, corn syrup and combinations thereof, may also be used as softeners and binding agents in chewing gum.

Bulk sweeteners include both sugar and sugarless components. Bulk sweeteners typically constitute about 5% to about 95% by weight of the chewing gum, more typically, about 20% to about 80% by weight, and more commonly, about 30% to about 60% by weight of the gum. Sugar sweeteners generally include saccharide containing components commonly known in the chewing gum art, including but not limited to, sucrose, dextrose, maltose, dextrin, dried invert sugar, fructose, levulose, galactose, corn syrup solids, and the like, alone or in combination. Sugarless sweeteners include, but are not limited to, sugar alcohols such as sorbitol, mannitol, xylitol, hydrogenated starch hydrolysates, maltitol, and the like, alone or in combination.

High intensity artificial sweeteners can also be used, alone or in combination, with the above. Preferred sweeteners include, but are not limited to, sucralose, aspartame, N substituted APM derivatives such as neotame, salts of acesulfame, altitame, saccharin and its salts, cyclamic acid and its salts, glycyrrhizinate, dihydrochalcones, thaumatin, monellin, and the like, alone or in combination. In order to provide longer lasting sweetness and flavor perception, it may be desirable to encapsulate or otherwise control the release of at least a portion of the artificial sweetener. Such techniques as wet granulation, wax granulation, spray drying, spray chilling, fluid bed coating, coacervation, and fiber extension may be used to achieve the desired release characteristics.

Combinations of sugar and/or sugarless sweeteners may be used in chewing gum. Additionally, the softener may also provide additional sweetness such as with aqueous sugar or alditol solutions.

If a low calorie gum is desired, a low caloric bulking agent can be used. Examples of low caloric bulking agents include: polydextrose; Raftilose, Raftilin; Fructooligosaccharides (NutraFlora); Palatinose oligosaccharide; Guar Gum Hydrolysate (Sun Fiber); or indigestible dextrin (Fibersol). However, other low calorie bulking agents can be used.

A variety of flavoring agents can also be used, if desired. The flavor can be used in amounts of about 0.1 to about 15 weight percent of the gum, and preferably, about 0.2% to about 5% by weight. Flavoring agents may include essential oils, synthetic flavors or mixtures thereof including, but not limited to, oils derived from plants and fruits such as citrus oils, fruit essences, peppermint oil, spearmint oil, other mint oils, clove oil, oil of wintergreen, anise and the like. Artificial flavoring agents and components may also be used. Natural and artificial flavoring agents may be combined in any sensorially acceptable fashion.

If the medicament or active is water soluble in the chewing gum, it preferably will include a base/emulsifier system which leads to the desired concentration of the medicament in the saliva (more hydrophilic balance). If the medicament or active is water insoluble, the chewing gum preferably includes a base/emulsifier system which leads to the desired concentration of the medicament in the saliva (more lipophilic balance).

In manufacturing the chewing gum including the active agent or ingredient, the active agent or medicament is added, preferably, early on in the mix. The smaller the amount of active ingredient used, the more necessary it becomes to preblend that particular ingredient to assume uniform distribution throughout the batch of gum. Whether a preblend is used or not, the active agent or medicament should be added within the first five minutes of mixing. For faster release, the active agent may be added late in the process.

In general, chewing gum is manufactured by sequentially adding the various chewing gum ingredients to a commercially available mixer known in the art. After the ingredients have been thoroughly mixed, the gum mass is discharged from the mixer and shaped into the desired form such as rolling sheets and cutting into sticks, extruding into chunks or casting into pellets, which are then coated or panned.

Generally, the ingredients are mixed by first melting the gum base and adding it to the running mixer. The base may also be melted in the mixer itself. Color or emulsifiers may also be added at this time. A softener such as glycerin may also be added at this time, along with syrup and a portion of the bulking agent. Further parts of the bulking agent are added to the mixer. Flavoring agents are typically added with the final portion of the bulking agent. Other optional ingredients are added to the batch in a typical fashion, well known to those of ordinary skill in the art.

The entire mixing procedure typically takes from five to fifteen minutes, but longer mixing times may sometimes be required. Those skilled in the art will recognize that many variations of the above described procedure may be followed.

Chewing gum base and chewing gum product have been manufactured conventionally using separate mixers, different mixing technologies and, often, at different factories. One reason for this is that the optimum conditions for manufacturing gum base, and for manufacturing chewing gum from gum base and other ingredients such as sweeteners and flavors, are so different that it has been impractical to integrate both tasks. Chewing gum base manufacture, on the one hand, involves the dispersive (often high shear) mixing of difficult-to-blend ingredients such as elastomer, filler, elastomer plasticizer, base softeners/emulsifiers and sometimes wax, and typically requires long mixing times. Chewing gum product manufacture, on the other hand, involves combining the gum base with more delicate ingredients such as product softeners, bulk sweeteners, high intensity sweeteners and flavoring agents using distributive (generally lower shear) mixing, for shorter periods.

In order to improve the efficiency of gum base and gum product manufacture, there has been a trend toward the continuous manufacture of gum bases and products. U.S. Pat. No. 3,995,064, issued to Ehrgoft et al., discloses the continuous manufacture of gum base using a sequence of mixers or a single variable mixer. U.S. Pat. No. 4,459,311, issued to DeTora et al., also discloses the continuous manufacture of gum base using a sequence of mixers. Other continuous gum base manufacturing processes are disclosed in European Publication No. 0,273,809 (General Foods France) and in French Publication No. 2,635,441 (General Foods France).

U.S. Pat. No. 5,045,325, issued to Lesko et al., and U.S. Pat. No. 4,555,407, issued to Kramer et al., disclose processes for the continuous production of chewing gum products. In each case, however, the gum base is initially prepared separately and is simply added into the process. U.S. Pat. No. 4,968,511, issued to D'Amelia et al., discloses a chewing gum product containing certain vinyl polymers which can be produced in a direct one-step process not requiring separate manufacture of gum base.

Active medicaments may also be added to chewing gum products made by a continuous process. U.S. Pat. Nos. 5,543,160 and 5,800,847 disclose a continuous process using a single extruder to make the gum base and the gum product. U.S. Pat. Nos. 5,397,580 and 5,523,097 disclose a continuous process using two or more extruders for base and chewing gum mixing. U.S. Pat. Nos. 5,419,919 and 5,571,543 disclose a continuous process using a paddle type mixer which has low pressure and high residence time for adequate mixing.

Active medicaments, whether encapsulated, entrapped or not, can be added at any time during the continuous mixing process. Generally, actives would probably be added in the gum mixing sections. Specific advantages to adding active medicaments to a continuous process of manufacturing gum are that more thorough mixing is accomplished in this type of process with lower amount of residence time of the active agent at high temperatures during processing. The enclosed system used in continuous processing can result in more thorough mixing, better reproducibility of the amount of active within the gum matrix, and less loss in the amount of the active medicament.

Another method of treating the medicament or active agent is to physically isolate the active agent from other chewing gum ingredients to effect its release rate and stability. The active agent may be added to the liquid inside a liquid center gum product. The center fill of gum product may comprise one or more carbohydrate syrups, glycerin, thickeners, flavors, acidulants, colors, sugars and sugar alcohols in conventional amounts. The ingredients are combined in a conventional manner. The total amount of active agent may be dissolved in the center fill liquid. This method of using active agent in chewing gum may give a more controlled release rate, and may reduce or eliminate any possible reaction with gum base, flavor components, or other components, yielding improved shelf stability. A liquid-center gum may also be coated with a sugar, polyol or film to yield a unique chewing gum product.

Another method of isolating medicaments or active agents from other chewing gum ingredients is to add active agents to the dusting compound of a chewing gum. A rolling or dusting compound serves to reduce sticking to machinery as it is wrapped, and sticking to its wrapper after it is wrapped and being stored. The rolling compound comprises active agents in combination with mannitol, sorbitol, sucrose, starch, calcium carbonate, talc, other orally acceptable substances or a combination thereof. The rolling compound constitutes from about 0.25% to about 10.0% or about 1% to about 3% of weight of the chewing gum composition. This method of using active agents in the chewing gum can allow a lower usage level, can give a more controlled release rate, and can reduce or eliminate any possible reaction with the gum base, flavor components, or other components, yielding improved self stability.

Another method of isolating medicament or active agents is to use it in the coating/panning of a pellet chewing gum. Pellet or ball gum is prepared as conventional chewing gum but formed into pellets that are pillow shaped, or into balls. The pellets/balls can be then sugar coated or panned by conventional panning techniques to make a unique coated pellet gum. The active agent may be soluble in flavor or can be blended with other powders often used in some types of conventional panning procedures. Active agents are isolated from other gum ingredients which modifies its release rate from chewing gum. Levels of actives may be about 10 ppm to 5% by weight of chewing gum coating. The weight of the coating may be about 20% to about 50% of the weight of the finished product, but may be as much as 75% of the total gum product.

Conventional panning procedures generally coat with sucrose, but recent advances in panning have allowed use of other carbohydrate materials to be used in place of sucrose. Some of these components include, but are not limited to, dextrose, maltose, palatinose, xylitol, lactitol, hydrogenated isomaltulose, erythritol maltitol, and other new alditols or combinations thereof. These materials may be blended with panning modifiers including, but not limited to, gum arabic, maltodextrins, corn syrup, gelatin, cellulose type materials like carboxymethyl cellulose or hydroxymethyl cellulose, starch and modified starches, vegetables gums like alginates, locust bean gum, guar gum, and gum tragacanth, insoluble carbonates like calcium carbonate or magnesium carbonate and talc. Antitack agents may also be added as panning modifiers, which allow the use of a variety of carbohydrates and sugar alcohols to be used in the development of new panned or coated gum products. Flavors may also be added with the sugar or sugarless coating and with the active to yield unique product characteristics.

Another type of pan coating could also isolate the active agent from the chewing gum ingredients. This technique is referred to as a film coating and is more common for pharmaceuticals than in chewing gum, but procedures are similar. A film like shellac, zein, or cellulose type material is applied onto a pellet type product forming a thin film on the surface of the product. The film is applied by mixing the polymer, plasticizer and a solvent (pigments are optional) and spraying the mixture onto the pellet surface. This is done in conventional type panning equipment, or in more advanced side vended coating pans. Since most active agents may be alcohol soluble, they may be readily added with this type of film. When a solvent like an alcohol is used, extra precautions are needed to prevent fires and explosions, and specialized equipment must be used.

Some film polymers can use water as the solvent in film coating. Recent advances in polymer research and in film coating technology eliminates the problem associated with the use of solvents in coating. These advances make it possible to apply aqueous films to a pellet or chewing gum product. Some active agents can be added to this aqueous film or even the alcohol solvent film, in which an active agent is highly soluble. This film may also contain a flavor along with a polymer and plasticizer. The active agent can also be dissolved in the aqueous solvent and coated on the surface with the aqueous film. This will give a unique sweetness release to a film coated product.

After a coating film with an active medicament is applied to a chewing gum product, a hard shell sugar or polyol coating may then be applied over the film coated product. In some instances a soft shell sugar or polyol coating may also be used over the film coated product. The level of film coating applied to a pellet gum may be generally about 0.5% to about 3% of the gum product. The level of overcoating of the hard or soft shell may be about 20% to about 60%. When the active agent is added with the film coating and not with the sugar/polyol coating, better control of the amount of active agent in the product may be obtained. In addition, the sugar/polyol overcoating may give an improved stability to the active agent in the product.

As noted above, the coating may contain ingredients such as flavoring agents, as well as artificial sweeteners and dispersing agents, coloring agents, film formers and binding agents. Flavoring agents contemplated by the present invention include those commonly known in the art such as essential oils, synthetic flavors or mixtures thereof, including but not limited to oils derived from plants and fruits such as citrus oils, fruit essences, peppermint oil, spearmint oil, other mint oils, clove oil, oil of wintergreen, anise and the like. The flavoring agents may be used in an amount such that the coating will contain from about 0.2% to about 3% flavoring agent, and preferably from about 0.7% to about 2.0% flavoring agent.

Artificial sweeteners contemplated for use in the coating include but are not limited to synthetic substances, saccharin, thaumatin, alitame, saccharin salts, aspartame, N substituted APM derivatives such as neotame, sucralose and acesulfame-K. The artificial sweetener may be added to the coating syrup in an amount such that the coating will contain from about 0.01% to about 0.5%, and preferably from about 0.1% to about 0.3% artificial sweetener.

Dispersing agents are often added to syrup coatings for the purpose of whitening and tack reduction. Dispersing agents contemplated by the present invention to be employed in the coating syrup include titanium dioxide, talc, or any other antistick compound. Titanium dioxide is a presently preferred dispersing agent of the present invention. The dispersing agent may be added to the coating syrup in amounts such that the coating will contain from about 0.1% to about 1.0%, and preferably from about 0.3% to about 0.6% of the agent.

Coloring agents are preferably added directly to the syrup in the dye or lake form. Coloring agents contemplated by the present invention include food quality dyes. Film formers preferably added to the syrup include methyl cellulose, gelatins, hydroxypropyl cellulose, ethyl cellulose, hydroxyethyl cellulose, carboxymethyl cellulose and the like and combinations thereof. Binding agents may be added either as an initial coating on the chewing gum center or may be added directly into the syrup. Binding agents contemplated by the present invention include gum arabic, gum talha (another type of acacia), alginate, cellulosics, vegetable gums and the like.

The coating is initially present as a liquid syrup which contains from about 30% to about 80% or 85% of the coating ingredients previously described herein, and from about 15% or 20% to about 70% of a solvent such as water. In general, the coating process is carried out in a rotating pan. Sugar or sugarless gum center tablets to be coated are placed into the rotating pan to form a moving mass.

The material or syrup which will eventually form the coating is applied or distributed over the gum center tablets. Flavoring agents may be added before, during and after applying the syrup to the gum centers. Once the coating has dried to form a hard surface, additional syrup additions can be made to produce a plurality of coatings or multiple layers of hard coating.

In a hard coating panning procedure, syrup is added to the gum center tablets at a temperature range of from about 100° F. to about 240° F. Preferably, the syrup temperature is from about 130° F. to about 200° F. throughout the process in order to prevent the polyol or sugar in the syrup from crystallizing. The syrup may be mixed with, sprayed upon, poured over, or added to the gum center tablets in any way known to those skilled in the art.

In general, a plurality of layers is obtained by applying single coats, allowing the layers to dry, and then repeating the process. The amount of solids added by each coating step depends chiefly on the concentration of the coating syrup. Any number of coats may be applied to the gum center tablet. Preferably, no more than about 75-100 coats are applied to the gum center tablets. The present invention contemplates applying an amount of syrup sufficient to yield a coated comestible containing about 10% to about 65% coating. Where higher dosage of an active agent is needed, the final product may be higher than 65% coating.

Those skilled in the art will recognize that in order to obtain a plurality of coated layers, a plurality of premeasured aliquots of coating syrup may be applied to the gum center tablets. It is contemplated, however, that the volume of aliquots of syrup applied to the gum center tablets may vary throughout the coating procedure.

Once a coating of syrup is applied to the gum center tablets, the present invention contemplates drying the wet syrup in an inert medium. A preferred drying medium comprises air. Preferably, forced drying air contacts the wet syrup coating in a temperature range of from about 70° to about 115° F. More preferably, the drying air is in the temperature range of from about 80° to about 100° F. The invention also contemplates that the drying air possess a relative humidity of less than about 15 percent. Preferably, the relative humidity of the drying air is less than about 8 percent.

The drying air may be passed over and admixed with the syrup coated gum centers in any way commonly known in the art. Preferably, the drying air is blown over and around or through the bed of the syrup coated gum centers at a flow rate, for large scale operations, of about 2800 cubic feet per minute. If lower quantities of material are being processed, or if smaller equipment is used, lower flow rates would be used.

For many years, flavors have been added to a sugar coating of pellet gum to enhance the overall flavor of gum. These flavors include spearmint flavor, peppermint flavor, wintergreen flavor, and fruit flavors. These flavors are generally preblended with the coating syrup just prior to applying it to the core or added together to the core in one or more coating applications in a revolving pan containing the cores. Generally, the coating syrup is very hot, about 130° to 200° F., and the flavor may volatilize if preblended with the coating syrup too early.

The concentrated coating syrup is applied to the gum cores as a hot liquid, the sugar or polyol allowed to crystallize, and the coating then dried with warm, dry air. This is repeated in about 30 to 80 applications to obtain a hard shell coated product having an increased weight gain of about 40% to 75%. A flavor is applied with one, two, three or even four or more of these coating applications. Each time flavor is added, several non-flavored coatings are applied to cover the flavor before the next flavor coat is applied. This reduces volatilization of the flavor during the coating process.

For mint flavors such spearmint, peppermint and wintergreen, some of the flavor components are volatilized, but sufficient flavor remains to give a product having a strong, high impact flavor. Fruit flavors, that may contain esters, are more easily volatilized and may be flammable and/or explosive and therefore, generally these type of fruit flavors are not used in coatings.

In an embodiment of this invention, an active agent is preblended with a gum arabic solution to become a paste and then applied to the cores. To reduce stickiness, the preblend may be mixed with a small amount of coating syrup before being applied. Forced air drying is then continued as the gum arabic binds the active agent to the cores. Then additional coatings are applied to cover the active agent and imbed the treated active agent in the coatings.

### GUM FORMULATION EXAMPLES

The following examples of the invention and comparative examples are provided by way of explanation and illustration.

As noted earlier, the gum formulas can be prepared as stick or tab products in the sugar or sugarless type formulations. These formulas can also be made in a pellet or pillow shape pellet or a round ball or any other shape of product for coating/panning. However, gum formulas for pellet centers are generally adjusted to a higher level of gum base to give a more consumer acceptable size of gum bolus.

Keeping this in mind, if a coating of about 25% of the total product is added to a pellet core as sugar or polyols, the gum base in the pellet core should also be increased by 25%. Likewise, if a 33% coating is applied, the base levels should also be increased by 33%. As a result, gum centers are usually formulated with about 25% to about 40% gum base with a corresponding decrease in the other ingredients except flavor. Even higher levels of base may be used if an active is added to a pellet coating. Generally flavors increase with the level of gum base as the base tends to bind flavors into the gum and more flavor is needed to give a good flavorful product. However flavors can also be added to the coating to give increased flavor impact and more flavor perception.

A wide range of changes and modifications to the embodiments of the invention described above will be apparent to persons skilled in the art. For example, while the invention is described with respect to hard-coated chewing gum, it will be appreciated that the process is applicable to coating other food products, such as candies, in which a coating with Wolfberry extract would have utility.

### Chewing Gum Examples

The following examples of the invention and comparative examples are provided by way of explanation and illustration.

The formulas listed in Table 1 comprise various sugar-type formulas in which active medicament can be added to gum after it is dissolved in water or mixed with various aqueous solvents. Wolfberry extract is an active medicament used as an oral anesthetic for sore throat. These formulas give a 3 gram stick with 3 mg of Wolfberry extract.

TABLE 1
(WEIGHT PERCENT)

| | EX. 1 | EX. 2 | EX. 3 | EX. 4 | EX. 5 | EX. 6 | EX. 7 | EX. 8 |
|---|---|---|---|---|---|---|---|---|
| SUGAR | 62.5 | 64.6 | 63.6 | 65.0 | 65.0 | 63.0 | 61.6 | 47.0 |
| BASE | 19.2 | 19.2 | 19.2 | 19.2 | 19.2 | 19.2 | 19.2 | 19.0 |
| CORN SYRUP | 15.9 | 12.9 | 12.9 | 12.9 | 12.9 | 15.9 | 0.0 | 2.9 |
| COOLING | 0.1 | — | — | — | 0.2 | — | — | 0.2 |
| AGENT | | | | | | | | |
| PEPPERMINT | 0.8 | 0.9 | 0.9 | 0.9 | 0.7 | 0.0 | 0.9 | 0.9 |
| FLAVOR | | | | | | | | |
| GLYCERIN | 1.4 | 1.4 | 1.4 | 0.0 | 0.0 | 0.9 | 1.4 | 0.0 |
| LIQUID/ | 0.1 | 1.0 | 2.0 | 2.0 | 2.0 | 1.0 | 16.9 | 30.0 |
| ACTIVE | | | | | | | | |
| BLEND | | | | | | | | |

### Example 1

Wolfberry extract powder can be added directly to the gum.

### Example 2

A 1 gram quantity of Wolfberry extract can be dissolved in 9 grams of water giving a 10% solution and added to gum.

### Example 3

A 1 gram quantity of Wolfberry extract can be dissolved in 9 grams of water and mixed with 10 grams of glycerin and added to the gum.

### Example 4

A 1 gram quantity of Wolfberry extract is mixed with 19 grams of glycerin giving a 5% solution and added to gum.

### Example 5

A 1 gram quantity of Wolfberry extract is mixed with 19 grams of propylene glycol giving a 5% solution and added to gum.

### Example 6

A 1 gram quantity of Wolfberry extract is dissolved in 9 grams of ethanol, which is then mixed with 90 grams of peppermint flavor and added to gum.

### Example 7

A 1 gram quantity of Wolfberry extract is dissolved in 168 grams of corn syrup and added to chewing gum.

### Example 8

To a 200 gram quantity of corn syrup is added 100 grams of glycerin. To this mixture is added 1 gram of Wolfberry extract and blended. This mixture is then added to gum.

In the next examples of sugar formulations, Wolfberry extract can be dissolved in water and emulsifiers can be added to the aqueous solution. Example solutions can be prepared by dissolving 10 grams of Wolfberry extract in 75 grams of water and adding 15 grams of emulsifiers of various hydrophilic-lipophilic balance (HLB) values to the solution. The mixtures can then be used in the following formulas. Example 9 uses a mixture of Wolfberry extract and water with no emulsifier. The HLB value of the emulsifiers used in Examples 10-14 are listed in Table 2.

TABLE 2
(WEIGHT PERCENT)

| | EX. 9 | EX. 10 | EX. 11 | EX. 12 | EX. 13 | EX. 14 |
|---|---|---|---|---|---|---|
| SUGAR | 54.7 | 54.7 | 54.7 | 54.7 | 54.7 | 54.7 |
| BASE | 19.2 | 19.2 | 19.2 | 19.2 | 19.2 | 19.2 |
| CORN SYRUP | 12.9 | 12.9 | 12.9 | 12.9 | 12.9 | 12.9 |
| GLYCERIN | 1.4 | 1.4 | 1.4 | 1.4 | 1.4 | 1.4 |
| DEXTROSE | 9.9 | 9.9 | 9.9 | 9.9 | 9.9 | 9.9 |
| MONOHYDRATE | | | | | | |
| FLAVOR | 0.9 | 0.9 | 0.9 | 0.9 | 0.9 | 0.9 |
| ACTIVE AGENT EMULSIFIER/ | 1.0 | 1.0 | 1.0 | 1.0 | 1.0 | 1.0 |
| WATER MIXTURE | | | | | | |
| | None | HLB = 2 | HLB = 4 | HLB = 6 | HLB = 9 | HLB = 12 |

### Examples 15-20

The same as the formulations made in Examples 9-14, respectively, except that the flavor can be mixed together with the aqueous Wolfberry extract solution and emulsified before adding the mixture to the gum batch.

The following Tables 3 through 10 are examples of gum formulations that demonstrate formula variations in which Wolfberry extract may be used. The active agent may be added with or without encapsulation, or may be treated for fast release.

Examples 21-24 in Table 3 demonstrates the use of Wolfberry extract in low-moisture sugar formulations showing less than 2% theoretical moisture:

TABLE 3
(WEIGHT PERCENT)

| | EX. 21 | EX. 22 | EX. 23 | EX. 24 |
|---|---|---|---|---|
| SUGAR | 58.8 | 58.6 | 58.8 | 54.6 |
| GUM BASE | 19.2 | 19.2 | 19.2 | 19.2 |
| CORN^{a)} SYRUP | 6.0 | 6.0 | — | — |
| DEXTROSE | 10.0 | 10.0 | 10.0 | 10.0 |
| MONOHYDRATE | | | | |
| LACTOSE | 0.0 | 0.0 | 0.0 | 5.0 |
| GLYCERIN^{b)} | 5.0 | 5.0 | 11.0 | 10.0 |
| FLAVOR | 0.9 | 0.9 | 0.9 | 0.9 |
| WOLFBERRY EXTRACT | 0.1 | 0.3 | 0.1 | 0.3 |

^{a)}Corn syrup is evaporated to 85% solids, 15% moisture
^{b)}Glycerin and syrup may be blended and co-evaporated

Examples 25-28 in Table 4 demonstrate the use of Wolfberry extract in medium-moisture sugar formulations having about 2% to about 5% moisture.

Examples 29-32 in Table 5 demonstrate the use of Wolfberry extract in high-moisture sugar formulations having more than about 5% moisture.

TABLE 4
(WEIGHT PERCENT)

| | EX. 25 | EX. 26 | EX. 27 | EX. 25 |
|---|---|---|---|---|
| SUGAR | 53.4 | 53.2 | 53.4 | 49.7 |
| GUM BASE | 19.2 | 19.2 | 19.2 | 19.2 |
| CORN SYRUP^{a)} | 15.0 | 15.0 | 13.0 | 12.5 |
| DEXTROSE MONOHYDRATE | 10.0 | 10.0 | 10.0 | 10.0 |
| GLYCERIN^{b)} | 1.4 | 1.4 | 3.4 | 7.4 |
| FLAVOR | 0.9 | 0.9 | 0.9 | 0.9 |
| WOLFBERRY EXTRACT | 0.1 | 0.3 | 0.1 | 0.3 |

^{a)}Corn syrup is evaporated to 85% solids, 15% moisture
^{b)}Glycerin and syrup may be blended and co-evaporated

TABLE 5
(WEIGHT PERCENT)

| | EX. 29 | EX. 30 | EX. 31 | EX. 32 |
|---|---|---|---|---|
| SUGAR | 50.8 | 50.7 | 49.8 | 49.7 |
| GUM BASE | 24.0 | 24.0 | 24.0 | 24.0 |
| CORN SYRUP | 24.0 | 24.0 | 24.0 | 24.6 |
| GLYCERIN | — | — | 1.0 | 0.4 |
| COOLING AGENT | 0.1 | — | 0.1 | — |
| FLAVOR | 1.0 | 1.0 | 1.0 | 1.0 |
| WOLFBERRY EXTRACT | 0.1 | 0.3 | 0.1 | 0.3 |

Examples 33-36 in Table 6 and Examples 37-44 in Tables 7 and 8 demonstrate the use of Wolfberry extract in low- and high-moisture gums that are sugar-free. Low-moisture gums have less than about 2% moisture, and high-moisture gums have greater than 2% moisture.

TABLE 6
(WEIGHT PERCENT)

| | EX. 33 | EX. 34 | EX. 35 | EX. 36 |
|---|---|---|---|---|
| BASE | 25.5 | 25.5 | 25.5 | 25.5 |
| SORBITOL | 50.9 | 50.7 | 48.9 | 45.7 |
| MANNITOL | 12.0 | 12.0 | 12.0 | 12.0 |
| GLYCERIN | 10.0 | 10.0 | 12.0 | 15.0 |
| FLAVOR | 1.5 | 1.5 | 1.5 | 1.5 |
| WOLFBERRY EXTRACT | 0.1 | 0.3 | 0.1 | 0.3 |

TABLE 7
(WEIGHT PERCENT)

| | EX. 37 | EX. 38 | EX. 39 | EX. 40 |
|---|---|---|---|---|
| BASE | 25.5 | 25.5 | 25.5 | 25.5 |
| SORBITOL | 50.9 | 50.7 | 40.9 | 40.7 |
| LIQUID SORBITOL* | 10.0 | 10.0 | 20.0 | 20.0 |
| MANNITOL | 10.0 | 10.0 | 10.0 | 10.0 |
| GLYCERIN | 2.0 | 2.0 | 2.0 | 2.0 |
| FLAVOR | 1.5 | 1.5 | 1.5 | 1.5 |
| WOLFBERRY EXTRACT | 0.1 | 0.3 | 0.1 | 0.3 |

*Sorbitol liquid contains 70% sorbitol, 30% water

TABLE 8
(WEIGHT PERCENT)

| | EX. 41 | EX. 42 | EX. 43 | EX. 44 |
|---|---|---|---|---|
| BASE | 25.5 | 25.5 | 25.5 | 25.5 |
| SORBITOL | 50.9 | 48.7 | 44.9 | 42.7 |
| HSH SYRUP* | 10.0 | 10.0 | 10.0 | 10.0 |
| MANNITOL | 8.0 | 8.0 | 8.0 | 8.0 |
| GLYCERIN** | 4.0 | 6.0 | 10.0 | 12.0 |
| FLAVOR | 1.5 | 1.5 | 1.5 | 1.5 |
| WOLFBERRY EXTRACT | 0.1 | 0.3 | 0.1 | 0.3 |

*Hydrogenated starch hydrolyzate syrup
**Glycerin and HSH syrup may be blended or co-evaporated

Table 9 shows sugar chewing formulations that can be made with various types of sugars.

TABLE 9
(WEIGHT PERCENT)

| | EX. 45 | EX. 46 | EX. 47 | EX. 48 | EX. 49 | EX. 50 |
|---|---|---|---|---|---|---|
| GUM BASE | 19.1 | 19.2 | 19.2 | 19.2 | 19.2 | 19.1 |
| SUCROSE | 49.4 | 48.2 | 44.4 | 39.2 | 34.4 | 42.2 |
| GLYCERIN | 1.4 | 2.4 | 1.4 | 6.4 | 1.4 | 3.4 |
| CORN SYRUP | 14.0 | 14.0 | 14.0 | 14.0 | 14.0 | 14.0 |
| DEXTROSE | 5.0 | 5.0 | — | — | 10.0 | 5.0 |
| LACTOSE | 5.0 | 5.0 | 10.0 | 10.0 | — | — |
| FRUCTOSE | 5.0 | 5.0 | 10.0 | 10.0 | 10.0 | 4.0 |
| INVERT | — | — | — | — | 10.0 | 10.0 |
| SUGAR | | | | | | |
| MALTOSE | — | — | — | — | — | 1.0 |
| COOLING | 0.1 | — | — | — | — | 0.1 |
| AGENT | | | | | | |
| FRUIT | — | 0.9 | — | 0.2 | — | — |
| FLAVOR | | | | | | |
| MINT | 0.9 | — | 0.9 | 0.7 | 0.9 | 0.9 |
| FLAVOR | | | | | | |
| ACTIVE | 0.1 | 0.3 | 0.1 | 0.3 | 0.1 | 0.3 |
| WOLFBERRY | | | | | | |
| EXTRACT | | | | | | |
| | EX. 51 | EX. 52 | EX. 53 | EX. 54 | EX. 55 | EX. 56 |
| GUM BASE | 19.2 | 19.2 | 19.2 | 19.2 | 19.2 | 19.2 |
| SUCROSE | 34.4 | 43.2 | 34.4 | 43.2 | 42.4 | 45.2 |
| GLYCERIN | 1.4 | 2.4 | 1.4 | 2.4 | 1.4 | 3.4 |
| CORN SYRUP | 14.0 | 14.0 | 14.0 | 14.0 | 11.0 | 11.0 |
| DEXTROSE | 10.0 | 5.0 | 10.0 | 5.0 | 10.0 | 5.0 |
| LACTOSE | — | — | — | — | — | — |
| FRUCTOSE | 10.0 | 5.0 | 10.0 | 5.0 | 5.0 | 5.0 |
| INVERT | 10.0 | 10.0 | — | — | 5.0 | 5.0 |
| SUGAR | | | | | | |
| MALTOSE | — | — | 10.0 | 10.0 | — | — |
| CORN SYRUP | — | — | — | — | 5.0 | 5.0 |
| SOLIDS | | | | | | |
| PEPPERMINT | 0.9 | 0.9 | 0.9 | 0.9 | 0.9 | 0.9 |
| FLAVOR | | | | | | |
| ACTIVE | 0.1 | 0.3 | 0.1 | 0.3 | 0.1 | 0.3 |
| WOLFBERRY | | | | | | |
| EXTRACT | | | | | | |

Table 10 shows chewing gum formulations that are free of sugar. These formulations can use a wide variety of other non-sugar alditols.

TABLE 10
(WEIGHT PERCENT)

| | EX. 57 | EX. 58 | EX. 59 | EX. 60 | EX. 61 | EX. 62 |
|---|---|---|---|---|---|---|
| GUM BASE | 25.5 | 25.5 | 25.5 | 25.5 | 25.5 | 25.5 |
| GLYCERIN | 8.0 | 8.0 | 8.0 | 8.0 | 8.0 | 2.0 |
| SORBITOL | 47.9 | 37.7 | 37.9 | 32.7 | 31.9 | 29.7 |
| MANNITOL | — | 10.0 | 10.0 | 10.0 | 10.0 | 6.0 |
| SORBITOL | 17.0 | 17.0 | — | — | — | — |
| LIQUID | | | | | | |
| LYCASIN | — | — | 17.0 | 12.0 | 8.0 | 20.0 |
| MALTITOL | — | — | — | 10.0 | — | — |
| XYLITOL | — | — | — | — | 15.0 | 15.0 |
| LACTITOL | — | — | — | — | — | — |
| FLAVOR | 1.5 | 1.5 | 1.5 | 1.5 | 1.5 | 1.5 |
| ACTIVE | 0.1 | 0.3 | 0.1 | 0.3 | 0.1 | 0.3 |
| WOLFBERRY | | | | | | |
| EXTRACT | | | | | | |
| | EX. 63 | EX. 64 | EX. 65 | EX. 66 | EX. 67 | EX. 68 |
| GUM BASE | 25.5 | 25.5 | 25.5 | 25.5 | 25.5 | 25.5 |
| GLYCERIN | 8.0 | 8.0 | 8.0 | 2.0 | 8.0 | 2.0 |
| SORBITOL | 41.9 | 36.7 | 31.9 | 40.7 | 29.9 | 29.7 |
| MANNITOL | 8.0 | 8.0 | 8.0 | — | — | — |
| SORBITOL | 5.0 | — | — | — | — | — |
| LIQUID | | | | | | |
| LYCASIN | — | 5.0 | 5.0 | 5.0 | 10.0 | 20.0 |
| MALTITOL | — | 5.0 | — | — | — | — |
| XYLITOL | — | — | — | 15.0 | 15.0 | 11.0 |
| LACTITOL | 10.0 | 10.0 | 10.0 | — | — | — |
| PALATINIT | — | — | 10.0 | 10.0 | 10.0 | 10.0 |
| FLAVOR | 1.5 | 1.5 | 1.5 | 1.5 | 1.5 | 1.5 |
| ACTIVE | 0.1 | 0.3 | 0.1 | 0.3 | 0.1 | 0.3 |
| WOLFBERRY | | | | | | |
| EXTRACT | | | | | | |

High-intensity sweeteners (HIS) such as aspartame, acesulfame K, or the salts of acesulfame, cyclamate and its salts, saccharin and its salts, alitame, neotame, sucralose, thaumatin, monellin, dihydrochalcone, stevioside, glycyrrhizin, and combinations thereof may be used in any of the Examples listed in Tables 3, 4, 5, 6, 7, 8, 9 and 10. Since Wolfberry extract may reduce sweetness, HIS may be used in sugar gum, and some of the alditols in sugar-free gum are less sweet than sugar so higher levels of HIS may be needed to obtain the proper level of sweetness.

High-intensity sweeteners may also be modified to control their release in those chewing gum formulations. This can be controlled by various methods of encapsulation, agglomeration, absorption, or a combination of methods to obtain either a fast or slow release of the sweetener. Sweetener combinations, some of which may be synergistic, may also be included in the gum formulations.

### Example 69

A 50% shellac, 50% active Wolfberry extract powder mixture is obtained by spray drying an appropriate ratio alcohol/shellac/Wolfberry extract mixture at 10% solids.

### Example 70

A 70% Zein, 30% active Wolfberry extract powder mixture is obtained by spray drying an alcohol/Zein/Wolfberry extract mixture at 10% solids.

### Example 71

A 40% shellac, 60% active Wolfberry extract powder mixture is obtained by fluid-bed coating Wolfberry extract with an alcohol/shellac solution at 20% solids.

### Example 72

A 40% Zein, 60% active Wolfberry extract powder mixture is obtained by fluid-bed coating Wolfberry extract with an alcohol/Zein solution of 20% solids.

### Example 73

A 70% wax, 30% active Wolfberry extract powder mixture is obtained by spray chilling a mixture of molten wax and Wolfberry extract.

### Example 74

A 70% Zein, 30% active Wolfberry extract powder mixture is obtained by spray drying an aqueous mixture of Wolfberry extract and Zein dispersed in an aqueous, high-pH (pH of 11.6-12.0) media at 10% solids.

Examples 69-74 would all give nearly complete encapsulation and would delay the release of Wolfberry extract when used in the sugarless gum formulation. The higher levels of coating would give a longer delayed release of sweetener than the lower levels of coating.

Other polymers that are more water soluble would have less of an effect of delaying the release of the Wolfberry extract if used in the coating.

### Example 75

A 30% hydroxypropylmethyl cellulose (HPMC), 70% Wolfberry extract powder mixture is obtained by fluid-bed coating Wolfberry extract with an aqueous solution of HPMC at 10% solids.

### Example 76

A 50% maltodextrin, 50% active Wolfberry extract powder mixture is obtained by spray drying an aqueous mixture of Wolfberry extract and maltodextrin at 20% solids.

### Example 77

A 40% gum arabic, 60% active Wolfberry extract powder mixture is obtained by fluid-bed coating Wolfberry extract with an aqueous solution of gum arabic at 20% solids.

The coated Wolfberry extract from Examples 75-77, when used in a chewing gum formula, would give a fast release of active agents.

Wolfberry extract could also be used in gum as an agglomerated active agent to give delayed sweetness release. Agglomerated active agent can be prepared as in the following examples:

### Example 78

A 15% hydroxypropylmethyl cellulose (HPMC), 85% active Wolfberry extract powder mixture is prepared by agglomerating Wolfberry extract and HPMC blended together, with water being added, and the resulting product being dried and ground.

### Example 79

A 15% gelatin, 85% active Wolfberry extract powder mixture is made by agglomerating Wolfberry extract and gelatin blended together, with water being added, and the resulting product being dried and ground.

As noted earlier, the gum formulas can be prepared as stick or tab products in the sugar or sugarless type formulations. These formulas can also be made in a pellet or pillow shape pellet or a round ball or any other shape of product for coating/panning. However, gum formulas are generally adjusted to a higher level of gum base to give a more consumer acceptable size of gum bolus.

Keeping this in mind, if a coating of about 25% of the total product is added to a pellet core as sugar or polyols, the gum base in the pellet core should also be increased by 25%. Likewise, if a 33% coating is applied, the base levels should also be increased by 33%. As a result, gum centers are usually formulated with about 25% to about 40% gum base with a corresponding decrease in the other ingredients except flavor. Generally flavors increase with the level of gum base as the base tends to bind flavors into the gum and more flavor is needed to give a good flavorful product. However flavors can also be added to the coating to give increased flavor impact and more flavor perception.

Some typical sugar type gum center formulations are shown in Table 11. Gum center formulas may or may not contain Wolfberry extract.

TABLE 11
(WEIGHT PERCENT)

| | EX. | EX. | | | | |
|---|---|---|---|---|---|---|
| | 80 | 81 | EX. 82 | EX. 83 | EX. 84 | EX. 85 |
| SUGAR | 52.0 | 48.7 | 47.55 | 44.0 | 40.7 | 38.55 |
| GUM BASE | 26.0 | 30.0 | 35.00 | 26.0 | 30.0 | 35.00 |
| CORN SYRUP | 20.0 | 19.0 | 15.00 | 18.0 | 17.0 | 14.00 |
| GLYCERIN | 1.0 | 1.0 | 1.00 | 1.0 | 1.0 | 1.00 |
| FLAVOR | 1.0 | 1.0 | 1.00 | 1.0 | 1.0 | 1.00 |
| COOLING AGENT | — | — | 0.05 | — | — | 0.05 |
| DEXTROSE | — | — | — | 10.0 | 10.0 | 10.00 |
| MONOHYDRATE | | | | | | |
| WOLFBERRY | — | 0.3 | 0.40 | — | 0.3 | 0.40 |
| EXTRACT | | | | | | |

Formulations with or without active Wolfberry extract can also be made similar to those found in Tables 3-8 for low, medium, and high moisture formulas. Higher levels of base may be used with a corresponding decrease in other ingredients. Also, other sugars and polyols may be used in the gum center as found in Tables 9-10. Wolfberry extract may be added to a gum center only, or to a gum coating with none in the center, or to both center and coating. Coated gum pieces are about 1.5 grams, so to obtain 3 mg of Wolfberry extract total piece must contain 0.2%.

Wolfberry extract can then be used in the coating formula on the various pellet gum formulations, as well as on various other confections such as hard candies, chewy candies, nougats, and the like. The following Table 12 shows some sugar and dextrose type formulas:

TABLE 12
(DRY WEIGHT PERCENT)

| | EX. 86 | EX. 87 | EX. 88 | EX. 89 | EX. 90 | EX. 91 |
|---|---|---|---|---|---|---|
| SUGAR | 97.1 | 95.2 | 93.5 | 96.9 | 94.9 | 93.0 |
| GUM ARABIC | 2.0 | 3.0 | 4.0 | 2.0 | 3.0 | 4.0 |
| TITANIUM | 0.5 | 1.0 | 1.0 | — | — | — |
| DIOXIDE | | | | | | |
| CALCIUM | — | — | — | 0.5 | 1.0 | 2.0 |
| CARBONATE | | | | | | |
| FLAVOR | 0.3 | 0.5 | 0.8 | 0.5 | 0.8 | 0.3 |
| WAX | 0.1 | 0.1 | 0.1 | 0.1 | 0.1 | 0.1 |
| WOLFBERRY | — | 0.2 | 0.6^{a)} | — | 0.2 | 0.6^{a)} |
| EXTRACT | | | | | | |

| | EX. 92 | EX. 93 | EX. 94 | EX. 95 |
|---|---|---|---|---|
| DEXTROSE | 97.6 | 95.2 | 97.0 | 93.9 |
| MONOHYDRATE | | | | |
| GUM ARABIC | 1.5 | 3.0 | 1.5 | 3.0 |
| TITANIUM | 0.5 | 1.0 | — | — |
| DIOXIDE | | | | |
| CALCIUM | — | — | 1.0 | 2.0 |
| CARBONATE | | | | |
| FLAVOR | 0.3 | 0.5 | 0.2 | 0.4 |
| WAX | 0.1 | 0.1 | 0.1 | 0.1 |
| WOLFBERRY | — | 0.2 | 0.2 | 0.6^{a)} |
| EXTRACT | | | | |

| | EX. | EX. | | | | |
|---|---|---|---|---|---|---|
| | 96 | 97 | EX. 98 | EX. 99 | EX. 100 | EX. 101 |
| SUGAR | 77.5 | 81.2 | — | — | 86.9 | — |
| DEXTROSE | — | — | 77.5 | 86.1 | — | 86.5 |
| MONOHYDRATE | | | | | | |
| POWDER SUGAR | 20.0 | 15.0 | — | — | — | — |
| POWDER | — | — | 20.0 | 10.0 | — | — |
| DEXTROSE | | | | | | |
| GUM ARABIC | 2.0 | 3.0 | 2.0 | 3.0 | 8.0 | 8.0 |
| POWDER | | | | | | |
| GUM ARABIC | — | — | — | — | 4.0 | 4.0 |
| SOLUTION | | | | | | |
| FLAVOR | 0.4 | 0.5 | 0.4 | 0.6 | 0.4 | 0.8 |
| WAX | 0.1 | 0.1 | 0.1 | 0.1 | 0.1 | 0.1 |
| WOLFBERRY | — | 0.2 | — | 0.2 | 0.6^{a} | 0.6^{a} |
| EXTRACT | | | | | | |

^{a)}All of the active agent is in the coating, which comprises 33% of the product.

The above process gives a hard shell coating. Often a dry charge of powdered sugar or dextrose monohydrate may be used. This gives a somewhat softer coating. A dry charge may be used to build up a coating, but then finished with a straight syrup to obtain a hard shell. Table 12 gives these types of formulas.

In Examples 96-99, gum arabic is blended in the sugar syrup. In Examples 100 and 101, gum arabic powder is dry charged after a gum arabic solution is applied in the first stages of coating, then this is followed by a hard shell coating of sugar solution or dextrose solution.

The above formulations are made by making a syrup by dissolving the sugar and gum arabic in solution at about 75% solids at boiling, and suspending titanium dioxide or calcium carbonate in this syrup. Some of the dextrose may be added as a dry charge which may also contain the active agent. Wolfberry extract may be dissolved in water, not mixed with hot syrup, but added between coatings, or it may be added to the hot syrup and used in the early stages of coating or used throughout the coating process. Flavor is not mixed with the hot syrup, but added at low levels with one or more coats. Wolfberry extract may be dissolved in flavor and added to the coating. After the final coats are applied and dried, wax is applied to give a smooth polish.

Wolfberry extract may also be used in coating of sugarless gum centers. Like sugar gum centers, the base formulation can be increased in proportion to the amount of coating applied to the center. Formulations with and without Wolfberry extract similar to those found in Tables 6, 7 or 8 for low and high moisture gum can be used to make gum centers. Generally, the base level may be increased to 30-46% with the other ingredients proportionally reduced. Some typical gum formulas are in Table 13.

TABLE 13
(WEIGHT PERCENT)

| | EX. 102 | EX. 103 | EX. 104^{c)} | EX. 105 | EX. 106 | EX. 107 | EX. 108^{c)} |
|---|---|---|---|---|---|---|---|
| GUM BASE | 35.0 | 35.0 | 30.0 | 30.0 | 30.0 | 40.0 | 50.0 |
| CALCIUM | — | — | 5.0 | 10.0 | 15.0 | — | — |
| CARBONATE | | | | | | | |
| SORBITOL | 43.3 | 45.0 | 45.9 | 40.3 | 44.5 | 41.4 | 26.1 |
| MANNITOL | 10.0 | 10.0 | 5.0 | 10.0 | — | 8.0 | 10.0 |
| GLYCERIN | — | 8.0 | 2.0 | — | 8.0 | 2.0 | 2.0 |
| SORBITOL | 10.0 | — | 10.0 | 8.0 | — | 6.0^{a)} | 10.0^{a)} |
| LIQUID | | | | | | | |
| FLAVOR | 1.5 | 1.5 | 1.5 | 1.5 | 2.0 | 2.0 | 1.3 |
| HIGH-INTENSITY | 0.2 | 0.2 | 0.2 | 0.2 | 0.2 | 0.3 | 0.2 |
| SWEETENER | | | | | | | |
| WOLFBERRY | — | 0.3 | 0.4 | — | 0.3 | 0.3 | 0.4 |
| EXTRACT^{b)} | | | | | | | |

^{a)}Lycasin brand hydrogenated starch hydrolyzate used instead of sorbitol liquid
^{b)}This material may be dissolved in water, glycerin, sorbitol liquid, or HSH.
^{c)}These formulas require 50% of the product to be a coating with no active agent, to give a final product with 0.2% active agent.

In the above center formulations, the high intensity sweetener used is aspartame. However other high intensity such as alitame, acesulfame K, salts of acesulfame, cyclamate and its salts, saccharin and its salts, neotame, sucralose, thaumatin, monellin, dihydrochalcone, stevioside, glycyrrhizin and combinations thereof may be used in any of the examples with the level adjusted for sweetness.

Lycasin and other polyols such as maltitol, xylitol, lactitol and hydrogenated isomaltulose may also be used in the gum center formulations at various levels similar to those shown in Table 10. The texture may be adjusted by varying glycerin or sorbitol liquid. Sweetness of the center formulation can also be adjusted by varying the level of high intensity sweetener.

Wolfberry extract may be used in sugarless coatings with xylitol, sorbitol, maltitol, lactitol, hydrogenated isomaltulose and erythritol. The following table gives formulas for a xylitol coating:

TABLE 14
(DRY WEIGHT PERCENT)

| | EX. 109 | EX. 110 | EX. 111 | EX. 112 | EX. 113 | EX. 114 |
|---|---|---|---|---|---|---|
| XYLITOL | 94.8 | 92.2 | 90.1 | 90.1 | 89.7 | 88.2 |
| GUM ARABIC | 4.0 | 6.0 | 7.0 | 8.5 | 8.5 | 10.0 |
| FLAVOR | 0.5 | 0.5 | 0.7 | 0.7 | 0.9 | 0.5 |
| TITANIUM | 0.5 | 0.9 | — | 0.5 | 0.5** | 0.5** |
| DIOXIDE | | | | | | |
| TALC | 0.1 | 0.1 | 0.1 | 0.1 | 0.1 | 0.1 |
| WAX | 0.1 | 0.1 | 0.1 | 0.1 | 0.1 | 0.1 |
| COLOR* | — | — | 1.4 | — | — | — |
| WOLFBERRY | — | 0.2 | 0.6^{a)} | — | 0.2 | 0.6^{a)} |
| EXTRACT | | | | | | |

*Lake color dispersed in xylitol solution
**Calcium carbonate used in place of titanium dioxide
^{a)}All of the active agent is in the gum coating, which comprises 33% of the gum product.

The above formulas are used to coat pellets by applying a xylitol/gum arabic solution in multiple coats and air drying. Color or whitener is also mixed in the solution. Wolfberry extract may be dissolved in water and added between coating applications or mixed with the hot syrup and used in the early stages of coating or used throughout the coating process. After pellets have been coated and dried, talc and wax are added to give a polish.

For examples 115-120, erythritol may be substituted for xylitol in Table 14. In some cases more gum arabic may be needed to give good binding.

For coating formulas based on sorbitol, maltitol, lactitol, and hydrogenated isomaltulose, gum arabic can be used as a binder and film former, and a crystallization modifier to help facilitate coating. Generally these polyols are more difficult to coat using only a straight syrup, but with proper technique a good smooth hard shell can be made. However, it may be preferable to add a dry charge to quicken the drying process before the pellets get too sticky. The following formulations may be used.

TABLE 15
(DRY WEIGHT PERCENT)

| | EX. 121 | EX. 122 | EX. 123 | EX. 124 | EX. 125 | EX. 126 |
|---|---|---|---|---|---|---|
| MALTITOL | 96.8 | 94.7 | 91.5 | 86.8 | 75.9 | 68.9 |
| MALTITOL | — | — | — | 10.0 | 20.0 | 25.0 |
| POWDER | | | | | | |
| GUM ARABIC | 2.0 | 4.0 | 6.0 | 2.0 | 3.0 | 4.0 |
| FLAVOR | 0.5 | 0.4 | 0.7 | 0.5 | 0.3 | 0.7 |
| TITANIUM | 0.5 | 0.5 | 1.0 | 0.5 | 0.4 | 0.6 |
| DIOXIDE | | | | | | |
| TALC | 0.1 | 0.1 | 0.1 | 0.1 | 0.1 | 0.1 |
| WAX | 0.1 | 0.1 | 0.1 | 0.1 | 0.1 | 0.1 |
| WOLFBERRY | — | 0.2 | 0.6^{a)} | — | 0.2 | 0.6^{a)} |
| EXTRACT | | | | | | |

^{a)}All of the active agent is in the coating, which comprises 33% of the product.

Maltitol powder is used to dry charge in the early stages of coating. Maltitol, gum arabic, and whitener are blended into a syrup and applied to pellets. Wolfberry extract may be applied in a similar manner as in the previous xylitol coating or may be preblended with the dry charge material. After all coating is applied and dried, talc and wax are added to give a polish.

In a similar manner, coatings with sorbitol, lactitol, and hydrogenated isomaltulose may be made in the coating formulas in Table 15 by replacing maltitol with any one of the other polyols and maltitol powder with the polyol powder. Like maltitol, the other polyols may become sticky during the coating and drying process, so the dry powder charge may be needed to give the proper drying. In the later stages of the coating process, less gum arabic could be used and a more pure polyol syrup could be used to give a smooth surface. Also, the dry charge would only be used in the early stages of the coating process.

In addition to dry charging with the specific polyol, other ingredients may be added to the dry charge to help absorb moisture. These materials could be inert such as talc, calcium carbonate, magnesium carbonate, starches, gums like Wolfberry extract, gum talha, gum arabic or other moisture absorbing materials. Also, powdered sweeteners or flavors could be added with the dry charge, along with the active medicament.

Some polyols such as sorbitol, maltitol, lactitol, erythritol, or hydrogenated isomaltulose are not sufficiently sweet compared to sugar or xylitol, so high intensity sweeteners may be added to the coating, such as aspartame, acesulfame K, salts of acesulfame, cyclamate and its salts, saccharin and its salts, alitame, sucralose, thaumatin, monellin, dihydrochalcone, glycyrrhizin, neotame, and combinations thereof. If a hot syrup is applied, heat may degrade the sweetener so only stable sweeteners should be used. Generally high intensity sweeteners are added with the polyol/gum arabic solution to obtain an even distribution in the coatings.

The formulas listed in Table 16 comprise various sugar-type formulas in which Chinese hawthorn extract can be added to gum after it is dissolved in water or mixed with various aqueous solvents. Chinese hawthorn extract is an active medicament used as an antihistamine. These formulas give a 3 gram stick with 4 mg of Chinese hawthorn extract.

TABLE 16
(WEIGHT PERCENT)

| | EX. 127 | EX. 128 | EX. 129 | EX. 130 | EX. 131 | EX. 132 | EX. 133 | EX. 134 |
|---|---|---|---|---|---|---|---|---|
| SUGAR | 62.47 | 64.3 | 63.0 | 64.4 | 64.4 | 62.7 | 61.6 | 47.0 |
| BASE | 19.2 | 19.2 | 19.2 | 19.2 | 19.2 | 19.2 | 19.2 | 19.2 |
| CORN SYRUP | 15.9 | 12.9 | 12.9 | 12.9 | 12.9 | 15.9 | 0.0 | 2.9 |
| PEPPER-MINT | 0.9 | 0.9 | 0.9 | 0.9 | 0.0 | 0.0 | 0.9 | 0.9 |
| FLAVOR | | | | | | | | |
| GLYCERIN | 1.4 | 1.4 | 1.4 | 0.0 | 2.2 | 0.9 | 1.4 | 0.0 |
| LIQUID/ACTIVE | 0.13 | 1.3 | 2.6 | 2.6 | 1.3 | 1.3 | 16.9 | 30.0 |
| BLEND | | | | | | | | |

### Example 127

Chinese hawthorn extract powder can be added directly to the gum.

### Example 128

A 1 gram quantity of Chinese hawthorn extract can be dissolved in 9 grams of water giving a 10% solution and added to gum.

### Example 129

A 1 gram quantity of Chinese hawthorn extract can be dissolved in 9 grams of water and mixed with 10 grams of glycerin and added to the gum.

### Example 130

A 1 gram quantity of Chinese hawthorn extract is mixed with 19 grams of glycerin giving a 5% solution and added to gum.

### Example 131

A 1 gram quantity of Chinese hawthorn extract is mixed with 9 grams of peppermint flavor giving a 10% solution and added to gum.

### Example 132

A 1 gram quantity of Chinese hawthorn extract is dissolved in 9 grams of ethanol, which is then mixed with 90 grams of peppermint flavor and added to gum.

### Example 133

A 1.3 gram quantity of Chinese hawthorn extract is dissolved in 168 grams of corn syrup and added to chewing gum.

### Example 134

To a 200 gram quantity of corn syrup is added 100 grams of glycerin. To this mixture is added 1.3 gram of Chinese hawthorn extract and blended. This mixture is then added to gum.

In the next examples of sugar formulations, Chinese hawthorn extract can be dissolved in water and emulsifiers can be added to the aqueous solution. Example solutions can be prepared by dissolving 13 grams of Chinese hawthorn extract in 72 grams of water and adding 15 grams of emulsifiers of various hydrophilic-lipophilic balance (HLB) values to the solution. The mixtures can then be used in the following formulas. Example 135 uses a mixture of Chinese hawthorn extract and water with no emulsifier. The HLB value of the emulsifiers used in Examples 136-140 are listed in Table 17.

TABLE 17
(WEIGHT PERCENT)

| | EX. 135 | EX. 136 | EX. 137 | EX. 138 | EX. 139 | EX. 140 |
|---|---|---|---|---|---|---|
| SUGAR | 54.7 | 54.7 | 54.7 | 54.7 | 54.7 | 54.7 |
| BASE | 19.2 | 19.2 | 19.2 | 19.2 | 19.2 | 19.2 |
| CORN SYRUP | 12.9 | 12.9 | 12.9 | 12.9 | 12.9 | 12.9 |
| GLYCERIN | 1.4 | 1.4 | 1.4 | 1.4 | 1.4 | 1.4 |
| DEXTROSE | 9.9 | 9.9 | 9.9 | 9.9 | 9.9 | 9.9 |
| MONOHYDRATE | | | | | | |
| COOLING AGENT | — | — | 0.1 | 0.2 | — | — |
| FLAVOR | 0.9 | 0.9 | 0.8 | 0.7 | 0.9 | 0.9 |
| ACTIVE AGENT | 1.0 | 1.0 | 1.0 | 1.0 | 1.0 | 1.0 |
| EMULSIFIER/ | | | | | | |
| WATER MIXTURE | | | | | | |
| | None | HLB = 2 | HLB = 4 | HLB = 6 | HLB = 9 | HLB = 12 |

### Examples 141-146

The same as the formulations made in Examples 135-140, respectively, except that the flavor can be mixed together with the aqueous active agent solution and emulsified before adding the mixture to the gum batch.

The following Tables 18 through 25 are examples of gum formulations that demonstrate formula variations in which Chinese hawthorn extract may be used. The active agent may be added with or without encapsulation, or may be treated for fast release.

Examples 147-150 in Table 18 demonstrate the use of Chinese hawthorn extract in low-moisture sugar formulations showing less than 2% theoretical moisture:

TABLE 18
(WEIGHT PERCENT)

| | EX. 147 | EX. 148 | EX. 149 | EX. 150 |
|---|---|---|---|---|
| SUGAR | 58.77 | 58.51 | 58.77 | 54.51 |
| GUM BASE | 19.2 | 19.2 | 19.2 | 19.2 |
| CORN^{a} SYRUP | 6.0 | 6.0 | — | — |
| DEXTROSE MONOHYDRATE | 10.0 | 10.0 | 10.0 | 10.0 |
| LACTOSE | 0.0 | 0.0 | 0.0 | 5.0 |
| GLYCERIN^{b} | 5.0 | 5.0 | 11.0 | 10.0 |
| FLAVOR | 0.9 | 0.9 | 0.9 | 0.9 |
| CHINESE HAWTHORN | 0.13 | 0.39 | 0.13 | 0.39 |
| EXTRACT | | | | |

^{a}Corn syrup is evaporated to 85% solids, 15% moisture
^{b}Glycerin and syrup may be blended and co-evaporated

Examples 151-154 in Table 19 demonstrate the use of Chinese hawthorn extract in medium-moisture sugar formulations having about 2% to about 5% moisture.

Examples 155-158 in Table 20 demonstrate the use of Chinese hawthorn extract in high-moisture sugar formulations having more than about 5% moisture.

TABLE 19
(WEIGHT PERCENT)

| | EX. 151 | EX. 152 | EX. 153 | EX. 154 |
|---|---|---|---|---|
| SUGAR | 53.37 | 53.11 | 53.37 | 49.61 |
| GUM BASE | 19.2 | 19.2 | 19.2 | 19.2 |
| CORN SYRUP^{a)} | 15.0 | 15.0 | 13.0 | 12.5 |
| DEXTROSE MONOHYDRATE | 10.0 | 10.0 | 10.0 | 10.0 |
| GLYCERIN^{b)} | 1.4 | 1.4 | 3.4 | 7.4 |
| FLAVOR | 0.9 | 0.9 | 0.9 | 0.9 |
| CHINESE HAWTHORN | 0.13 | 0.39 | 0.13 | 0.39 |
| EXTRACT | | | | |

^{a)}Corn syrup is evaporated to 85% solids, 15% moisture
^{b)}Glycerin and syrup may be blended and co-evaporated

TABLE 20
(WEIGHT PERCENT)

| | EX. 155 | EX. 156 | EX. 157 | EX. 158 |
|---|---|---|---|---|
| SUGAR | 50.87 | 50.61 | 49.87 | 49.61 |
| GUM BASE | 24.0 | 24.0 | 24.0 | 24.0 |
| CORN SYRUP | 24.0 | 24.0 | 24.0 | 24.6 |
| GLYCERIN | 0.0 | 0.0 | 1.0 | 0.4 |
| FLAVOR | 1.0 | 1.0 | 1.0 | 1.0 |
| CHINESE HAWTHORN | 0.13 | 0.39 | 0.13 | 0.39 |
| EXTRACT | | | | |

Examples 159-162 in Table 21 and Examples 163-170 in Tables 22 and 23 demonstrate the use of Chinese hawthorn extract in low- and high-moisture gums that are sugar-free. Low-moisture gums have less than about 2% moisture, and high-moisture gums have greater than 2% moisture.

TABLE 21
(WEIGHT PERCENT)

| | EX. 159 | EX. 160 | EX. 161 | EX. 162 |
|---|---|---|---|---|
| BASE | 25.5 | 25.5 | 25.5 | 25.5 |
| SORBITOL | 50.87 | 50.61 | 48.87 | 45.61 |
| MANNITOL | 12.0 | 12.0 | 12.0 | 12.0 |
| GLYCERIN | 10.0 | 10.0 | 12.0 | 15.0 |
| COOLING AGENT | 0.2 | 0.1 | — | — |
| FLAVOR | 1.3 | 1.4 | 1.5 | 1.5 |
| CHINESE HAWTHORN | 0.13 | 0.39 | 0.13 | 0.39 |
| EXTRACT | | | | |

TABLE 22
(WEIGHT PERCENT)

| | EX. 163 | EX. 164 | EX. 165 | EX. 166 |
|---|---|---|---|---|
| BASE | 25.5 | 25.5 | 25.5 | 25.5 |
| SORBITOL | 50.87 | 50.61 | 40.87 | 40.61 |
| LIQUID SORBITOL* | 10.0 | 10.0 | 20.0 | 20.0 |
| MANNITOL | 10.0 | 10.0 | 10.0 | 10.0 |
| GLYCERIN | 2.0 | 2.0 | 2.0 | 2.0 |
| FLAVOR | 1.5 | 1.5 | 1.5 | 1.5 |
| CHINESE HAWTHORN | 0.13 | 0.39 | 0.13 | 0.39 |
| EXTRACT | | | | |

*Sorbitol liquid contains 70% sorbitol, 30% water

TABLE 23
(WEIGHT PERCENT)

| | EX. 167 | EX. 168 | EX. 169 | EX. 170 |
|---|---|---|---|---|
| BASE | 25.5 | 25.5 | 25.5 | 25.5 |
| SORBITOL | 50.87 | 48.61 | 44.87 | 42.61 |
| HSH SYRUP* | 10.0 | 10.0 | 10.0 | 10.0 |
| MANNITOL | 8.0 | 8.0 | 8.0 | 8.0 |
| GLYCERIN** | 4.0 | 6.0 | 10.0 | 12.0 |
| COOLING AGENT | 0.2 | — | 0.2 | 0.1 |
| FLAVOR | 1.3 | 1.5 | 1.3 | 1.4 |
| CHINESE HAWTHORN | 0.13 | 0.39 | 0.13 | 0.39 |
| EXTRACT | | | | |

*Hydrogenated starch hydrolyzate syrup
**Glycerin and HSH syrup may be blended or co-evaporated

Table 24 shows sugar chewing formulations that can be made with various types of sugars.

TABLE 24
(WEIGHT PERCENT)

| | EX. 171 | EX. 172 | EX. 173 | EX. 174 | EX. 175 | EX. 176 |
|---|---|---|---|---|---|---|
| GUM BASE | 19.2 | 19.2 | 19.2 | 19.2 | 19.2 | 19.2 |
| SUCROSE | 49.37 | 48.11 | 44.37 | 39.11 | 34.37 | 42.11 |
| GLYCERIN | 1.4 | 2.4 | 1.4 | 6.4 | 1.4 | 3.4 |
| CORN SYRUP | 14.0 | 14.0 | 14.0 | 14.0 | 14.0 | 14.0 |
| DEXTROSE | 5.0 | 5.0 | — | — | 10.0 | 5.0 |
| LACTOSE | 5.0 | 5.0 | 10.0 | 10.0 | — | — |
| FRUCTOSE | 5.0 | 5.0 | 10.0 | 10.0 | 10.0 | 5.0 |
| INVERT | — | — | — | — | 10.0 | 10.0 |
| SUGAR | | | | | | |
| MALTOSE | — | — | — | — | — | — |
| CORN SYRUP | — | — | — | — | — | — |
| SOLIDS | | | | | | |
| PEPPERMINT | 0.9 | 0.9 | 0.9 | 0.9 | 0.9 | 0.9 |
| FLAVOR | | | | | | |
| CHINESE | 0.13 | 0.39 | 0.13 | 0.39 | 0.13 | 0.39 |
| HAWTHORN | | | | | | |
| EXTRACT | | | | | | |
| | EX. 177 | EX. 178 | EX. 179 | EX. 180 | EX. 181 | EX. 182 |
| GUM BASE | 19.2 | 19.2 | 19.2 | 19.2 | 19.2 | 19.2 |
| SUCROSE | 34.37 | 43.11 | 34.37 | 43.11 | 42.37 | 45.06 |
| GLYCERIN | 1.4 | 2.4 | 1.4 | 2.4 | 1.4 | 3.4 |
| CORN SYRUP | 14.0 | 14.0 | 14.0 | 14.0 | 11.0 | 11.0 |
| DEXTROSE | 10.0 | 5.0 | 10.0 | 5.0 | 10.0 | 5.0 |
| LACTOSE | — | — | — | — | — | — |
| FRUCTOSE | 10.0 | 5.0 | 10.0 | 5.0 | 5.0 | 5.0 |
| INVERT | 10.0 | 10.0 | — | — | 5.0 | 5.0 |
| SUGAR | | | | | | |
| MALTOSE | — | — | 10.0 | 10.0 | — | — |
| CORN SYRUP | — | — | — | — | 5.0 | 5.0 |
| SOLIDS | | | | | | |
| COOLING | 0.1 | — | — | 0.3 | — | 0.05 |
| AGENT | | | | | | |
| PEPPERMINT | 0.8 | 0.9 | 0.9 | 0.6 | 0.9 | 0.9 |
| FLAVOR | | | | | | |
| CHINESE | 0.13 | 0.39 | 0.13 | 0.39 | 0.13 | 0.39 |
| HAWTHORN | | | | | | |
| EXTRACT | | | | | | |

Table 25 shows chewing gum formulations that are free of sugar. These formulations can use a wide variety of other non-sugar alditols.

TABLE 25
(WEIGHT PERCENT)

| | EX. 183 | EX. 184 | EX. 185 | EX. 186 | EX. 187 | EX. 188 |
|---|---|---|---|---|---|---|
| GUM BASE | 25.5 | 25.5 | 25.5 | 25.5 | 25.5 | 25.5 |
| GLYCERIN | 8.0 | 8.0 | 8.0 | 8.0 | 8.0 | 2.0 |
| SORBITOL | 47.87 | 37.611 | 37.87 | 32.61 | 31.87 | 29.61 |
| MANNITOL | — | 10.0 | 10.0 | 10.0 | 10.0 | 6.0 |
| SORBITOL | 17.0 | 17.0 | — | — | — | — |
| LIQUID | | | | | | |
| LYCASIN | — | — | 17.0 | 12.0 | 8.0 | 20.0 |
| MALTITOL | — | — | — | 10.0 | — | — |
| XYLITOL | — | — | — | — | 15.0 | 15.0 |
| FLAVOR | 1.5 | 1.5 | 1.5 | 1.5 | 1.5 | 1.5 |
| CHINESE | 0.13 | 0.39 | 0.13 | 0.39 | 0.13 | 0.39 |
| HAWTHORN | | | | | | |
| EXTRACT | | | | | | |
| | EX. 189 | EX. 190 | EX. 191 | EX. 192 | EX. 193 | EX. 194 |
| GUM BASE | 25.5 | 25.5 | 25.5 | 25.5 | 25.5 | 25.5 |
| GLYCERIN | 8.0 | 8.0 | 8.0 | 2.0 | 8.0 | 2.0 |
| SORBITOL | 41.87 | 36.61 | 31.87 | 40.61 | 29.87 | 29.61 |
| MANNITOL | 8.0 | 8.0 | 8.0 | — | — | — |
| SORBITOL | 5.0 | — | — | — | — | — |
| LIQUID | | | | | | |
| LYCASIN | — | 5.0 | 5.0 | 5.0 | 10.0 | 20.0 |
| MALTITOL | — | 5.0 | — | — | — | — |
| XYLITOL | — | — | — | 15.0 | 15.0 | 11.0 |
| LACTITOL | 10.0 | 10.0 | 10.0 | — | — | — |
| PALATINIT | — | — | 10.0 | 10.0 | 10.0 | 10.0 |
| COOLING | 0.2 | — | 0.1 | — | — | — |
| AGENT | | | | | | |
| FLAVOR | 1.3 | 1.5 | 1.4 | 1.5 | 1.5 | 1.5 |
| CHINESE | 0.13 | 0.39 | 0.13 | 0.39 | 0.13 | 0.39 |
| HAWTHORN | | | | | | |
| EXTRACT | | | | | | |

High-intensity sweeteners (HIS) such as aspartame, acesulfame K, or the salts of acesulfame, cyclamate and its salts, saccharin and its salts, alitame, neotame, sucralose, thaumatin, monellin, dihydrochalcone, stevioside, glycyrrhizin, and combinations thereof may be used in any of the Examples listed in Tables 18-25. Since Chinese hawthorn extract may reduce sweetness, HIS may be used in sugar gum, and some of the alditols in sugar-free gum are less sweet than sugar so higher levels of HIS may be needed to obtain the proper level of sweetness.

High-intensity sweeteners (HIS) may also be modified to control their release in those chewing gum formulations. This can be controlled by various methods of encapsulation, agglomeration, absorption, or a combination of methods to obtain either a fast or slow release of the sweetener. Sweetener combinations, some of which may be synergistic, may also be included in the gum formulations.

As noted earlier, the gum formulas can be prepared as stick or tab products in the sugar or sugarless type formulations. These formulas can also be made in a pellet or pillow shape pellet or a round ball or any other shape of product for coating/panning. However, gum formulas are generally adjusted to a higher level of gum base to give a more consumer acceptable size of gum bolus.

As a result, gum centers are usually formulated with about 25% to about 40% gum base with a corresponding decrease in the other ingredients except flavor. Generally flavors increase with the level of gum base as the base tends to bind flavors into the gum and more flavor is needed to give a good flavorful product. However flavors can also be added to the coating to give increased flavor impact and more flavor perception.

Some typical sugar type gum center formulations are shown in Table 26. Gum center formulas may or may not contain Chinese hawthorn extract.

TABLE 26
(WEIGHT PERCENT)

| | | EX. | EX. | | | |
|---|---|---|---|---|---|---|
| | EX. 195 | 196 | 197 | EX. 198 | EX. 199 | EX. 200 |
| SUGAR | 52.0 | 48.73 | 47.59 | 44.0 | 40.73 | 38.59 |
| GUM BASE | 26.0 | 30.0 | 35.00 | 26.0 | 30.0 | 35.00 |
| CORN SYRUP | 20.0 | 19.0 | 15.00 | 18.0 | 17.0 | 14.00 |
| GLYCERIN | 1.0 | 1.0 | 1.00 | 1.0 | 1.0 | 1.00 |
| PEPPERMINT | 1.0 | 1.0 | 1.00 | 1.0 | 1.0 | 1.00 |
| FLAVOR | | | | | | |
| DEXTROSE | — | — | — | 10.0 | 10.0 | 10.00 |
| MONOHYDRATE | | | | | | |
| CHINESE | —^{a)} | 0.27 | 0.41 | —^{a)} | 0.27 | 0.41 |
| HAWTHORN | | | | | | |
| EXTRACT | | | | | | |

^{a)}All of the active agent is in the coating, which comprises 33% of the product.

Formulations with or without Chinese hawthorn extract can also be made similar to those found in Tables 18-23 for low, medium, and high moisture formulas. Higher levels of base may be used with a corresponding decrease in other ingredients. Also, other sugars are polyols may be used in the gum center as found in Tables 24 and 25. Chinese hawthorn extract may be added to a gum center only, or to a gum coating with none in the center, or to both center and coating. Coated gum pieces are about 1.5 grams, so to obtain 4 mg of Chinese hawthorn extract total piece must contain 0.27%.

Chinese hawthorn extract can be used in the coating formula on the various pellet gum formulations. The following Table 27 shows some sugar and dextrose type formulas:

TABLE 27
(DRY WEIGHT PERCENT)

| | EX. 201 | EX. 202 | EX. 203 | EX. 204 | EX. 205 | EX. 206 |
|---|---|---|---|---|---|---|
| SUGAR | 97.1 | 95.13 | 93.29 | 96.9 | 94.83 | 92.79 |
| GUM ARABIC | 2.0 | 3.0 | 4.0 | 2.0 | 3.0 | 4.0 |
| TITANIUM DIOXIDE | 0.5 | 1.0 | 1.0 | — | — | — |
| CALCIUM CARBONATE | — | — | — | 0.5 | 1.0 | 2.0 |
| FLAVOR | 0.3 | 0.5 | 0.8 | 0.5 | 0.8 | 0.3 |
| WAX | 0.1 | 0.1 | 0.1 | 0.1 | 0.1 | 0.1 |
| CHINESE HAWTHORN | — | 0.27 | 0.81^{a)} | — | 0.27 | 0.81^{a)} |
| EXTRACT | | | | | | |

| | EX. 207 | EX. 208 | EX. 209 | EX. 210 |
|---|---|---|---|---|
| DEXTROSE MONOHYDRATE | 97.6 | 95.13 | 96.90 | 93.69 |
| GUM ARABIC | 1.5 | 3.0 | 1.5 | 3.0 |
| TITANIUM DIOXIDE | 0.5 | 1.0 | — | — |
| COOLING AGENT | — | 0.1 | 0.03 | |
| CALCIUM CARBONATE | — | — | 1.0 | 2.0 |
| FLAVOR | 0.3 | 0.4 | 0.2 | 0.4 |
| WAX | 0.1 | 0.1 | 0.1 | 0.1 |
| CHINESE HAWTHORN | — | 0.27 | 0.27 | 0.81^{a)} |
| EXTRACT | | | | |

^{a)}All of the active agent is in the coating, which comprises 33% of the product.

The above formulations are made by making a syrup by dissolving the sugar and gum arabic in solution at about 75% solids at boiling, and suspending titanium dioxide or calcium carbonate in this syrup. Some of the dextrose may be added as a dry charge, which may also contain the active. Chinese hawthorn extract may be dissolved in water, not mixed with hot syrup, but applied between coatings, or it may be added to the hot syrup and used in the early stages of coating or used throughout the coating process. Flavor is not mixed with the hot syrup, but added at low levels with one or more coats. Chinese hawthorn extract may be dissolved in flavor and added to the coating. After the final coats are applied and dried, wax is applied to give a smooth polish.

Chinese hawthorn extract may also be used in coating of sugarless gum centers. Like sugar gum centers, the base formulation can be increased in proportion to the amount of coating applied to the center. Formulations with and without Chinese hawthorn extract similar to those found in Tables 21-25 for low and high moisture gum can be used to make gum centers. Generally, the base level may be increased to 30-46% with the other ingredients proportionally reduced. Some typical gum formulas are in Table 28.

TABLE 28
(WEIGHT PERCENT)

| | EX. 211 | EX. 212 | EX. 213 | EX. 214 | EX. 215 | EX. 216 | EX. 217 |
|---|---|---|---|---|---|---|---|
| GUM BASE | 35.0 | 35.0 | 30.0 | 30.0 | 30.0 | 40.0 | 50.0 |
| CALCIUM CARBONATE | — | — | 5.0 | 10.0 | 15.0 | — | — |
| SORBITOL | 43.3 | 45.03 | 45.89 | 40.3 | 44.53 | 41.29 | 25.96 |
| MANNITOL | 10.0 | 10.0 | 5.0 | 10.0 | — | 8.0 | 10.0 |
| GLYCERIN | — | 8.0 | 2.0 | — | 8.0 | 2.0 | 2.0 |
| SORBITOL LIQUID | 10.0 | — | 10.0 | 8.0 | — | 6.0^{a)} | 10.0^{a)} |
| FLAVOR | 1.5 | 1.5 | 1.5 | 1.5 | 2.0 | 2.0 | 1.3 |
| HIGH INTENSITY | 0.2 | 0.2 | 0.2 | 0.2 | 0.2 | 0.3 | 0.2 |
| SWEETENER | | | | | | | |
| CHINESE HAWTHORN | —^{c)} | 0.27 | 0.41 | —^{c)} | 0.27 | 0.41 | 0.54^{d)} |
| EXTRACT^{b)} | | | | | | | |

^{a)}Lycasin brand hydrogenated starch hydrolyzate used instead of sorbitol liquid
^{b)}This material may be dissolved in water, glycerin, sorbitol liquid, or HSH.
^{c)}All of the active agent is in the coating, which comprises 33% of the product.
^{d)}This example required 50% of the product to be a coating with no active agent in the coating, to give a gum product with 0.27% active agent.

In the above center formulations, the high intensity sweetener used is aspartame. However other high intensity such as alitame, acesulfame K, salts of acesulfame, cyclamate and its salts, saccharin and its salts, neotame, sucralose, thaumatin, monellin, dihydrochalcone, stevioside, glycyrrhizin and combinations thereof may be used in any of the examples with the level adjusted for sweetness.

Lycasin and other polyols such as maltitol, xylitol, lactitol and hydrogenated isomaltulose may also be used in the gum center formulations at various levels similar to those shown in Table 25. The texture may be adjusted by varying glycerin or sorbitol liquid. Sweetness of the center formulation can also be adjusted by varying the level of high intensity sweetener.

Chinese hawthorn extract may be used in sugarless coatings with xylitol, sorbitol, maltitol, lactitol, hydrogenated isomaltulose and erythritol. The following table gives formulas for a xylitol coating:

TABLE 29
(DRY WEIGHT PERCENT)

| | EX. 218 | EX. 219 | EX. 220 | EX. 221 | EX. 222 | EX. 223 |
|---|---|---|---|---|---|---|
| XYLITOL | 94.8 | 92.13 | 89.89 | 90.1 | 89.63 | 87.99 |
| GUM ARABIC | 4.0 | 6.0 | 7.0 | 8.5 | 8.5 | 10.0 |
| FLAVOR | 0.4 | 0.5 | 0.7 | 0.6 | 0.9 | 0.5 |
| COOLING AGENT | 0.1 | — | — | 0.1 | — | — |
| TITANIUM DIOXIDE | 0.5 | 0.9 | — | 0.5 | 0.5** | 0.5** |
| TALC | 0.1 | 0.1 | 0.1 | 0.1 | 0.1 | 0.1 |
| WAX | 0.1 | 0.1 | 0.1 | 0.1 | 0.1 | 0.1 |
| COLOR* | — | — | 1.4 | — | — | — |
| CHINESE | — | 0.27 | 0.81^{a)} | — | 0.27 | 0.81^{a)} |
| HAWTHORN | | | | | | |
| EXTRACT | | | | | | |

*Lake color dispersed in xylitol solution
**Calcium carbonate used in place of titanium dioxide
^{a)}All of the active agent is in the coating, which comprises 33% of the product.

The above formulas are used to coat pellets by applying a xylitol/gum arabic solution in multiple coats and air drying. Color or whitener is also mixed in the solution. Chinese hawthorn extract may be dissolved in water or flavor and added between coating applications or mixed with the hot syrup and used in the early stages of coating or used throughout the coating process. After pellets have been coated and dried, talc and wax are added to give a polish.

For coating formulas based on sorbitol, maltitol, lactitol, erythritol, and hydrogenated isomaltulose, gum arabic can be used as a binder and film former, and a crystallization modifier to help facilitate coating. Generally these polyols are more difficult to coat using only a straight syrup, but with proper technique a good smooth hard shell can be made. However, it may be preferable to add a dry charge to quicken the drying process before the pellets get too sticky. The following formulations may be used.

TABLE 30
(DRY WEIGHT PERCENT)

| | EX. 224 | EX. 225 | EX. 226 | EX. 227 | EX. 228 | EX. 229 |
|---|---|---|---|---|---|---|
| MALTITOL | 96.8 | 94.63 | 91.29 | 86.8 | 75.83 | 68.69 |
| MALTITOL POWDER | — | — | — | 10.0 | 20.0 | 25.0 |
| GUM ARABIC | 2.0 | 4.0 | 6.0 | 2.0 | 3.0 | 4.0 |
| FLAVOR | 0.5 | 0.4 | 0.7 | 0.5 | 0.3 | 0.7 |
| TITANUIM DIOXIDE | 0.5 | 0.5 | 1.0 | 0.5 | 0.4 | 0.6 |
| TALC | 0.1 | 0.1 | 0.1 | 0.1 | 0.1 | 0.1 |
| WAX | 0.1 | 0.1 | 0.1 | 0.1 | 0.1 | 0.1 |
| CHINESE HAWTHORN | — | 0.27 | 0.81^{a)} | — | 0.27 | 0.81^{a)} |
| EXTRACT | | | | | | |

^{a)}All of the active agent is in the coating, which comprises 33% of the product.

Maltitol powder is used to dry charge in the early stages of coating. Maltitol, gum arabic, and whitener are blended into a syrup and applied to pellets. After all coating is applied and dried, talc and wax are added to give a polish. Chinese hawthorn extract may be applied in a similar manner as in the previous xylitol coating, or may be preblended with the dry charge material.

In a similar manner, coatings with sorbitol, lactitol, and hydrogenated isomaltulose may be made in the coating formulas in Table 30 by replacing maltitol with any one of the other polyols and maltitol powder with the polyol powder. Like maltitol, the other polyols may become sticky during the coating and drying process, so the dry powder charge may be needed to give the proper drying. In the later stages of the coating process, less gum arabic could be used and a more pure polyol syrup could be used to give a smooth surface. Also, the dry charge would only be used in the early stages of the coating process.

In addition to dry charging with the specific polyol, other ingredients may be added to the dry charge to help absorb moisture. These materials could be inert such as talc, calcium carbonate, magnesium carbonate, starches, gums like Wolfberry extract, gum talha, gum arabic or other moisture absorbing materials. Also, powdered sweeteners or flavors could be added with the dry charge.

Some polyols such as sorbitol, maltitol, lactitol, erythritol, or hydrogenated isomaltulose are not sufficiently sweet compared to sugar or xylitol, so high intensity sweeteners may be added to the coating, such as aspartame, acesulfame K, salts of acesulfame, cyclamate and its salts, saccharin and its salts, alitame, sucralose, thaumatin, monellin, dihydrochalcone, glycyrrhizin, neotame, and combinations thereof. If a hot syrup is applied, heat may degrade the sweetener so only stable sweeteners should be used. Generally high intensity sweeteners are added with the polyol/gum arabic solution to obtain an even distribution in the coatings.

The formulas listed in Table 31 comprise various sugar-type formulas in which active medicament can be added to gum after it is dissolved in water or mixed with various aqueous solvents. Honeysuckle extract is an active medicament used for reducing internal heat or antibacterial effects. These formulas give a 3 gram stick with 30 mg of Honeysuckle extract.

TABLE 31
(WEIGHT PERCENT)

| | EX. 230 | EX. 231 | EX. 232 | EX. 233 | EX. 234 | EX. 235 | EX. 236 | EX. 237 |
|---|---|---|---|---|---|---|---|---|
| SUGAR | 64.6 | 64.0 | 61.0 | 67.0 | 63.0 | 53.0 | 60.7 | 47.0 |
| BASE | 19.2 | 19.2 | 19.2 | 19.2 | 19.2 | 19.2 | 19.2 | 19.2 |
| CORN SYRUP | 12.9 | 10.9 | 8.9 | 2.9 | 6.9 | 6.9 | 0.0 | 2.9 |
| PEPPER-MINT | 0.9 | 0.9 | 0.9 | 0.9 | 0.9 | 0.0 | 0.9 | 0.9 |
| FLAVOR | | | | | | | | |
| GLYCERIN | 1.4 | 0.0 | 0.0 | 0.0 | 0.0 | 0.9 | 1.4 | 0.0 |
| LIQUID/ | 1.0 | 5.0 | 10.0 | 10.0 | 10.0 | 20.0 | 17.8 | 30.0 |
| ACTIVE BLEND | | | | | | | | |

### Example 230

Honeysuckle extract powder can be added directly to the gum.

### Example 231

A 20 gram quantity of Honeysuckle extract can be dissolved in 80 grams of water giving a 20% solution and added to gum.

### Example 232

A 10 gram quantity of Honeysuckle extract can be dissolved in 50 grams of water and mixed with 50 grams of glycerin and added to the gum.

### Example 233

A 10 gram quantity of Honeysuckle extract is mixed with 90 grams of glycerin giving a 10% solution and added to gum.

### Example 234

A 10 gram quantity of Honeysuckle extract is mixed with 90 grams of propylene glycol giving a 10% solution and added to gum.

### Example 235

A 10 gram quantity of Honeysuckle extract is dissolved in 10 grams of peppermint flavor and added to gum.

### Example 236

A 10 grain quantity of Honeysuckle extract is dissolved in 168 grams of corn syrup and added to chewing gum.

### Example 237

To a 200 gram quantity of corn syrup is added 100 grams of glycerin. To this mixture is added 10 gram of Honeysuckle extract and blended. This mixture is then added to gum.

In the next examples of sugar formulations, Honeysuckle extract can be dissolved in water and emulsifiers can be added to the aqueous solution. Example solutions can be prepared by dissolving 20 grams of Honeysuckle extract in 65 grams of water and adding 15 grams of emulsifiers of various hydrophilic-lipophilic balance (HLB) values to the solution. The mixtures can then be used in the following formulas. Example 238 uses a mixture of Honeysuckle extract and water with no emulsifier. The HLB value of the emulsifiers used in Examples 238-243 are listed in Table 32.

TABLE 32
(WEIGHT PERCENT)

| | EX. 238 | EX. 239 | EX. 240 | EX. 241 | EX. 242 | EX. 243 |
|---|---|---|---|---|---|---|
| SUGAR | 50.7 | 50.7 | 50.7 | 50.7 | 50.7 | 50.7 |
| BASE | 19.2 | 19.2 | 19.2 | 19.2 | 19.2 | 19.2 |
| CORN SYRUP | 12.9 | 12.9 | 12.9 | 12.9 | 12.9 | 12.9 |
| GLYCERIN | 1.4 | 1.4 | 1.4 | 1.4 | 1.4 | 1.4 |
| DEXTROSE | 9.9 | 9.9 | 9.9 | 9.9 | 9.9 | 9.9 |
| MONOHY-DRATE | | | | | | |
| COOLING AGENT | 0.1 | — | — | 0.1 | — | 0.2 |
| PEPP. FLAVOR | 0.8 | 0.9 | 0.9 | 0.8 | 0.9 | 0.7 |
| ACTIVE AGENT | 5.0 | 5.0 | 5.0 | 5.0 | 5.0 | 5.0 |
| EMULSIFIER/ | | | | | | |
| WATER MIXTURE | | | | | | |
| | None | HLB = 2 | HLB = 4 | HLB = 6 | HLB = 9 | HLB = 12 |

The formulations made in Examples 238-243 can be changed in that the flavor can be mixed together with the aqueous active agent solution and emulsified before adding the mixture to the gum batch.

The following Tables 33 through 40 are examples of gum formulations that demonstrate formula variations in which Honeysuckle extract may be used. The active agent may be added with or without encapsulation or may be treated for fast release.

Examples 244-247 in Table 33 demonstrate the use of Huang Qi extract in low-moisture sugar formulations showing less than 2% theoretical moisture:

TABLE 33
(WEIGHT PERCENT)

| | EX. 244 | EX. 245 | EX. 246 | EX. 247 |
|---|---|---|---|---|
| SUGAR | 57.9 | 55.9 | 57.9 | 50.9 |
| GUM BASE | 19.2 | 19.2 | 19.2 | 19.2 |
| CORN^{a} SYRUP | 6.0 | 6.0 | — | — |
| DEXTROSE MONOHYDRATE | 10.0 | 10.0 | 10.0 | 10.0 |
| LACTOSE | 0.0 | 0.0 | 0.0 | 5.0 |
| GLYCERIN^{b} | 5.0 | 5.0 | 11.0 | 11.0 |
| FLAVOR | 0.9 | 0.9 | 0.9 | 0.9 |
| HUANG Qi EXTRACT | 1.0 | 3.0 | 1.0 | 3.0 |

^{a}Corn syrup is evaporated to 85% solids, 15% moisture
^{b}Glycerin and syrup may be blended and co-evaporated

Examples 248-251 in Table 34 demonstrate the use of Huang Qui extract in medium-moisture sugar formulations having about 2% to about 5% moisture.

Examples 252-255 in Table 35 demonstrate the use of Huang Qui extract in high-moisture sugar formulations having more than about 5% moisture.

TABLE 34
(WEIGHT PERCENT)

| | EX. 248 | EX. 249 | EX. 250 | EX. 251 |
|---|---|---|---|---|
| SUGAR | 52.5 | 50.5 | 52.5 | 49.0 |
| GUM BASE | 19.2 | 19.2 | 19.2 | 19.2 |
| CORN SYRUP^{a} | 15.0 | 15.0 | 13.0 | 12.5 |
| DEXTROSE MONOHYDRATE | 10.0 | 10.0 | 10.0 | 10.0 |
| GLYCERIN^{b} | 1.4 | 1.4 | 3.4 | 5.4 |
| FLAVOR | 0.9 | 0.9 | 0.9 | 0.9 |
| HUANG Qi EXTRACT | 1.0 | 3.0 | 1.0 | 3.0 |

^{a}Corn syrup is evaporated to 85% solids, 15% moisture
^{b}Glycerin and syrup may be blended and co-evaporated

TABLE 35
(WEIGHT PERCENT)

| | EX. 252 | EX. 253 | EX. 254 | EX. 255 |
|---|---|---|---|---|
| SUGAR | 50.0 | 48.0 | 49.0 | 47.0 |
| GUM BASE | 24.0 | 24.0 | 24.0 | 24.0 |
| CORN SYRUP | 24.0 | 24.0 | 24.0 | 24.6 |
| GLYCERIN | 0.0 | 0.0 | 1.0 | 0.4 |
| FLAVOR | 1.0 | 1.0 | 1.0 | 1.0 |
| HUANG Qi EXTRACT | 1.0 | 3.0 | 1.0 | 3.0 |

Examples 256-259 in Table 36 and Examples 260-267 in Tables 37 and 38 demonstrate the use of Loquat Leaf extract in low- and high-moisture gums that are sugar-free. Low-moisture gums have less than about 2% moisture, and high-moisture gums have greater than 2% moisture.

TABLE 36
(WEIGHT PERCENT)

| | EX. 256 | EX. 257 | EX. 258 | EX. 259 |
|---|---|---|---|---|
| BASE | 25.5 | 25.4 | 25.5 | 25.2 |
| SORBITOL | 50.0 | 48.0 | 48.0 | 43.0 |
| MANNITOL | 12.0 | 12.0 | 12.0 | 12.0 |
| GLYCERIN | 10.0 | 10.0 | 12.0 | 15.0 |
| COOLING AGENT | 0.1 | 0.1 | — | 0.3 |
| FLAVOR | 1.4 | 1.5 | 1.5 | 1.5 |
| LOQUAT LEAF EXTRACT | 1.0 | 3.0 | 1.0 | 3.0 |

TABLE 37
(WEIGHT PERCENT)

| | EX. 260 | EX. 261 | EX. 262 | EX. 263 |
|---|---|---|---|---|
| BASE | 25.5 | 25.5 | 25.5 | 25.5 |
| SORBITOL | 50.0 | 48.0 | 40.0 | 38.0 |
| LIQUID SORBITOL* | 10.0 | 10.0 | 20.0 | 20.0 |
| MANNITOL | 10.0 | 10.0 | 10.0 | 10.0 |
| GLYCERIN | 2.0 | 2.0 | 2.0 | 2.0 |
| FLAVOR | 1.5 | 1.5 | 1.5 | 1.5 |
| LOQUAT LEAF EXTRACT | 1.0 | 3.0 | 1.0 | 3.0 |

*Sorbitol liquid contains 70% sorbitol, 30% water

TABLE 38
(WEIGHT PERCENT)

| | EX. 264 | EX. 265 | EX. 266 | EX. 267 |
|---|---|---|---|---|
| BASE | 25.5 | 25.5 | 25.5 | 25.5 |
| SORBITOL | 50.0 | 46.0 | 44.0 | 42.0 |
| HSH SYRUP* | 10.0 | 10.0 | 10.0 | 10.0 |
| MANNITOL | 8.0 | 8.0 | 8.0 | 8.0 |
| GLYCERIN** | 4.0 | 6.0 | 10.0 | 10.0 |
| FLAVOR | 1.5 | 1.5 | 1.5 | 1.5 |
| LOQUAT LEAF EXTRACT | 1.0 | 3.0 | 1.0 | 3.0 |

*Hydrogenated starch hydrolyzate syrup
**Glycerin and HSH syrup may be blended or co-evaporated

Table 39 shows sugar chewing formulations that can be made with various types of sugars.

TABLE 39
(WEIGHT PERCENT)

| | EX. 268 | EX. 269 | EX. 270 | EX. 271 | EX. 272 | EX. 273 |
|---|---|---|---|---|---|---|
| GUM BASE | 19.2 | 19.2 | 19.1 | 19.2 | 19.2 | 19.2 |
| SUCROSE | 48.5 | 44.5 | 43.5 | 39.5 | 33.5 | 39.5 |
| GLYCERIN | 1.4 | 3.4 | 1.4 | 3.4 | 1.4 | 3.4 |
| CORN SYRUP | 14.0 | 14.0 | 14.0 | 14.0 | 14.0 | 14.0 |
| DEXTROSE | 5.0 | 5.0 | — | — | 10.0 | 5.0 |
| LACTOSE | 5.0 | 5.0 | 10.0 | 10.0 | — | — |
| FRUCTOSE | 5.0 | 5.0 | 10.0 | 10.0 | 10.0 | 5.0 |
| INVERT SUGAR | — | — | — | — | 10.0 | 10.0 |
| MALTOSE | — | — | — | — | — | — |
| CORN SYRUP SOLIDS | — | — | — | — | — | — |
| COOLING AGENT | — | — | 0.1 | 0.2 | | 0.1 |
| PEPPERMINT FLAVOR | 0.9 | 0.9 | 0.9 | 0.7 | 0.9 | 0.8 |
| LUO HAN GUO | 1.0 | 3.0 | 1.0 | 3.0 | 1.0 | 3.0 |
| EXTRACT | | | | | | |
| | EX. 274 | EX. 275 | EX. 276 | EX. 277 | EX. 278 | EX. 279 |
| GUM BASE | 19.2 | 19.2 | 19.2 | 19.2 | 19.2 | 19.2 |
| SUCROSE | 33.5 | 39.5 | 33.5 | 39.5 | 41.5 | 42.5 |
| GLYCERIN | 1.4 | 3.4 | 1.4 | 3.4 | 1.4 | 3.4 |
| CORN SYRUP | 14.0 | 14.0 | 14.0 | 14.0 | 11.0 | 11.0 |
| DEXTROSE | 10.0 | 5.0 | 10.0 | 5.0 | 10.0 | 5.0 |
| LACTOSE | — | — | — | — | — | — |
| FRUCTOSE | 10.0 | 5.0 | 10.0 | 5.0 | 5.0 | 5.0 |
| INVERT SUGAR | 10.0 | 10.0 | — | — | 5.0 | 5.0 |
| MALTOSE | — | — | 10.0 | 10.0 | — | — |
| CORN SYRUP SOLIDS | — | — | — | — | 5.0 | 5.0 |
| FRUIT FLAVOR | 0.9 | — | 0.1 | — | — | — |
| PEPPERMINT FLAVOR | 0.9 | 0.9 | 0.9 | 0.9 | 0.9 | 0.9 |
| LUO HAN GUO | 1.0 | 3.0 | 1.0 | 3.0 | 1.0 | 3.0 |
| EXTRACT | | | | | | |

Table 40 shows chewing gum formulations that are free of sugar. These formulations can use a wide variety of other non-sugar alditols.

TABLE 40
(WEIGHT PERCENT)

| | EX. 280 | EX. 281 | EX. 282 | EX. 283 | EX. 284 | EX. 285 |
|---|---|---|---|---|---|---|
| GUM BASE | 25.5 | 25.5 | 25.5 | 25.5 | 25.5 | 25.5 |
| GLYCERIN | 8.0 | 8.0 | 8.0 | 8.0 | 8.0 | 2.0 |
| SORBITOL | 47.0 | 35.0 | 37.0 | 30.0 | 31.0 | 27.0 |
| MANNITOL | — | 10.0 | 10.0 | 10.0 | 10.0 | 6.0 |
| SORBITOL LIQUID | 17.0 | 17.0 | — | — | — | — |
| LYCASIN | — | — | 17.0 | 12.0 | 8.0 | 20.0 |
| MALTITOL | — | — | — | 10.0 | — | — |
| XYLITOL | — | — | — | — | 15.0 | 15.0 |
| LACTITOL | — | — | — | — | — | — |
| PALATINIT | — | — | — | — | — | — |
| FLAVOR | 1.5 | 1.5 | 1.5 | 1.5 | 1.5 | 1.5 |
| LUO HAN GUO | 1.0 | 3.0 | 1.0 | 3.0 | 1.0 | 3.0 |
| EXTRACT | | | | | | |
| | EX. 286 | EX. 287 | EX. 288 | EX. 289 | EX. 290 | EX. 291 |
| GUM BASE | 25.5 | 25.5 | 25.5 | 25.5 | 25.3 | 25.5 |
| GLYCERIN | 8.0 | 8.0 | 8.0 | 2.0 | 8.0 | 2.0 |
| SORBITOL | 41.0 | 34.0 | 31.0 | 38.0 | 29.0 | 38.0 |
| MANNITOL | 8.0 | 8.0 | 8.0 | — | — | — |
| SORBITOL LIQUID | 5.0 | — | — | — | — | — |
| LYCASIN | — | 5.0 | 5.0 | 5.0 | 10.0 | 20.0 |
| MALITITOL | — | 5.0 | — | — | — | — |
| XYLITOL | — | — | — | 15.0 | 15.0 | — |
| LACTITOL | 10.0 | 10.0 | 10.0 | — | — | — |
| PALATINIT | — | — | 10.0 | 10.0 | 10.0 | 10.0 |
| COOLING AGENT | — | — | 0.1 | — | 0.2 | — |
| FLAVOR | 1.5 | 1.5 | 1.4 | 1.5 | 1.5 | 1.5 |
| LUO HAN GUO | 1.0 | 3.0 | 1.0 | 3.0 | 1.0 | 3.0 |
| EXTRACT | | | | | | |

High-intensity sweeteners (HIS) such as aspartame, acesulfame K, or the salts of acesulfame, cyclamate and its salts, saccharin and its salts, alitame, neotame, sucralose, thaumatin, monellin, dihydrochalcone, stevioside, glycyrrhizin, and combinations thereof may be used in any of the Examples listed in Tables 33-40. Since Honeysuckle extract may reduce sweetness, HIS may be used in sugar gum, and some of the alditols in sugar-free gum are less sweet than sugar so higher levels of HIS may be needed to obtain the proper level of sweetness.

High-intensity sweeteners (HIS) may also be modified to control their release in those chewing gum formulations. This can be controlled by various methods of encapsulation, agglomeration, absorption, or a combination of methods to obtain either a fast or slow release of the sweetener. Sweetener combinations, some of which may be synergistic, may also be included in the gum formulations.

As noted earlier, the gum formulas can be prepared as stick or tab products in the sugar or sugarless type formulations. These formulas can also be made in a pellet or pillow shape pellet or a round ball or any other shape of product for coating/panning. However, gum formulas are generally adjusted to a higher level of gum base to give a more consumer acceptable size of gum bolus.

As a result, gum centers are usually formulated with about 25% to about 40% gum base with a corresponding decrease in the other ingredients except flavor. Generally flavors increase with the level of gum base as the base tends to bind flavors into the gum and more flavor is needed to give a good flavorful product. However flavors can also be added to the coating to give increased flavor impact and more flavor perception.

Some typical sugar type gum center formulations are shown in Table 41 containing Honeysuckle extract, which is used to reduce internal heat.

TABLE 41
(WEIGHT PERCENT)

| | EX. 292 | EX. 293 | EX. 294 | EX. 295 | EX. 296 | EX. 297 |
|---|---|---|---|---|---|---|
| SUGAR | 52.0 | 48.0 | 46.5 | 44.0 | 40.0 | 37.5 |
| GUM BASE | 26.0 | 30.0 | 35.0 | 26.0 | 30.0 | 35.0 |
| CORN SYRUP | 20.0 | 19.0 | 15.00 | 18.0 | 17.0 | 14.00 |
| GLYCERIN | 1.0 | 1.0 | 1.00 | 1.0 | 1.0 | 1.00 |
| PEPPERMINT FLAVOR | 1.0 | 1.0 | 1.00 | 1.0 | 1.0 | 1.00 |
| DEXTROSE | — | — | — | 10.0 | 10.0 | 10.00 |
| MONOHYDRATE | | | | | | |
| HONEYSUCKLE | —^{a)} | 1.0 | 1.5 | —^{a)} | 1.0 | 1.5 |
| EXTRACT | | | | | | |

^{a)}All of the active agent is in the coating, which comprises 33% of the product.

Formulations with or without active Honeysuckle extract can also be made similar to those found in Tables 33-38 for low, medium, and high moisture formulas. Higher levels of base may be used with a corresponding decrease in other ingredients. Also, other sugars are polyols may be used in the gum center as found in Tables 39 and 40. Honeysuckle extract may be added to a gum center only, or to a gum coating with none in the center, or to both center and coating. Coated gum pieces are about 1.5 grams per piece, so to obtain 30 mg of Honeysuckle extract in two gum pieces, total piece must contain 1.0%.

Honeysuckle extract can be used in the coating formula on the various pellet gum formulations. The following Table 42 shows some sugar and dextrose type formulas:

TABLE 42
(DRY WEIGHT PERCENT)

| | EX. 298 | EX. 299 | EX. 300 | EX. 301 | EX. 302 | EX. 303 |
|---|---|---|---|---|---|---|
| SUGAR | 97.1 | 94.4 | 91.1 | 96.9 | 94.1 | 90.6 |
| GUM ARABIC | 2.0 | 3.0 | 4.0 | 2.0 | 3.0 | 4.0 |
| TITANIUM DIOXIDE | 0.5 | 1.0 | 1.0 | — | — | — |
| CALCIUM CARBONATE | — | — | — | 0.5 | 1.0 | 2.0 |
| FLAVOR | 0.3 | 0.5 | 0.8 | 0.5 | 0.8 | 0.3 |
| WAX | 0.1 | 0.1 | 0.1 | 0.1 | 0.1 | 0.1 |
| HONEYSUCKLE | — | 1.0 | 3.0^{a)} | — | 1.0 | 3.0^{a)} |
| EXTRACT | | | | | | |

| | EX. 304 | EX. 305 | EX. 306 | EX. 307 |
|---|---|---|---|---|
| DEXTROSE MONOHYDRATE | 97.6 | 94.4 | 96.2 | 91.5 |
| GUM ARABIC | 1.5 | 3.0 | 1.5 | 3.0 |
| TITANIUM DIOXIDE | 0.5 | 1.0 | — | — |
| CALCIUM CARBONATE | — | — | 1.0 | 2.0 |
| FLAVOR | 0.3 | 0.5 | 0.2 | 0.4 |
| WAX | 0.1 | 0.1 | 0.1 | 0.1 |
| HONEYSUCKLE EXTRACT | — | 1.0 | 1.0 | 3.0^{a)} |

^{a)}All of the active agent is in the coating, which comprises 33% of the product.

The above formulations are made by making a syrup by dissolving the sugar and gum arabic in solution at about 75% solids at boiling, and suspending titanium dioxide or calcium carbonate in this syrup. Honeysuckle extract may be dissolved in water, not mixed with hot syrup, but applied between coatings, or it may be added to the hot syrup and used in the early stages of coating or used throughout the coating process. Flavor is not mixed with the hot syrup, but added at low levels with one or more coats. Honeysuckle extract may be dissolved in flavor and added to the coating. After the final coats are applied and dried, wax is applied to give a smooth polish.

As shown in Table 43, some of the sugar or dextrose may be added as a dry charge, which may also contain the active.

TABLE 43
(DRY WEIGHT PERCENT)

| | EX. 308 | EX. 309 | EX. 310 | EX. 311 | EX. 312 | EX. 313 |
|---|---|---|---|---|---|---|
| SUGAR | 76.5 | 78.4 | — | — | 86.5 | — |
| DEXTROSE MONOHYDRATE | — | — | 76.5 | 83.3 | — | 84.1 |
| POWDER SUGAR* | 20.0 | 15.0 | — | — | — | — |
| POWDER DEXTROSE* | — | — | 20.0 | 10.0 | — | — |
| GUM ARABIC POWDER | 2.0 | 3.0 | 2.0 | 3.0 | 8.0 | 8.0 |
| GUM ARABIC SOLUTION | — | — | — | — | 4.0 | 4.0 |
| FLAVOR | 0.4 | 0.5 | 0.4 | 0.6 | 0.4 | 0.8 |
| WAX | 0.1 | 0.1 | 0.1 | 0.1 | 0.1 | 0.1 |
| HONEYSUCKLE EXTRACT | 1.0 | 3.0^{a)} | 1.0 | 3.0^{a)} | 1.0 | 3.0^{a)} |

*Powder and/or crystalline sugar may be used.
^{a)}All of the active agent is in the coating, which comprises 33% of the product.

In Examples 308-311 gum arabic powder is blended in the sugar syrup. In Examples 312 and 313, gum arabic powder is dry charged after a gum arabic solution is applied in the first stages of coating, then this is followed by a hard shell coating of sugar solution or dextrose solution.

Honeysuckle extract may also be used in coating of sugarless gum centers. Like sugar gum centers, the base formulation can be increased in proportion to the amount of coating applied to the center. Formulations with and without Honeysuckle extract similar to those found in Tables 33-38 for low and high moisture gum can be used to make gum centers. Generally, the base level may be increased to 30-46% with the other ingredients proportionally reduced. Some typical gum formulas are in Table 44.

TABLE 44
(WEIGHT PERCENT)

| | EX. 314 | EX. 315 | EX. 316 | EX. 317 | EX. 318 | EX. 319 | EX. 320 |
|---|---|---|---|---|---|---|---|
| GUM BASE | 35.0 | 35.0 | 30.0 | 30.0 | 30.0 | 40.0 | 50.0 |
| CALCIUM CARBONATE | — | — | 5.0 | 10.0 | 15.0 | — | — |
| SORBITOL | 43.3 | 44.3 | 44.8 | 40.3 | 43.8 | 40.2 | 24.5 |
| MANNITOL | 10.0 | 10.0 | 5.0 | 10.0 | — | 8.0 | 10.0 |
| GLYCERIN | — | 8.0 | 2.0 | — | 8.0 | 2.0 | 2.0 |
| SORBITOL LIQUID | 10.0 | — | 10.0 | 8.0 | — | 6.0^{a)} | 10.0^{a)} |
| FLAVOR | 1.5 | 1.5 | 1.5 | 1.5 | 2.0 | 2.0 | 1.3 |
| HIGH INTENSITY | 0.2 | 0.2 | 0.2 | 0.2 | 0.2 | 0.3 | 0.2 |
| SWEETENER | | | | | | | |
| HONEYSUCKLE | —^{c)} | 1.0 | 1.5 | —^{c)} | 1.0 | 1.5 | 2.0^{d)} |
| EXTRACT^{b)} | | | | | | | |

^{a)}Lycasin brand hydrogenated starch hydrolyzate used instead of sorbitol liquid
^{b)}This material may be dissolved in water, glycerin, sorbitol liquid, or HSH.
^{c)}All of the active agent is in the coating, which comprises 33% of the product.
^{d)}This example required 50% of the product to be a coating with no active agent in the coating, to give a gum product with 1% active agent.

In the above center formulations, the high intensity sweetener used is aspartame. However other high intensity such as alitame, acesulfame K, salts of acesulfame, cyclamate and its salts, saccharin and its salts, neotame, sucralose, thaumatin, monellin, dihydrochalcone, stevioside, glycyrrhizin and combinations thereof may be used in any of the examples with the level adjusted for sweetness.

Lycasin and other polyols such as maltitol, erythritol, xylitol, lactitol and hydrogenated isomaltulose may also be used in the gum center formulations at various levels similar to those shown in Table 40. The texture may be adjusted by varying glycerin or sorbitol liquid. Sweetness of the center formulation can also be adjusted by varying the level of high intensity sweetener.

Honeysuckle extract may be used in sugarless coatings with xylitol, sorbitol, maltitol, lactitol, hydrogenated isomaltulose and erythritol. The following table gives formulas for a xylitol coating:

TABLE 45
(DRY WEIGHT PERCENT)

| | EX. 321 | EX. 322 | EX. 323 | EX. 324 | EX. 325 | EX. 326 |
|---|---|---|---|---|---|---|
| XYLITOL | 94.8 | 91.4 | 87.6 | 90.1 | 88.7 | 85.8 |
| GUM ARABIC | 4.0 | 6.0 | 7.0 | 8.5 | 8.5 | 10.0 |
| FLAVOR | 0.5 | 0.5 | 0.7 | 0.7 | 0.9 | 0.5 |
| TITANIUM DIOXIDE | 0.5 | 0.9 | — | 0.5 | 0.5** | 0.5** |
| TALC | 0.1 | 0.1 | 0.1 | 0.1 | 0.1 | 0.1 |
| COOLING AGENT | — | — | 0.1 | — | 0.2 | — |
| WAX | 0.1 | 0.1 | 0.1 | 0.1 | 0.1 | 0.1 |
| COLOR* | — | — | 1.4 | — | — | — |
| HONEYSUCKLE EXTRACT | — | 1.0 | 3.0^{a)} | — | 1.0 | 3.0^{a)} |

*Lake color dispersed in xylitol solution
**Calcium carbonate used in place of titanium dioxide
^{a)}All of the active agent is in the coating, which comprises 33% of the product.

The above formulas are used to coat pellets by applying a xylitol/gum arabic solution in multiple coats and air drying. Color or whitener is also mixed in the solution. Honeysuckle extract may be dissolved in water or flavor and added between coating applications or mixed with the hot syrup and used in the early stages of coating or used throughout the coating process. After pellets have been coated and dried, talc and wax are added to give a polish.

For coating formulas based on sorbitol, maltitol, lactitol, erythritol, and hydrogenated isomaltulose, gum arabic can be used as a binder and film former, and a crystallization modifier to help facilitate coating. Generally these polyols are more difficult to coat using only a straight syrup, but with proper technique a good smooth hard shell can be made. However, it may be preferable to add a dry charge to quicken the drying process before the pellets get too sticky. The following formulations may be used.

TABLE 46
(DRY WEIGHT PERCENT)

| | EX. 327 | EX. 328 | EX. 329 | EX. 330 | EX. 331 | EX. 332 |
|---|---|---|---|---|---|---|
| MALTITOL | 96.8 | 93.9 | 89.1 | 86.8 | 75.1 | 66.5 |
| MALTITOL | — | — | — | 10.0 | 20.0 | 25.0 |
| POWDER | | | | | | |
| GUM ARABIC | 2.0 | 4.0 | 6.0 | 2.0 | 3.0 | 4.0 |
| FLAVOR | 0.5 | 0.4 | 0.7 | 0.5 | 0.3 | 0.7 |
| TITANIUM | 0.5 | 0.5 | 1.0 | 0.5 | 0.4 | 0.6 |
| DIOXIDE | | | | | | |
| TALC | 0.1 | 0.1 | 0.1 | 0.1 | 0.1 | 0.1 |
| WAX | 0.1 | 0.1 | 0.1 | 0.1 | 0.1 | 0.1 |
| HONEY- | — | 1.0 | 3.0^{a)} | — | 1.0 | 3.0^{a)} |
| SUCKLE | | | | | | |
| EXTRACT | | | | | | |

^{a)}All of the active agent is in the coating, which comprises 33% of the product.

Maltitol powder is used to dry charge in the early stages of coating. Maltitol, gum arabic, and whitener are blended into a syrup and applied to pellets. After all coating is applied and dried, talc and wax are added to give a polish. Honeysuckle extract may be applied in a similar manner as in the previous xylitol coating examples, or may be preblended with the dry charge material.

In a similar manner, coatings with sorbitol, lactitol, and hydrogenated isomaltulose may be made in the coating formulas in Table 46 by replacing maltitol with any one of the other polyols and maltitol powder with the polyol powder. Like maltitol, the other polyols may become sticky during the coating and drying process, so the dry powder charge may be needed to give the proper drying. In the later stages of the coating process, less gum arabic could be used and a more pure polyol syrup could be used to give a smooth surface. Also, the dry charge would only be used in the early stages of the coating process.

In addition to dry charging with the specific polyol, other ingredients may be added to the dry charge to help absorb moisture. These materials could be inert such as talc, calcium carbonate, magnesium carbonate, starches, gums like arabinogalactan, gum talha, gum arabic or other moisture absorbing materials. Also, powdered sweeteners or flavors could be added with the dry charge.

Some polyols such as sorbitol, maltitol, lactitol, erythritol, or hydrogenated isomaltulose are not sufficiently sweet compared to sugar or xylitol, so high intensity sweeteners may be added to the coating, such as aspartame, acesulfame K, salts of acesulfame, cyclamate and its salts, saccharin and its salts, alitame, sucralose, thaumatin, monellin, dihydrochalcone, glycyrrhizin, neotame, and combinations thereof. If a hot syrup is applied, heat may degrade the sweetener so only stable sweeteners should be used. Generally high intensity sweeteners are added with the polyol/gum arabic solution to obtain an even distribution in the coatings.

Liquid flavors generally are not added throughout the coating but at specific points throughout the process. When flavor is added, less air is used for drying until the flavor coating is covered by the next coatings and dried. Flavors may be various spearmint, peppermint, wintergreen, cinnamon, and fruit flavors to yield a wide variety of flavored chewing gum products.

As noted earlier, the gum formulas can be prepared as stick or tab products in the sugar or sugarless type formulations. These formulas can also be made in a pellet or pillow shape pellet or a round ball or any other shape of product for coating/panning. However, gum formulas are generally adjusted to a higher level of gum base to give a more consumer acceptable size of gum bolus.

As a result, gum centers are usually formulated with about 25% to about 40% gum base with a corresponding decrease in the other ingredients except flavor. Generally flavors increase with the level of gum base as the base tends to bind flavors into the gum and more flavor is needed to give a good flavorful product. However flavors can also be added to the coating to give increased flavor impact and more flavor perception.

Some typical sugar type gum center formulations are shown in Table 47 in which ginger powder or oil to settle upset stomachs and help digestion. These formulas give a 1.5 gram piece containing 5 mg of ginger powder or 0.33%. Gum center formulas may or may not contain ginger powder which has been encapsulated for controlled release.

TABLE 47
(WEIGHT PERCENT)

| | EX. 333 | EX. 334 | EX. 335 | EX. 336 | EX. 337 | EX. 338 |
|---|---|---|---|---|---|---|
| SUGAR | 52.0 | 48.67 | 47.5 | 44.0 | 40.67 | 38.5 |
| GUM BASE | 26.0 | 30.0 | 35.0 | 26.0 | 30.0 | 35.0 |
| CORN SYRUP | 20.0 | 19.0 | 15.0 | 18.0 | 17.0 | 14.0 |
| GLYCERIN | 1.0 | 1.0 | 1.0 | 1.0 | 1.0 | 1.0 |
| PEPPERMINT FLAVOR | 1.0 | 1.0 | 1.0 | 1.0 | 1.0 | 1.0 |
| DEXTROSE | — | — | — | 10.0 | 10.0 | 10.0 |
| MONOHYDRATE | | | | | | |
| GINGER POWDER | —^{a)} | 0.33 | 0.5 | —^{a)} | 0.33 | 0.5 |

^{a)}All of the active agent is in the coating, which comprises 33% of the product

Formulations with or without Ginger powder can also be made similar to those found in previous tables for low, medium, and high moisture formulas. Higher levels of base may be used with a corresponding decrease in other ingredients. Also, other sugars and polyols may be used in the gum center as found in previous tables. Ginger powder may be added to a gum center only, into a gum coating with more in the center or to both center and coating.

Ginger powder can be used in the coating formula on the various pellet gum formulations. The following Table 48 shows some sugar and dextrose type formulas:

TABLE 48
(DRY WEIGHT PERCENT)

| | EX. | EX. | EX. | EX. | EX. | EX. |
|---|---|---|---|---|---|---|
| | 339 | 340 | 341 | 342 | 343 | 344 |
| SUGAR | 97.1 | 95.07 | 93.1 | 96.9 | 94.77 | 92.6 |
| GUM ARABIC | 2.0 | 3.0 | 4.0 | 2.0 | 3.0 | 4.0 |
| TITANIUM | 0.5 | 1.0 | 1.0 | — | — | — |
| DIOXIDE | | | | | | |
| CALCIUM | — | — | — | 0.5 | 1.0 | 2.0 |
| CARBONATE | | | | | | |
| FLAVOR | 0.3 | 0.5 | 0.8 | 0.5 | 0.8 | 0.3 |
| WAX | 0.1 | 0.1 | 0.1 | 0.1 | 0.1 | 0.1 |
| GINGER | — | 0.33 | 1.0^{a)} | — | 0.33 | 1.0^{(a} |
| POWDER | | | | | | |

| | EX. | EX. | EX. | EX. |
|---|---|---|---|---|
| | 345 | 346 | 347 | 348 |
| DEXTROSE MONOHYDRATE | 97.6 | 95.07 | 96.87 | 93.5 |
| GUM ARABIC | 1.5 | 3.0 | 1.5 | 3.0 |
| TITANIUM DIOXIDE | 0.5 | 1.0 | — | — |
| CALCIUM CARBONATE | — | — | 1.0 | 2.0 |
| FLAVOR | 0.3 | 0.5 | 0.2 | 0.4 |
| WAX | 0.1 | 0.1 | 0.1 | 0.1 |
| GINGER POWDER | — | 0.33 | 0.33 | 1.0^{(a} |

^{a)}All of the active agent is in the coating, which comprises 33% of the product.

The above formulations are made by making a syrup by dissolving the sugar and gum arabic in solution at about 75% solids at boiling, and suspending titanium dioxide or calcium carbonate in this syrup. Ginger powder may be dissolved in water, not mixed with hot syrup, but applied between coatings, or it may be added to the hot syrup and used in the early stages of coating or used throughout the coating process. Flavor is not mixed with the hot syrup, but added at low levels with one or more coats. Ginger powder or oil may also be premixed with the flavor. After the final coats are applied and dried, wax is applied to give a smooth polish.

The above process gives a hard shell coating. Often a dry charge of powdered sugar or dextrose monohydrate may be used. This gives a somewhat softer coating. A dry charge may be used to build up a coating, but then finished with a straight syrup to obtain a hard shell. Ginger powder may be added dry to the coating with the dry charge material. Table 49 gives these types of formulas.

TABLE 49
(DRY WEIGHT PERCENT)

| | EX. | EX. | EX. | EX. | EX. | EX. |
|---|---|---|---|---|---|---|
| | 349 | 350 | 351 | 352 | 353 | 354 |
| SUGAR | 77.16 | 80.4 | — | — | 87.17 | — |
| DEXTROSE | — | — | 77.16 | 85.3 | — | 86.1 |
| MONOHYDRATE | | | | | | |
| POWDER SUGAR* | 20.0 | 15.0 | — | — | — | — |
| POWDER | — | — | 20.0 | 10.0 | — | — |
| DEXTROSE* | | | | | | |
| GUM ARABIC | 2.0 | 3.0 | 2.0 | 3.0 | 8.0 | 8.0 |
| POWDER | | | | | | |
| GUM ARABIC | — | — | — | — | 4.0 | 4.0 |
| SOLUTION | | | | | | |
| COOLING AGENT | 0.1 | — | 0.1 | — | 0.1 | — |
| FLAVOR | 0.4 | 0.5 | 0.4 | 0.6 | 0.3 | 0.8 |
| WAX | 0.1 | 0.1 | 0.1 | 0.1 | 0.1 | 0.1 |
| GINGER POWDER | 0.33 | 1.0^{a)} | 0.33 | 1.0^{a)} | 0.33 | 1.0^{a)} |

*Powder and/or crystalline sugar may be used.
^{a)}All of the active agent is in the coating, which comprises 33% of the product.

In Examples 349-352, gum arabic is blended in the sugar syrup. In Examples 353 and 354, gum arabic powder is dry charged after gum arabic solution is applied in the first stages of coating, then this is followed by a hard shell coating of sugar solution or dextrose solution.

Ginger powder or oil may also be used in coating of sugarless gum centers. Like sugar gum centers, the base formulation can be increased in proportion to the amount of coating applied to the center. Formulations with and without ginger powder chloride similar to those found in previous tables for low and high moisture gum can be used to make gum centers. Generally, the base level may be increased to 30-46% with the other ingredients proportionally reduced. Some typical gum center formulas are in Table 50.

TABLE 50
(WEIGHT PERCENT)

| | EX. | EX. | EX. | EX. | EX. | EX. | EX. |
|---|---|---|---|---|---|---|---|
| | 355 | 356 | 357 | 358 | 359 | 360 | 361 |
| GUM BASE | 35.0 | 35.0 | 30.0 | 30.0 | 30.0 | 40.0 | 50.0 |
| CALCIUM | — | — | 5.0 | 10.0 | 15.0 | — | — |
| CARBONATE | | | | | | | |
| SORBITOL | 43.3 | 44.97 | 45.8 | 40.3 | 44.47 | 41.2 | 25.84 |
| MANNITOL | 10.0 | 10.0 | 5.0 | 10.0 | — | 8.0 | 10.0 |
| GLYCERIN | — | 8.0 | 2.0 | — | 8.0 | 2.0 | 2.0 |
| SORBITOL | 10.0 | — | 10.0 | 8.0 | — | 6.0^{a)} | 10.0^{a)} |
| LIQUID | | | | | | | |
| FLAVOR | 1.5 | 1.5 | 1.5 | 1.5 | 2.0 | 2.0 | 1.3 |
| HIGH | 0.2 | 0.2 | 0.2 | 0.2 | 0.2 | 0.3 | 0.2 |
| INTENSITY | | | | | | | |
| SWEETENER | | | | | | | |
| GINGER | —^{c)} | 0.33 | 0.5 | —^{c)} | 0.33 | 0.5 | 0.66^{d)} |
| POWDER or | | | | | | | |
| OIL^{b)} | | | | | | | |

^{a)}Lycasin brand hydrogenated starch hydrolyzate used instead of sorbitol liquid.
^{b)}This material may be dissolved/suspended in water, glycerin, sorbitol liquid, flavor oils or HSH.
^{c)}All of the active agent is in the coating, which comprises 33% of the product.
^{d)}This example requires 50% of the product to be a coating with no active agent in the coating, to give a gum product with 0.33% active.

In the above center formulations, the high intensity sweetener used is aspartame. However other high intensity such as alitame, acesulfame K, salts of acesulfame, cyclamate and its salts, saccharin and its salts, neotame, sucralose, thaumatin, monellin, dihydrochalcone, stevioside, glycyrrhizin and combinations thereof may be used in any of the examples with the level adjusted for sweetness.

Lycasin and other polyols such as maltitol, xylitol, erythritol, lactitol and hydrogenated isomaltulose may also be used in the gum center formulations at various levels. The texture may be adjusted by varying glycerin or sorbitol liquid. Sweetness of the center formulation can also be adjusted by varying the level of high intensity sweetener.

Ginger powder or oil may be used in sugarless coatings with xylitol, sorbitol, maltitol, lactitol, hydrogenated isomaltulose and erythritol. The following table gives formulas for a xylitol coating:

TABLE 51
(DRY WEIGHT PERCENT)

| | EX. 362 | EX. 363 | EX. 364 | EX. 365 | EX. 366 | EX. 367 |
|---|---|---|---|---|---|---|
| XYLITOL | 94.8 | 92.07 | 89.7 | 90.1 | 89.57 | 87.8 |
| GUM ARABIC | 4.0 | 6.0 | 7.0 | 8.5 | 8.5 | 10.0 |
| FLAVOR | 0.5 | 0.5 | 0.7 | 0.7 | 0.9 | 0.5 |
| TITANIUM | 0.5 | 0.9 | — | 0.5 | 0.5** | 0.5** |
| DIOXIDE | | | | | | |
| TALC | 0.1 | 0.1 | 0.1 | 0.1 | 0.1 | 0.1 |
| WAX | 0.1 | 0.1 | 0.1 | 0.1 | 0.1 | 0.1 |
| COLOR* | — | — | 1.4 | — | — | — |
| GINGER | — | 0.33 | 1.0^{a)} | — | 0.33 | 1.0^{a)} |
| POWDER | | | | | | |

*Lake color dispersed in xylitol solution
**Calcium carbonate used in place of titanium dioxide
^{a)}All of the active agent is in the coating, which comprises 33% of the product.

The above formulas are used to coat pellets by applying a xylitol/gum arabic solution in multiple coats and air drying. Color or whitener is also mixed in the solution. Ginger powder or oil may be dissolved/suspended in water or flavor and added between coating applications, or mixed with the hot syrup and used in the early stages of coating or used throughout the coating process. After pellets have been coated and dried, talc and wax are added to give a polish.

For coating formulas based on sorbitol, maltitol, lactitol, erythritol, and hydrogenated isomaltulose, gum arabic can be used as a binder and film former, and a crystallization modifier to help facilitate coating. Generally these polyols are more difficult to coat using only a straight syrup, but with proper technique a good smooth hard shell can be made. However, it may be preferable to add a dry charge to quicken the drying process before the pellets get too sticky. The following formulations may be used.

TABLE 52
(DRY WEIGHT PERCENT)

| | EX. 368 | EX. 369 | EX. 370 | EX. 371 | EX. 372 | EX. 373 |
|---|---|---|---|---|---|---|
| MALTITOL | 96.8 | 94.57 | 91.1 | 86.8 | 75.77 | 68.5 |
| MALTITOL | — | — | — | 10.0 | 20.0 | 25.0 |
| POWDER | | | | | | |
| ARABINO- | 2.0 | 4.0 | 6.0 | 2.0 | 3.0 | 4.0 |
| GALACTAN | | | | | | |
| FLAVOR | 0.5 | 0.4 | 0.7 | 0.5 | 0.3 | 0.7 |
| TITANIUM | 0.5 | 0.5 | 1.0 | 0.5 | 0.4 | 0.6 |
| DIOXIDE | | | | | | |
| TALC | 0.1 | 0.1 | 0.1 | 0.1 | 0.1 | 0.1 |
| WAX | 0.1 | 0.1 | 0.1 | 0.1 | 0.1 | 0.1 |
| GINGER | — | 0.33 | 1.0^{a)} | — | 0.33 | 1.0^{a)} |
| POWDER | | | | | | |
| or OIL | | | | | | |

^{a)}All of the active agent is in the coating, which comprises 33% of the product.

Maltitol powder is used to dry charge in the early stages of coating. Maltitol, gum arabic, and whitener are blended into a syrup and applied to pellets. After all coating is applied and dried, talc and wax are added to give a polish. Ginger powder or oil may be applied in a similar manner as in the previous xylitol coating examples, or preblended with the dry charge materials.

In a similar manner, coatings with sorbitol, lactitol, and hydrogenated isomaltulose may be made in the coating formulas in Table 52 by replacing maltitol with any one of the other polyols and maltitol powder with the polyol powder. Like maltitol, the other polyols may become sticky during the coating and drying process, so the dry powder charge may be needed to give the proper drying. In the later stages of the coating process, less gum arabic could be used and a more pure polyol syrup could be used to give a smooth surface. Also, the dry charge would only be used in the early stages of the coating process.

In addition to dry charging with the specific polyol, other ingredients may be added to the dry charge to help absorb moisture. These materials could be inert such as talc, calcium carbonate, magnesium carbonate, starches, gums like arabinogalactan, gum talha, gum arabic or other moisture absorbing materials. Also, powdered sweeteners or flavors could be added with the dry charge.

Some polyols such as sorbitol, maltitol, erythritol, lactitol, or hydrogenated isomaltulose are not sufficiently sweet compared to sugar or xylitol, so high intensity sweeteners may be added to the coating, such as aspartame, acesulfame K, salts of acesulfame, cyclamate and its salts, saccharin and its salts, alitame, sucralose, thaumatin, monellin, dihydrochalcone, glycyrrhizin, neotame, and combinations thereof. If a hot syrup is applied, heat may degrade the sweetener so only stable sweeteners should be used. Generally high intensity sweeteners are added with the polyol/gum arabic solution to obtain an even distribution in the coatings.

Liquid flavors generally are not added throughout the coating but at specific points throughout the process. When flavor is added, less air is used for drying until the flavor coating is covered by the next coatings and dried. Flavors may be various spearmint, peppermint, wintergreen, cinnamon, and fruit flavors to yield a wide variety of flavored chewing gum products.

As noted earlier, the gum formulas can be prepared as stick or tab products in the sugar or sugarless type formulations. These formulas can also be made in a pellet or pillow shape pellet or a round ball or any other shape of product for coating/panning. However, gum formulas are generally adjusted to a higher level of gum base to give a more consumer acceptable size of gum bolus.

As a result, gum centers are usually formulated with about 25% to about 40% gum base with a corresponding decrease in the other ingredients except flavor. Generally flavors increase with the level of gum base as the base tends to bind flavors into the gum and more flavor is needed to give a good flavorful product. However flavors can also be added to the coating to give increased flavor impact and more flavor perception.

Some typical sugar type gum center formulations are shown in Table 53, in which chuang lian extract (denoted chuang lian, or coptis chinensis) can be added as the active medicament. Chuang lian is an antibacterial effective against oral bacteria responsible for caries and halitosis. These formulas give a 1.5 gram piece containing 12.5 mg of chuang lian or 0.83% of the total gum product. Gum center formulas may or may not contain encapsulated or controlled release chuang lian.

TABLE 53
(WEIGHT PERCENT)

| | EX. 374 | EX. 375 | EX. 376 | EX. 377 | EX. 378 | EX. 379 |
|---|---|---|---|---|---|---|
| SUGAR | 52.0 | 48.17 | 46.75 | 44.0 | 40.17 | 37.75 |
| GUM BASE | 26.0 | 30.0 | 35.0 | 26.0 | 30.0 | 35.0 |
| CORN SYRUP | 20.0 | 19.0 | 15.0 | 18.0 | 17.0 | 14.0 |
| GLYCERIN | 1.0 | 1.0 | 1.0 | 1.0 | 1.0 | 1.0 |
| PEPPERMINT | 1.0 | 1.0 | 1.0 | 1.0 | 1.0 | 1.0 |
| FLAVOR | | | | | | |
| DEXTROSE | — | — | — | 10.0 | 10.0 | 10.0 |
| MONO- | | | | | | |
| HYDRATE | | | | | | |
| CHAUNG | —.^{a)} | 0.83 | 1.25 | —.^{a)} | 0.83 | 1.25 |
| LIAN | | | | | | |

^{a)}All of the active agent is in the coating, which comprises 33% of the product

Formulations with or without chuang lian can also be made similar to those found in previous tables for low, medium, and high moisture formulas. Higher levels of base may be used with a corresponding decrease in other ingredients. Also, other sugars and polyols may be used in the gum center as found in previous tables chuang lian may be added to a gum center only, into a gum coating with none in the center, or to both center and coating.

Chuang lian can be used in the coating formula on the various pellet gum formulations. The following Table 54 shows some sugar and dextrose type formulas:

TABLE 54
(DRY WEIGHT PERCENT)

| | EX. 380 | EX. 381 | EX. 382 | EX. 383 | EX. 384 | EX. 385 |
|---|---|---|---|---|---|---|
| SUGAR | 97.1 | 94.57 | 91.6 | 96.9 | 94.27 | 91.1 |
| GUM ARABIC | 2.0 | 3.0 | 4.0 | 2.0 | 3.0 | 4.0 |
| TITANIUM | 0.5 | 1.0 | 1.0 | — | — | — |
| DIOXIDE | | | | | | |
| CALCIUM | — | — | — | 0.5 | 1.0 | 2.0 |
| CARBONATE | | | | | | |
| FLAVOR | 0.3 | 0.5 | 0.8 | 0.5 | 0.8 | 0.3 |
| WAX | 0.1 | 0.1 | 0.1 | 0.1 | 0.1 | 0.1 |
| CHUANG | — | 0.83 | 2.5^{a)} | — | 0.83 | 2.5^{(a} |
| LIAN | | | | | | |

| | EX. 386 | EX. 387 | EX. 388 | EX. 389 |
|---|---|---|---|---|
| DEXTROSE | 97.6 | 94.56 | 96.37 | 92.0 |
| MONOHYDRATE | | | | |
| GUM ARABIC | 1.5 | 3.0 | 1.5 | 3.0 |
| TITANIUM DIOXIDE | 0.5 | 1.0 | — | — |
| CALCIUM CARBONATE | — | — | 1.0 | 2.0 |
| COOLING AGENT | — | 0.1 | — | 0.1 |
| FLAVOR | 0.3 | 0.5 | 0.2 | 0.3 |
| WAX | 0.1 | 0.1 | 0.1 | 0.1 |
| CHUANG LIAN | — | 0.83 | 0.83 | 2.5^{a)} |

^{a)}All of the active agent is in the coating, which comprises 33% of the product.

The above formulations are made by making a syrup by dissolving the sugar and gum arabic in solution at about 75% solids at boiling, and suspending titanium dioxide or calcium carbonate in this syrup. Chuang Lian may be dissolved in water, not mixed with hot syrup, but applied between coatings, or it may be added to the hot syrup and used in the early stages of coating or used throughout the coating process. Flavor is not mixed with the hot syrup, but added at low levels with one or more coats. Chuang Lian may also be premixed with the flavor. After the final coats are applied and dried, wax is applied to give a smooth polish.

The above process gives a hard shell coating. Often a dry charge of powdered sugar or dextrose monohydrate may be used. This gives a somewhat softer coating. A dry charge, which also may contain the active, may be used to build up a coating, but then finished with a straight syrup to obtain a hard shell. Table 55 gives these types of formulas.

TABLE 55
(DRY WEIGHT PERCENT)

| | EX. 390 | EX. 391 | EX. 392 | EX. 393 | EX. 394 | EX. 395 |
|---|---|---|---|---|---|---|
| SUGAR | 76.67 | 78.9 | — | — | 86.67 | — |
| DEXTROSE | — | — | 76.67 | 83.8 | — | 84.6 |
| MONO- | | | | | | |
| HYDRATE | | | | | | |
| POWDER | 20.0 | 15.0 | — | — | — | — |
| SUGAR* | | | | | | |
| POWDER | — | — | 20.0 | 10.0 | — | — |
| DEXTROSE* | | | | | | |
| GUM ARABIC | 2.0 | 3.0 | 2.0 | 3.0 | 8.0 | 8.0 |
| POWDER | | | | | | |
| GUM ARABIC | — | — | — | — | 4.0 | 4.0 |
| SOLUTION | | | | | | |
| FLAVOR | 0.4 | 0.5 | 0.4 | 0.6 | 0.4 | 0.8 |
| WAX | 0.1 | 0.1 | 0.1 | 0.1 | 0.1 | 0.1 |
| CHUANG | 0.83 | 2.5^{a)} | 0.83 | 2.5^{a)} | 0.83 | 2.5^{a)} |
| LIAN | | | | | | |

*Powder and/or crystalline sugar may be used.
^{a)}All of the active agent is in the coating, which comprises 33% of the product.

In Examples 390-393, gum arabic is blended in the sugar syrup. In Examples 394 and 395, gum arabic powder is dry charged after gum arabic solution is applied in the first stages of coating, then this is followed by a hard shell coating of sugar solution or dextrose solution.

CHUANG LIAN may also be used in coating of sugarless gum centers. Like sugar gum centers, the base formulation can be increased in proportion to the amount of coating applied to the center. Formulations with and without CHUANG LIAN similar to those found in previous tables for low and high moisture gum can be used to make gum centers. Generally, the base level may be increased to 30-46% with the other ingredients proportionally reduced. Some typical gum formulas are in Table 56.

TABLE 56
(WEIGHT PERCENT)

| | EX. | EX. | EX. | EX. | EX. | EX. | EX. |
|---|---|---|---|---|---|---|---|
| | 396 | 397 | 398 | 399 | 400 | 401 | 402 |
| GUM BASE | 35.0 | 35.0 | 30.0 | 30.0 | 30.0 | 40.0 | 50.0 |
| CALCIUM | — | — | 5.0 | 10.0 | 15.0 | — | — |
| CARBON- | | | | | | | |
| ATE | | | | | | | |
| SORBITOL | 43.3 | 44.46 | 45.05 | 40.1 | 43.97 | 40.42 | 24.83 |
| MANNITOL | 10.0 | 10.0 | 5.0 | 10.0 | — | 8.0 | 10.0 |
| GLYCERIN | — | 8.0 | 2.0 | — | 8.0 | 2.0 | 2.0 |
| SORBITOL | 10.0 | — | 10.0 | 8.0 | — | 6.0^{a)} | 10.0^{a)} |
| LIQUID | | | | | | | |
| COOLING | — | 0.1 | — | 0.2 | — | 0.3 | — |
| AGENT | | | | | | | |
| FLAVOR | 1.5 | 1.5 | 1.5 | 1.5 | 2.0 | 2.0 | 1.3 |
| HIGH- | 0.2 | 0.2 | 0.2 | 0.2 | 0.2 | 0.3 | 0.2 |
| INTENSITY | | | | | | | |
| SWEET- | | | | | | | |
| ENER | | | | | | | |
| CHUANG | —^{c)} | 0.83 | 1.25 | —^{c)} | 0.83 | 1.25 | 1.67^{d)} |
| LIAN^{b)} | | | | | | | |

^{a)}Lycasin brand hydrogenated starch hydrolyzate used instead of sorbitol liquid.
^{b)}CHUANG LIAN may be dissolved/suspended in water, glycerin, sorbitol liquid, HSH, or flavor.
^{c)}All of the active agent is in the coating, which comprises 33% of the product.
^{d)}This example requires 50% of the product to be a coating with no active agent in the coating, to give a gum product with 0.83% active agent.

In the above center formulations, the high intensity sweetener used is aspartame. However other high intensity such as alitame, acesulfame K, salts of acesulfame, cyclamate and its salts, saccharin and its salts, neotame, sucralose, thaumatin, monellin, dihydrochalcone, stevioside, glycyrrhizin and combinations thereof may be used in any of the examples with the level adjusted for sweetness.

Lycasin and other polyols such as maltitol, xylitol, erythritol, lactitol and hydrogenated isomaltulose may also be used in the gum center formulations at various levels. The texture may be adjusted by varying glycerin or sorbitol liquid. Sweetness of the center formulation can also be adjusted by varying the level of high intensity sweetener.

Chuang Lian may be used in sugarless coatings with xylitol, sorbitol, maltitol, lactitol, hydrogenated isomaltulose and erythritol. The following table gives formulas for a xylitol coating:

TABLE 57
(DRY WEIGHT PERCENT)

| | EX. | EX. | EX. | EX. | EX. | EX. |
|---|---|---|---|---|---|---|
| | 403 | 404 | 405 | 406 | 407 | 408 |
| XYLITOL | 94.8 | 91.57 | 88.2 | 90.1 | 89.07 | 86.3 |
| GUM ARABIC | 4.0 | 6.0 | 7.0 | 8.5 | 8.5 | 10.0 |
| FLAVOR | 0.5 | 0.5 | 0.7 | 0.7 | 0.9 | 0.5 |
| TITANIUM | 0.5 | 0.9 | — | 0.5 | 0.5** | 0.5** |
| DIOXIDE | | | | | | |
| TALC | 0.1 | 0.1 | 0.1 | 0.1 | 0.1 | 0.1 |
| WAX | 0.1 | 0.1 | 0.1 | 0.1 | 0.1 | 0.1 |
| COLOR* | — | — | 1.4 | — | — | — |
| CHUANG LIAN | — | 0.83 | 2.5^{a)} | — | 0.83 | 2.5^{a)} |

*Lake color dispersed in xylitol solution.
**Calcium carbonate used in place of titanium dioxide.
^{a)}All of the active agent is in the coating, which comprises 33% of the product.

The above formulas are used to coat pellets by applying a xylitol/gum arabic solution in multiple coats and air drying. Color or whitener is also mixed in the solution. After pellets have been coated and dried, talc and wax are added to give a polish. Chuang lian may be dissolved in water or flavor and added between coating applications, or mixed with the hot syrup and used in the early stages of coating or used throughout the coating process.

For coating formulas based on sorbitol, maltitol, lactitol, erythritol, and hydrogenated isomaltulose, gum arabic can be used as a binder and film former, and a crystallization modifier to help facilitate coating. Generally these polyols are more difficult to coat using only a straight syrup, but with proper technique a good smooth hard shell can be made. However, it may be preferable to add a dry charge to quicken the drying process before the pellets get too sticky. The following formulations may be used.

TABLE 58
(DRY WEIGHT PERCENT)

| | EX. | EX. | EX. | EX. | EX. | EX. |
|---|---|---|---|---|---|---|
| | 409 | 410 | 411 | 412 | 413 | 414 |
| MALTITOL | 96.8 | 94.07 | 89.6 | 86.8 | 75.27 | 67.0 |
| MALTITOL | — | — | — | 10.0 | 20.0 | 25.0 |
| POWDER | | | | | | |
| GUM ARABIC | 2.0 | 4.0 | 6.0 | 2.0 | 3.0 | 4.0 |
| FLAVOR | 0.5 | 0.4 | 0.7 | 0.5 | 0.3 | 0.7 |
| TITANIUM | 0.5 | 0.5 | 1.0 | 0.5 | 0.4 | 0.6 |
| DIOXIDE | | | | | | |
| TALC | 0.1 | 0.1 | 0.1 | 0.1 | 0.1 | 0.1 |
| WAX | 0.1 | 0.1 | 0.1 | 0.1 | 0.1 | 0.1 |
| CHUANG | — | 0.83 | 2.5^{a)} | — | 0.83 | 2.5^{a)} |
| LIAN | | | | | | |

^{a)}All of the active agent is in the coating, which comprises 33% of the product.

Maltitol powder is used to dry charge in the early stages of coating. Maltitol, gum arabic, and whitener are blended into a syrup and applied to pellets. After all coating is applied and dried, talc and wax are added to give a polish. Chuang lian may be applied in a similar manner as in the previous xylitol coating examples, or preblended with the dry charge material and added to the coating.

In a similar manner, coatings with sorbitol, lactitol, and hydrogenated isomaltulose may be made in the coating formulas in Table 58 by replacing maltitol with any one of the other polyols and maltitol powder with the polyol powder. Like maltitol, the other polyols may become sticky during the coating and drying process, so the dry powder charge may be needed to give the proper drying. In the later stages of the coating process, less gum arabic could be used and a more pure polyol syrup could be used to give a smooth surface. Also, the dry charge would only be used in the early stages of the coating process.

In addition to dry charging with the specific polyol, other ingredients may be added to the dry charge to help absorb moisture. These materials could be inert such as talc, calcium carbonate, magnesium carbonate, starches, gums like arabinogalactan, gum talha, gum arabic or other moisture absorbing materials. Also, powdered sweeteners or flavors could be added with the dry charge.

Some polyols such as sorbitol, maltitol, erythritol, lactitol, or hydrogenated isomaltulose are not sufficiently sweet compared to sugar or xylitol, so high intensity sweeteners may be added to the coating, such as aspartame, acesulfame K, salts of acesulfame, cyclamate and its salts, saccharin and its salts, alitame, sucralose, thaumatin, monellin, dihydrochalcone, glycyrrhizin, neotame, and combinations thereof. If a hot syrup is applied, heat may degrade the sweetener so only stable sweeteners should be used. Generally high intensity sweeteners are added with the polyol/gum arabic solution to obtain an even distribution in the coatings.

As noted earlier, the gum formulas can be prepared as stick or tab products in the sugar or sugarless type formulations. These formulas can also be made in a pellet or pillow shape pellet or a round ball or any other shape of product for coating/panning. However, gum formulas are generally adjusted to a higher level of gum base to give a more consumer acceptable size of gum bolus.

As a result, gum centers are usually formulated with about 25% to about 40% gum base with a corresponding decrease in the other ingredients except flavor. Generally flavors increase with the level of gum base as the base tends to bind flavors into the gum and more flavor is needed to give a good flavorful product. However flavors can also be added to the coating to give increased flavor impact and more flavor perception.

Some typical sugar type gum center formulations are shown in Table 59 in which lithospermum erythrorhizon extract (denoted L. Erythrorhizon) can be added as the active medicament. This material is an antibacterial effective against organisms responsible for bad breath and cavities. These formulas give a 1.5 gram piece containing 15 mg of L. Erythrorhizon or 1.0% of gum product. Gum centers may or may not contain L. Erythrorhizon.

TABLE 59
(WEIGHT PERCENT)

| | EX. | EX. | EX. | EX. | EX. | EX. |
|---|---|---|---|---|---|---|
| | 415 | 416 | 417 | 418 | 419 | 420 |
| SUGAR | 52.0 | 48.0 | 46.5 | 44.0 | 40.0 | 37.5 |
| GUM BASE | 26.0 | 30.0 | 35.0 | 26.0 | 30.0 | 35.0 |
| CORN SYRUP | 20.0 | 19.0 | 15.0 | 18.0 | 17.0 | 14.0 |
| GLYCERIN | 1.0 | 1.0 | 1.0 | 1.0 | 1.0 | 1.0 |
| PEPPERMINT | 1.0 | 1.0 | 1.0 | 1.0 | 1.0 | 1.0 |
| FLAVOR | | | | | | |
| DEXTROSE | — | — | — | 10.0 | 10.0 | 10.0 |
| MONOHYDRATE | | | | | | |
| L. Erythrorhizon | —^{a)} | 1.0 | 1.5 | —^{a)} | 1.0 | 1.5 |

^{a)}All of the active agent is in the coating, which comprises 33% of the product.

Formulations with or without L. Erythrorhizon can also be made similar to those found previously in Tables for low, medium, and high moisture formulas. Higher levels of base may be used with a corresponding decrease in other ingredients. Also, other sugars and polyols may be used in the gum center as found in previous tables. L. Erythrorhizon may be added to the gum center only, into a gum coating with none in the center, or both center and coating.

L. Erythrorhizon can then be used in the coating formula on the various pellet gum formulations. The following Table 60 shows some sugar and dextrose type formulas:

TABLE 60
(DRY WEIGHT PERCENT)

| | EX. | EX. | EX. | EX. | EX. | EX. |
|---|---|---|---|---|---|---|
| | 421 | 422 | 423 | 424 | 425 | 426 |
| SUGAR | 97.1 | 94.4 | 91.1 | 96.9 | 94.1 | 90.6 |
| GUM ARABIC | 2.0 | 3.0 | 4.0 | 2.0 | 3.0 | 4.0 |
| TITANIUM | 0.5 | 1.0 | 1.0 | — | — | — |
| DIOXIDE | | | | | | |
| CALCIUM | — | — | — | 0.5 | 1.0 | 2.0 |
| CARBONATE | | | | | | |
| FLAVOR | 0.3 | 0.5 | 0.8 | 0.5 | 0.8 | 0.3 |
| WAX | 0.1 | 0.1 | 0.1 | 0.1 | 0.1 | 0.1 |
| L. Erythrorhizon | — | 1.0 | 3.0^{a)} | — | 1.0 | 3.0^{a)} |

| | EX. 427 | EX. 428 | EX. 429 | EX. 430 |
|---|---|---|---|---|
| DEXTROSE | 97.6 | 94.4 | 96.2 | 91.5 |
| MONOHYDRATE | | | | |
| GUM ARABIC | 1.5 | 3.0 | 1.5 | 3.0 |
| TITANIUM DIOXIDE | 0.5 | 1.0 | — | — |
| CALCIUM CARBONATE | — | — | 1.0 | 2.0 |
| FLAVOR | 0.3 | 0.5 | 0.2 | 0.4 |
| WAX | 0.1 | 0.1 | 0.1 | 0.1 |
| L. Erythrorhizon | — | 1.0 | 1.0 | 3.0^{a)} |

^{a)}All of the active agent is in the coating, which comprises 33% of the product.

The above formulations are made by making a syrup by dissolving the sugar and gum arabic in solution at about 75% solids at boiling, and suspending titanium dioxide or calcium carbonate in this syrup. L. Erythrorhizon may be dissolved in water, not mixed with hot syrup, but applied between coatings, or it may be added to the hot syrup and used in the early stages of coating or used throughout the coating process. Flavor is not mixed with the hot syrup, but added at low levels with one or more coats. L. Erythrorhizon may also be premixed with the flavor. After the final coats are applied and dried, wax is applied to give a smooth polish.

The above process gives a hard shell coating. Often a dry charge of powdered sugar or dextrose monohydrate may be used. This gives a somewhat softer coating. A dry charge may be used to build up a coating, but then finished with a straight syrup to obtain a hard shell. L. Erythrorhizon may also be added to the dry charge material. Table 61 gives these types of formulas.

TABLE 61
(DRY WEIGHT PERCENT)

| | EX. | EX. | EX. | EX. | EX. | EX. |
|---|---|---|---|---|---|---|
| | 431 | 432 | 433 | 434 | 435 | 436 |
| SUGAR | 76.5 | 78.4 | — | — | 86.5 | — |
| DEXTROSE | — | — | 76.5 | 83.3 | — | 84.1 |
| MONO- | | | | | | |
| HYDRATE | | | | | | |
| POWDER | 20.0 | 15.0 | — | — | — | — |
| SUGAR* | | | | | | |
| POWDER | — | — | 20.0 | 10.0 | — | — |
| DEXTROSE* | | | | | | |
| GUM ARABIC | 2.0 | 3.0 | 2.0 | 3.0 | 8.0 | 8.0 |
| POWDER | | | | | | |
| GUM ARABIC | — | — | — | — | 4.0 | 4.0 |
| SOLUTION | | | | | | |
| FLAVOR | 0.4 | 0.5 | 0.4 | 0.6 | 0.4 | 0.8 |
| WAX | 0.1 | 0.1 | 0.1 | 0.1 | 0.1 | 0.1 |
| L. Erythrorhizon | 1.0 | 3.0^{a)} | 1.0 | 3.0^{a)} | 1.0 | 3.0^{a)} |

*Powder and/or crystalline sugar may be used.
^{a)}All of the active agent is in the coating, which comprises 33% of the product.

In Examples 431-434 gum arabic is blended in the sugar syrup. In Examples 435 and 436, gum arabic powder is dry charged after a gum arabic solution is applied in the first stages of coating, then this is followed by a hard shell coating of sugar solution or dextrose.

L. Erythrorhizon may also be used in coating of sugarless gum centers. Like sugar gum centers, the base formulation can be increased in proportion to the amount of coating applied to the center. Formulations with and without L. Erythrorhizon similar to those found in previous tables for low and high moisture gum can be used to make gum centers. Generally, the base level may be increased to 30-46% with the other ingredients proportionally reduced. Some typical gum center formulas are in Table 62.

TABLE 62
(WEIGHT PERCENT)

| | EX. | EX. | EX. | EX. | EX. | EX. | EX. |
|---|---|---|---|---|---|---|---|
| | 437 | 438 | 439 | 440 | 441 | 442 | 443 |
| GUM BASE | 35.0 | 35.0 | 30.0 | 30.0 | 30.0 | 40.0 | 50.0 |
| CALCIUM | — | — | 5.0 | 10.0 | 15.0 | — | — |
| CARBONATE | | | | | | | |
| SORBITOL | 43.2 | 44.2 | 43.3 | 40.3 | 43.8 | 38.7 | 24.5 |
| MANNITOL | 10.0 | 10.0 | 5.0 | 10.0 | — | 8.0 | 10.0 |
| GLYCERIN | — | 8.0 | 2.0 | — | 8.0 | 2.0 | 2.0 |
| SORBITOL | 10.0 | — | 10.0 | 8.0 | — | 6.0^{a)} | 10.0^{a)} |
| LIQUID | | | | | | | |
| COOLING | 0.1 | 0.1 | — | — | — | — | — |
| AGENT | | | | | | | |
| FLAVOR | 1.5 | 1.5 | 1.5 | 1.5 | 2.0 | 2.0 | 1.3 |
| HIGH- | 0.2 | 0.2 | 0.2 | 0.2 | 0.2 | 0.3 | 0.2 |
| INTENSITY | | | | | | | |
| SWEETENER | | | | | | | |
| L. | —^{c)} | 1.0 | 3.0 | —^{c)} | 1.0 | 3.0 | 2.0^{d)} |
| Erythrorhizon^{b)} | | | | | | | |

^{a)}Lycasin brand hydrogenated starch hydrolyzate used instead of sorbitol liquid
^{b)}L. Erythrorhizon may be dissolved/suspended in water, glycerin, sorbitol liquid, HSH, or flavor
^{c)}All of the active agent is in the coating, which comprises 33% of the product
^{d)}This example requires 50% of the product to be a coating with no active agent in the coating, to give a gum product with 1% active agent.

In the above center formulations, the high intensity sweetener used is aspartame. However other high intensity such as alitame, acesulfame K, salts of acesulfame, cyclamate and its salts, saccharin and its salts, neotame, sucralose, thaumatin, monellin, dihydrochalcone, stevioside, glycyrrhizin and combinations thereof may be used in any of the examples with the level adjusted for sweetness.

Lycasin and other polyols such as maltitol, xylitol, erythritol, lactitol and hydrogenated isomaltulose may also be used in the gum center formulations at various levels. The texture may be adjusted by varying glycerin or sorbitol liquid. Sweetness of the center formulation can also be adjusted by varying the level of high intensity sweetener.

L. Erythrorhizon may be used in sugarless coatings with xylitol, sorbitol, maltitol, lactitol, hydrogenated isomaltulose and erythritol. The following table gives formulas for a xylitol coating:

TABLE 63
(DRY WEIGHT PERCENT)

| | EX. | EX. | EX. | EX. | EX. | EX. |
|---|---|---|---|---|---|---|
| | 444 | 445 | 446 | 447 | 448 | 449 |
| XYLITOL | 94.8 | 91.4 | 87.7 | 90.1 | 88.9 | 85.8 |
| GUM ARABIC | 4.0 | 6.0 | 7.0 | 8.5 | 8.5 | 10.0 |
| FLAVOR | 0.5 | 0.5 | 0.7 | 0.7 | 0.9 | 0.5 |
| TITANIUM DIOXIDE | 0.5 | 0.9 | — | 0.5 | 0.5** | 0.5** |
| TALC | 0.1 | 0.1 | 0.1 | 0.1 | 0.1 | 0.1 |
| WAX | 0.1 | 0.1 | 0.1 | 0.1 | 0.1 | 0.1 |
| COLOR* | — | — | 1.4 | — | — | — |
| L. ERYTHRORHIZON | — | 1.0 | 3.0^{a)} | — | 1.0 | 3.0^{a)} |

*Lake color dispersed in xylitol solution
**Calcium carbonate used in place of titanium dioxide
^{a)}All of the active agent is in the coating, which comprises 33% of the product.

The above formulas are used to coat pellets by applying a xylitol/gum arabic solution in multiple coats and air drying. Color or whitener is also mixed in the solution. After pellets have been coated and dried, talc and wax are added to give a polish. L. Erythrorhizon may be dissolved/suspended in water or flavor and added between coating applications, or mixed with hot syrup and used in the early stages of coating or used throughout the coating process.

For coating formulas based on sorbitol, maltitol, lactitol, erythritol, and hydrogenated isomaltulose, gum arabic can be used as a binder and film former, and a crystallization modifier to help facilitate coating. Generally these polyols are more difficult to coat using only a straight syrup, but with proper technique a good smooth hard shell can be made. However, it may be preferable to add a dry charge to quicken the drying process before the pellets get too sticky. The active may be premixed with the dry charge material. The following formulations may be used.

TABLE 64
(DRY WEIGHT PERCENT)

| | EX. | EX. | EX. | EX. | EX. | EX. |
|---|---|---|---|---|---|---|
| | 450 | 451 | 452 | 453 | 454 | 455 |
| MALTITOL | 96.8 | 93.9 | 89.1 | 91.8 | 85.1 | 76.5 |
| MALTITOL POWDER | — | — | — | 5.0 | 10.0 | 15.0 |
| GUM ARABIC | 2.0 | 4.0 | 6.0 | 2.0 | 3.0 | 4.0 |
| FLAVOR | 0.5 | 0.4 | 0.7 | 0.5 | 0.3 | 0.7 |
| TITANIUM DIOXIDE | 0.5 | 0.5 | 1.0 | 0.5 | 0.4 | 0.6 |
| TALC | 0.1 | 0.1 | 0.1 | 0.1 | 0.1 | 0.1 |
| WAX | 0.1 | 0.1 | 0.1 | 0.1 | 0.1 | 0.1 |
| L. ERYTHRORHIZON | — | 1.0 | 3.0^{a}) | — | 1.0 | 3.0^{a}) |

^{a})All of the active agent is in the coating, which comprises 33% of the product.

Maltitol powder is used to dry charge in the early stages of coating. Maltitol, gum arabic, and whitener is blended into a syrup and applied to pellets. After all coating is applied and dried, talc and wax are added to give a polish. L. Erythrorhizon may be applied in a similar manner as the previous xylitol examples, or added with the dry charge material.

In a similar manner, coatings with sorbitol, lactitol, and hydrogenated isomaltulose may be made in the coating formulas in Table 64 by replacing maltitol with any one of the other polyols and maltitol powder with the polyol powder. Like maltitol, the other polyols may become sticky during the coating and drying process, so the dry powder charge may be needed to give the proper drying. In the later stages of the coating process, less gum arabic could be used and a more pure polyol syrup could be used to give a smooth surface. Also, the dry charge would only be used in the early stages of the coating process.

In addition to dry charging with the specific polyol, other ingredients may be added to the dry charge to help absorb moisture. These materials could be inert such as talc, calcium carbonate, magnesium carbonate, starches, gums like arabinogalactan, gum talha, gum arabic or other moisture absorbing materials. Also, powdered sweeteners or flavors could be added with the dry charge.

Some polyols such as sorbitol, maltitol, erythritol, lactitol, or hydrogenated isomaltulose are not sufficiently sweet compared to sugar or xylitol, so high intensity sweeteners may be added to the coating, such as aspartame, acesulfame K, salts of acesulfame, cyclamate and its salts, saccharin and its salts, alitame, sucralose, thaumatin, monellin, dihydrochalcone, glycyrrhizin, neotame, and combinations thereof. If a hot syrup is applied, heat may degrade the sweetener so only stable sweeteners should be used. Generally high intensity sweeteners are added with the polyol/gum arabic solution to obtain an even distribution in the coatings.

As noted earlier, the gum formulas can be prepared as stick or tab products in the sugar or sugarless type formulations. These formulas can also be made in a pellet or pillow shape pellet or a round ball or any other shape of product for coating/panning. However, gum formulas are generally adjusted to a higher level of gum base to give a more consumer acceptable size of gum bolus.

As a result, gum centers are usually formulated with about 25% to about 40% gum base with a corresponding decrease in the other ingredients except flavor. Generally flavors increase with the level of gum base as the base tends to bind flavors into the gum and more flavor is needed to give a good flavorful product. However flavors can also be added to the coating to give increased flavor impact and more flavor perception.

Some typical sugar type gum center formulations are shown in Table 65 that can be used as centers that are coated with calcium carbonate to give an effective antacid.

TABLE 65
(WEIGHT PERCENT)

| | | | | | EX. | EX. |
|---|---|---|---|---|---|---|
| | EX. 456 | EX. 457 | EX. 458 | EX. 459 | 460 | 461 |
| SUGAR | 48.0 | 48.0 | 46.0 | 40.0 | 39.0 | 36.0 |
| GUM BASE | 30.0 | 35.0 | 40.0 | 30.0 | 35.0 | 40.0 |
| CORN SYRUP | 20.0 | 15.0 | 12.0 | 18.0 | 14.0 | 12.0 |
| GLYCERIN | 1.0 | 1.0 | 1.0 | 1.0 | 1.0 | 1.0 |
| FRUIT FLAVOR | — | — | 1.0 | — | 0.2 | — |
| PEPPERMINT | 1.0 | 1.0 | — | 1.0 | 0.8 | 1.0 |
| FLAVOR | | | | | | |
| DEXTROSE | — | — | — | 10.0 | 10.0 | 10.0 |
| MONOHYDRATE | | | | | | |

Formulations can also be made similar to those found in previous tables for low, medium, and high moisture formulas. Higher levels of base may be used with a corresponding decrease in other ingredients. Also, other sugars may be used in the gum center as found in previous tables.

Bloat Fruit seeds ground or there extracts (Denoted Bloat in the examples) can then be used in the coating formula on the various pellet gum formulations. Bloat relieves coughing and reduces internal heat, and throat irritation. The following Table 66 shows some sugar and dextrose type formulas: Using a 1 gram center, the levels of Bloat in the following tables will give 250-800 mg per 2 pieces in 1.5-3.0 gum pieces with 33 to 50% coating.

TABLE 66
(DRY WEIGHT PERCENT)

| | | EX. | EX. | EX. | EX. | EX. |
|---|---|---|---|---|---|---|
| | EX. 462 | 463 | 464 | 465 | 466 | 467 |
| SUGAR | 72.1 | 65.4 | 54.1 | 72.4 | 66.1 | 55.6 |
| GUM ARABIC | 2.0 | 3.0 | 4.0 | 2.0 | 3.0 | 4.0 |
| TITANIUM DIOXIDE | 0.5 | 1.0 | 1.0 | — | — | — |
| Bloat | 25.0 | 30.0 | 40.0 | 25.0 | 30.0 | 40.0 |
| FLAVOR | 0.3 | 0.5 | 0.8 | 0.5 | 0.8 | 0.3 |
| WAX | 0.1 | 0.1 | 0.1 | 0.1 | 0.1 | 0.1 |

| | EX. 468 | EX. 469 | EX. 470 | EX. 471 |
|---|---|---|---|---|
| DEXTROSE MONOHYDRATE | 72.6 | 55.4 | 73.2 | 56.5 |
| GUM ARABIC | 1.5 | 3.0 | 1.5 | 3.0 |
| TITANIUM DIOXIDE | 0.5 | 1.0 | — | — |
| Bloat | 25.0 | 40.0 | 25.0 | 40.0 |
| FLAVOR | 0.3 | 0.5 | 0.2 | 0.4 |
| WAX | 0.1 | 0.1 | 0.1 | 0.1 |

The above formulations are made by making a syrup by dissolving the sugar and gum arabic in solution at about 75% solids at boiling, and suspending titanium dioxide and/or calcium carbonate in this syrup. Flavor is not mixed with the hot syrup, but added at low levels with one or more coats. After the final coats are applied and dried, wax is applied to give a smooth polish.

The above process gives a hard shell coating. Often a dry charge of powdered sugar or dextrose monohydrate may be used. This gives a somewhat softer coating. A dry charge may be used to build up a coating, but then finished with a straight syrup to obtain a hard shell. Table 67 gives these types of formulas.

TABLE 67
(DRY WEIGHT PERCENT)

| | EX. | EX. | EX. | EX. | EX. | EX. |
|---|---|---|---|---|---|---|
| | 472 | 473 | 474 | 475 | 476 | 477 |
| SUGAR | 62.5 | 51.4 | — | — | 52.5 | — |
| DEXTROSE | — | — | 62.5 | 51.3 | — | 42.1 |
| MONOHYDRATE | | | | | | |
| POWDER SUGAR* | 10.0 | 5.0 | — | — | — | — |
| POWDER DEXTROSE* | — | — | 10.0 | 5.0 | 10.0 | 5.0 |
| GUM ARABIC POWDER* | 2.0 | 3.0 | 2.0 | 3.0 | 8.0 | 8.0 |
| GUM ARABIC SOLUTION | — | — | — | — | 4.0 | 4.0 |
| FLAVOR | 0.4 | 0.5 | 0.4 | 0.6 | 0.4 | 0.8 |
| WAX | 0.1 | 0.1 | 0.1 | 0.1 | 0.1 | 0.1 |
| BLOAT | 25.0 | 40.0 | 25.0 | 40.0 | 25.0 | 40.0 |

*Powder and/or crystalline sugar along with gum arabic may be blended with calcium carbonate, or calcium carbonate may be suspended in the sugar or dextrose syrup.

In Examples 472-475, gum arabic is blended in the sugar syrup. In Examples 476 and 477, gum arabic powder is dry charged after a gum arabic solution is applied in the first stages of coating, then this is followed by a hard shell coating of sugar solution or dextrose solution.

Gum arabic may also be used in coating of sugarless gum centers. Like sugar gum centers, the base formulation can be increased in proportion to the amount of coating applied to the center. Formulations similar to those found in previous tables for low and high moisture gum can be used to make gum centers. Generally, the base level may be increased to 30-46% with the other ingredients proportionally reduced. Some typical gum formulas are in Table 68.

TABLE 68
(WEIGHT PERCENT)

| | EX. | EX. | EX. | EX. | EX. | EX. | EX. |
|---|---|---|---|---|---|---|---|
| | 478 | 479 | 480 | 481 | 482 | 483 | 484 |
| GUM BASE | 35.0 | 35.0 | 30.0 | 30.0 | 30.0 | 40.0 | 30.0 |
| Bloat | — | 1.0 | 5.0 | 10.0 | 10.0 | 20.0 | 35 |
| SORBITOL | 43.3 | 44.3 | 46.3 | 40.3 | 49.8 | 21.7 | 11.5 |
| MANNITOL | 10.0 | 10.0 | 5.0 | 10.0 | — | 8.0 | 10.0 |
| GLYCERIN | — | 8.0 | 2.0 | — | 8.0 | 2.0 | 2.0 |
| SORBITOL LIQUID | 10.0 | — | 10.0 | 8.0 | — | 6.0^{a)} | 10.0^{a)} |
| FLAVOR | 1.5 | 1.5 | 1.5 | 1.5 | 2.0 | 2.0 | 1.3 |
| HIGH-INTENSITY | 0.2 | 0.2 | 0.2 | 0.2 | 0.2 | 0.3 | 0.2 |
| SWEETENER | | | | | | | |

^{a)}Lycasin brand hydrogenated starch hydrolyzate used instead of sorbitol liquid

In the above center formulations, the high intensity sweetener used is aspartame. However other high intensity sweetener such as alitame, acesulfame K, salts of acesulfame, cyclamate and its salts, saccharin and its salts, neotame, sucralose, thaumatin, monellin, dihydrochalcone, stevioside, glycyrrhizin and combinations thereof may be used in any of the examples with the level adjusted for sweetness.

Lycasin and other polyols such as maltitol, xylitol, erythritol, lactitol and hydrogenated isomaltulose may also be used in the gum center formulations at various levels similar to those shown previously. The texture may be adjusted by varying glycerin or sorbitol liquid. Sweetness of the center formulation can also be adjusted by varying the level of high intensity sweetener.

Bloat can be used in sugarless coatings with xylitol, sorbitol, maltitol, lactitol, hydrogenated isomaltulose and erythritol. Gum arabic acts as a binder, film former, hardener of the coated pellet. The following table gives formulas for a xylitol coating:

TABLE 69
(DRY WEIGHT PERCENT)

| | | | | EX. | EX. | EX. |
|---|---|---|---|---|---|---|
| | EX. 485 | EX. 486 | EX. 487 | 488 | 489 | 490 |
| XYLITOL | 69.8 | 52.4 | 65.7 | 50.6 | 65.4 | 49.3 |
| GUM ARABIC | 4.0 | 6.0 | 7.0 | 8.5 | 8.5 | 10.0 |
| FLAVOR | 0.5 | 0.5 | 0.7 | 0.7 | 0.9 | 0.5 |
| TITANIUM DIOXIDE | 0.5 | 0.9 | — | — | — | — |
| TALC | 0.1 | 0.1 | 0.1 | 0.1 | 0.1 | 0.1 |
| WAX | 0.1 | 0.1 | 0.1 | 0.1 | 0.1 | 0.1 |
| COLOR* | — | — | 1.4 | — | — | — |
| BLOAT | 25.0 | 40.0 | 25.0 | 40.0 | 25.0 | 40.0 |

*Lake color dispersed in xylitol solution

The above formulas are used to coat pellets by applying a xylitol/gum arabic solution in multiple coats and air drying. Color or whitener is also mixed in the solution. Bloat may be suspended partially dissolved in the xylitol hot syrup or added as a dry powder between syrup applications. After pellets have been coated and dried, talc and wax are added to give a polish.

Like xylitol, erythritol coating also requires a binder, film former, and hardener in the coating to make an acceptable product. The following formulations can be made:

TABLE 70
(DRY WEIGHT PERCENT)

| | | | | EX. | EX. | EX. |
|---|---|---|---|---|---|---|
| | EX. 491 | EX. 492 | EX. 493 | 494 | 495 | 496 |
| ERYTHRITOL | 68.8 | 51.5 | 64.2 | 50.1 | 63.4 | 46.8 |
| GUM ARABIC | 5.0 | 7.0 | 8.5 | 8.5 | 10.0 | 12.0 |
| FLAVOR | 0.5 | 0.4 | 0.7 | 0.7 | 0.9 | 0.5 |
| TITANIUM DIOXIDE | 0.5 | 0.9 | — | 0.5 | 0.5 | 0.5 |
| TALC | 0.1 | 0.1 | 0.1 | 0.1 | 0.1 | 0.1 |
| WAX | 0.1 | 0.1 | 0.1 | 0.1 | 0.1 | 0.1 |
| COLOR | — | — | 1.4* | — | — | — |
| BLOAT | 25.0 | 40.0 | 25.0 | 40.0 | 25.0 | 40.0 |

*Lake color dispersed in erythritol solution

The above formulas are used to coat pellets by applying a erythritol/gum arabic solution in multiple coats and air drying. Color or whitener is also mixed in the solution. Bloat may be suspended in the hot erythritol syrup or added as a dry powder between syrup applications. After pellets have been coated and dried, talc and wax are added to give a polish.

For coating formulas based on sorbitol, maltitol, lactitol, and hydrogenated isomaltulose, gum arabic can be used as a binder and film former, and a crystallization modifier to help facilitate coating. Generally these polyols are more difficult to coat using only a straight syrup, but with proper technique a good smooth hard shell can be made. However, it may be preferable to add a dry charge to quicken the drying process before the pellets get too sticky. The following formulations may be used.

TABLE 71
(DRY WEIGHT PERCENT)

| | | | EX. | EX. | EX. | EX. |
|---|---|---|---|---|---|---|
| | EX. 497 | EX. 498 | 499 | 500 | 501 | 502 |
| MALTITOL | 71.8 | 54.9 | 67.1 | 51.8 | 61.1 | 39.5 |
| MALTITOL POWDER | — | — | — | 5.0 | 10.0 | 15.0 |
| GUM ARABIC | 2.0 | 4.0 | 6.0 | 2.0 | 3.0 | 4.0 |
| FLAVOR | 0.5 | 0.4 | 0.7 | 0.5 | 0.3 | 0.7 |
| TITANIUM DIOXIDE | 0.5 | 0.5 | 1.0 | 0.5 | 0.4 | 0.6 |
| TALC | 0.1 | 0.1 | 0.1 | 0.1 | 0.1 | 0.1 |
| WAX | 0.1 | 0.1 | 0.1 | 0.1 | 0.1 | 0.1 |
| BLOAT | 25.0 | 40.0 | 25.0 | 40.0 | 25.0 | 40.0 |

Maltitol powder is used to dry charge in the early stages of coating. Maltitol, gum arabic, and whitener is blended into a syrup and applied to pellets. Bloat may be applied with the syrup suspension, preblended with powder maltitol or added as a dry charge. After all coating is applied and dried, talc and wax are added to give a polish.

In a similar manner, coatings with sorbitol, lactitol, and hydrogenated isomaltulose may be made in the coating formulas in Table 71 by replacing maltitol with any one of the other polyols and maltitol powder with the polyol powder. Like maltitol, the other polyols may become sticky during the coating and drying process, so the dry powder charge may be needed to give the proper drying. In the later stages of the coating process, less gum arabic could be used and a more pure polyol syrup could be used to give a smooth surface. Also, the dry charge would only be used in the early stages of the coating process.

In addition to dry charging with the specific polyol, other ingredients may be added to the dry charge to help absorb moisture. These materials could be inert such as talc, magnesium carbonate, starches, gums like arabinogalactan, gum talha, gum arabic or other moisture absorbing materials. Also, powdered sweeteners or flavors could be added with the dry charge.

Some polyols such as sorbitol, maltitol, lactitol, or hydrogenated isomaltulose are not sufficiently sweet compared to sugar or xylitol, so high intensity sweeteners may be added to the coating, such as aspartame, acesulfame K, salts of acesulfame, cyclamate and its salts, saccharin and its salts, alitame, sucralose, thaumatin, monellin, dihydrochalcone, glycyrrhizin, neotame, and combinations thereof. If a hot syrup is applied, heat may degrade the sweetener so only stable sweeteners should be used. Generally high intensity sweeteners are added with the polyol/gum arabic solution to obtain an even distribution in the coatings.

Liquid flavors generally are not added throughout the coating but at specific points throughout the process. When flavor is added, less air is used for drying until the flavor coating is covered by the next coatings and dried. Flavors may be various spearmint, peppermint, wintergreen, cinnamon, and fruit flavors to yield a wide variety of flavored chewing gum products.

### Candy Examples

TABLE 72
(DRY WEIGHT PERCENT)

| | EX. 503 | EX. 504 | EX. 505 | EX. 506 | EX. 507 |
|---|---|---|---|---|---|
| CORN SYRUP | 44.0 | — | 44.0 | 47.3 | — |
| SUGAR | 53.5 | — | 51.0 | 47.0 | — |
| POLYALCOHOL | — | 95.3 | — | — | 95.6 |
| FLAVOR | 1.0 | 3.0 | 3.0 | 5.0 | 3.5 |
| COLOR | 0.5 | 0.5 | 1.0 | 0.5 | 0.4 |
| HIGH INTENSITY | — | 0.2 | — | — | 0.3 |
| SWEETENER | | | | | |
| BLOAT | 1.0 | — | 0.5 | 0.2 | — |
| L. ERYTHRORHIZON | — | 1.0 | 0.5 | — | 0.2 |

TABLE 73
(DRY WEIGHT PERCENT)

| | EX. 508 | EX. 509 | EX. 510 | EX. 511 | EX. 512 |
|---|---|---|---|---|---|
| CORN SYRUP | 44.0 | — | 44.0 | 47.3 | — |
| SUGAR | 53.5 | — | 51.0 | 47.0 | — |
| POLYALCOHOL | — | 95.3 | — | — | 95.6 |
| FLAVOR | 1.0 | 3.0 | 3.0 | 5.0 | 3.5 |
| COLOR | 0.5 | 0.5 | 1.0 | 0.5 | 0.4 |
| HIGH INTENSITY | — | 0.2 | — | — | 0.3 |
| SWEETENER | | | | | |
| CHUANG LIAN | 1.0 | — | 0.5 | 0.2 | — |
| GINGER | — | 1.0 | 0.5 | — | 0.2 |

TABLE 74
(DRY WEIGHT PERCENT)

| | | | | EX. | EX. |
|---|---|---|---|---|---|
| | EX. 513 | EX. 514 | EX. 515 | 516 | 517 |
| CORN SYRUP | 44.0 | — | 44.0 | 47.3 | — |
| SUGAR | 53.5 | — | 51.0 | 47.0 | — |
| POLYALCOHOL | — | 95.3 | — | — | 95.6 |
| FLAVOR | 1.0 | 3.0 | 3.0 | 5.0 | 3.5 |
| COLOR | 0.5 | 0.5 | 1.0 | 0.5 | 0.4 |
| HIGH INTENSITY | — | 0.2 | — | — | 0.3 |
| SWEETENER | | | | | |
| HONEYSUCKLE EXTRACT | 1.0 | — | 0.5 | 0.2 | — |
| LUO HAN GUO | — | 1.0 | 0.5 | — | 0.2 |

### Pressed Mint Examples

TABLE 75
(DRY WEIGHT PERCENT)

| | Ex. 518 | Ex. 519 | Ex. 520 | Ex. 521 | Ex. 522 |
|---|---|---|---|---|---|
| SORBITOL | 97.63 | 97.43 | 96.83 | 95.83 | 94.83 |
| FLAVOR | 1.00 | 1.00 | 1.00 | 1.00 | 1.00 |
| MG STEARATE | 0.97 | 0.97 | 0.97 | 0.97 | 0.97 |
| HIGH INTENSITY | 0.20 | 0.20 | 0.20 | 0.20 | 0.20 |
| SWEETENER | | | | | |
| CHRYSANTHEMUM | 0.10 | — | 0.50 | — | — |
| FLOWER | | | | | |
| LUO HAN GUO | 0.10 | — | 0.30 | 2.00 | 1.50 |
| WOLFBERRY | — | 0.40 | 0.20 | — | 1.50 |

Healthful chewing gums of the present invention were made according to the formulas of Examples 523-528.

TABLE 76
(WEIGHT PERCENT)

| | EX. 523 | EX. 524 | EX. 525 | EX. 526 | EX. 527 | EX. 528 |
|---|---|---|---|---|---|---|
| GUM BASE | 31.50 | 31.50 | 31.50 | 31.50 | 31.50 | 31.50 |
| SORBITOL | 3.00 | 44.81 | 3.00 | 44.79 | 3.00 | 44.70 |
| XYLITOL | 60.81 | 19.00 | 60.79 | 19.00 | 60.70 | 19.00 |
| FLAVOR | 2.17 | 2.17 | 2.20 | 2.20 | 2.40 | 2.40 |
| COLOR | 0.05 | 0.05 | 0.04 | 0.04 | 0.06 | 0.06 |
| HIGH-INTENSITY | 0.30 | 0.30 | 0.30 | 0.30 | 0.67 | 0.67 |
| SWEETENER | | | | | | |
| (ENCAPSULATED) | | | | | | |
| CITRIC ACID | 1.00 | 1.00 | — | — | 0.40 | 0.40 |
| FUMARIC ACID | 0.30 | 0.30 | 0.30 | 0.30 | — | — |
| MALIC ACID | — | — | — | — | 0.40 | 0.40 |
| MCT OIL* | 0.80 | 0.80 | 0.80 | 0.80 | 0.80 | 0.80 |
| ALOE ARBORESCENS | 0.07 | 0.07 | — | — | — | — |
| WHOLE LEAF POWDER | | | | | | |
| LOQUAT FRUIT | — | — | 0.07 | 0.07 | — | — |
| (SPRAY DRIED | | | | | | |
| EXTRACT) | | | | | | |
| LUO HAN GUO | — | — | 1.00 | 1.00 | — | — |
| (SPRAY DRIED EXTRACT) | | | | | | |
| BARBARY | — | — | — | — | 0.07 | 0.07 |
| WOLFBERRY FRUIT | | | | | | |
| (SPRAY DRIED EXTRACT) | | | | | | |
| TOTAL | 100.00 | 100.00 | 100.00 | 100.00 | 100.00 | 100.00 |

*Medium Chain Triglycerides

The chewing gums of Examples 523-528 were formed into pellets and coated to a coating level of 32% according to the formula of Example 529.

TABLE 77
(WEIGHT PERCENT)

| | EX. 529 |
|---|---|
| MALTITOL | 87.62 |
| SORBITOL | 0.64 |
| VEG. GUM** | 9.01 |
| FLAVOR | 1.47 |
| POLISHING COMPOUND | 0.67 |
| COLOR | 0.59 |
| TOTAL | 100.00 |

**Gum Arabic and/or Gum Tahla

In general, aloe arborescents whole leaf powder can be used at a level of 0.03 to 0.50% by weight of the finished gum product. In general, spray dried extract of barbary wolfberry fruit can be used at a level of 0.03 to 0.50% by weight of the finished gum product. In general, spray dried extract of loquat fruit and spray dried extract of luo han guo can be used in combination at levels of 0.03 to 0.50% and 0.50 to 2.00% respectively by weight of the finished gum product.

TABLE 78
(WEIGHT PERCENT)

| | EX. 530 | EX. 531 | EX. 532 | EX. 533 | EX. 534 | EX. 535 |
|---|---|---|---|---|---|---|
| GUM BASE | 31.50 | 31.50 | 31.50 | 31.50 | 31.50 | 31.50 |
| SORBITOL | 3.00 | 3.00 | 3.00 | 3.00 | 3.00 | 3.00 |
| XYLITOL | 60.74 | 60.67 | 61.72 | 61.79 | 40.73 | 59.87 |
| FLAVOR | 2.17 | 2.17 | 2.20 | — | 2.40 | 2.40 |
| GINSENG FLAVOR | — | — | — | 2.20 | — | — |
| COLOR | 0.05 | 0.05 | 0.04 | 0.04 | 0.06 | 0.06 |
| HIGH-INTENSITY | 0.30 | 0.30 | 0.30 | 0.30 | 0.67 | 0.67 |
| SWEETENER | | | | | | |
| (ENCAPSULATED) | | | | | | |
| CITRIC ACID | 1.00 | 1.00 | — | — | 0.40 | 0.40 |
| FUMARIC ACID | 0.30 | 0.30 | 0.30 | 0.30 | — | — |
| MALIC ACID | — | — | — | — | 0.40 | 0.40 |
| MCT OIL* | 0.80 | 0.80 | 0.80 | 0.80 | 0.80 | 0.80 |
| GREEN TEA | 0.07 | — | — | — | 0.50 | — |
| EXTRACT | | | | | | |
| POMEGRANATE FRUIT | 0.07 | — | — | — | 1.00 | — |
| (SPRAY DRIED EXTRACT) | | | | | | |
| MULBERRY LEAF | — | 0.07 | — | — | — | 0.30 |
| EXTRACT | | | | | | |
| CHRYSANTHEMUM | — | 0.07 | — | — | — | 0.30 |
| EXTRACT | | | | | | |
| RADIX PLATYCODONIS | — | 0.07 | — | — | — | 0.30 |
| EXTRACT | | | | | | |
| SPINE DATE SEED | — | — | 0.07 | — | — | — |
| EXTRACT | | | | | | |
| ROSE EXTRACT | — | — | 0.07 | — | — | — |
| OOLONG TEA | — | — | — | 0.07 | — | — |
| EXTRACT | | | | | | |
| TOTAL | 100.00 | 100.00 | 100.00 | 100.00 | 100.00 | 100.00 |

*Medium Chain Triglycerides

The chewing gums of Examples 530-535 can be formed into pellets and coated to a coating level of 32% according to the formula of Example 529.

In general, green tea extract can be used at a level of 0.03 to 1.00% and pomegranate fruit spray dried extract can be used at a level of 0.03 to 3.00%. These extracts can be used separately or, more advantageously, in combination as antioxidants to reduce free radicals and enhance the appearance of skin.

In general, mulberry leaf, chrysanthemum and Radix platycodonis extracts may be used at a level of 0.03 to 0.50%. These extracts may be used separately or, more advantageously, in combination to reduce internal heat and sooth irritated throats.

In general, spine date seed extract and rose extracts may be used at a level of 0.03 to 0.50%. These extracts may be used separately or, more advantageously, in combination to reduce stress and promote relaxation.

In general, oolong tea extracts may be used at a level of 0.03 to 2.00% preferably in combination with ginseng flavor to increase alertness and mental functioning.

It should be appreciated that the compositions and methods of the present invention are capable of being incorporated in the form of a variety of embodiments, only a few of which have been illustrated and described above. The invention may be embodied in other forms without departing from its spirit or essential characteristics. The described embodiments are to be considered in all respects only as illustrative and not restrictive, and the scope of the invention, therefore, indicated by the appended claims rather than by the foregoing description. All changes which come within the meaning and range of equivalency of the claims are to be embraced within their scope.

1-68. (canceled)

69. A chewing gum product comprising a traditional Chinese medicinal herb or extract thereof selected from the group consisting of lo han guo, chrysanthemum, honeysuckle, bloat fruit, loquat, barbary wolfberry, fritillariae bulb, aloe, mulberry, Radix platycodonis, spine date seed, Chinese white olive and mixtures thereof.

70. The chewing gum product of claim 69 wherein the Chinese medicinal herb or extract thereof is present in a coating on a chewing gum center.

71. The chewing gum product of claim 69 wherein the Chinese medicinal herb or extract is encapsulated.

72. The chewing gum product of claim 69 wherein the chewing gum product further comprises a high-intensity sweetener selected from the group consisting of aspartame, acesulfame and its salts, cyclamate and its salts, saccharin and its salts, alitame, neotame, sucralose, thaumatin, monellin, dihydrochalcone, stevioside, glycyrrhizin, and combinations thereof.

73. The chewing gum product of claim 72 wherein the Chinese medicinal herb or extract thereof comprises lo han guo, and the high-intensity sweetener is selected from the group consisting of thaumatin, monellin, dihydrochalcone, stevioside, glycyrrhizin, and combinations thereof.

74. The chewing gum product of claim 73 wherein the chewing gum product further comprises a cooling agent.

75. The chewing gum product of claim 69 wherein the chewing gum product further comprises an absorption enhancing agent selected from the group consisting of menthol and menthol derivatives, limonene, carvone, isomenthol, eucalyptol, menthone, pinene, camphor and camphor derivatives, monoterpene natural products and mixtures thereof.

76. The chewing gum product of claim 69 wherein the Chinese medicinal herb or extract thereof is present at a level of 0.2% to 5% of the chewing gum product.

77. The chewing gum product of claim 69 wherein the Chinese medicinal herb or extract thereof comprises chrysanthemum, lo han guo and honeysuckle.

78. The chewing gum product of claim 77 further comprising aspartame and a salt of acesulfame.

79. The chewing gum product of claim 78 further comprising a cooling agent.

80. A method of producing a chewing gum product containing an absorption-enhanced traditional Chinese medicine active agent in order to control the absorption rate of the traditional Chinese medicine active agent comprising the steps of:
a) mixing a quantity of a traditional Chinese medicinal herb or extract thereof selected from the group consisting of lo han guo, chrysanthemum, honeysuckle, bloat fruit, loquat, barbary wolfberry, fritillariae bulb, aloe, mulberry, Radix platycodonis, spine date seed, Chinese white olive and mixtures thereof with an absorption enhancing agent selected from the group consisting of menthol and menthol derivatives, limonene, carvone, isomenthol, eucalyptol, menthone, pinene, camphor and camphor derivatives, monoterpene natural products; and
b) adding a quantity of the mixture to a chewing gum formulation to provide a traditional Chinese medicine active agent level in the chewing gum formulation of from 12 micrograms to 250 milligrams per gram of chewing gum product.

81. The method of claim 80 wherein the chewing gum formulation comprises a high-intensity sweetener selected from the group consisting of aspartame, acesulfame and its salts, cyclamate and its salts, saccharin and its salts, alitame, neotame, sucralose, thaumatin, monellin, dihydrochalcone, stevioside, glycyrrhizin, and combinations thereof.
